(19) United States
(12) Patent Application Publication
(10) Pub. No.: US 2024/0238413 A1
(43) Pub. Date: Jul. 18, 2024

(54) HEPATITIS C VIRUS IMMUNOGENIC COMPOSITIONS AND METHODS OF USE THEREOF

(71) Applicant: The Governors of the University of Alberta, Edmonton (CA)

(72) Inventors: Michael Houghton, Alberta (CA); Abdolamir Landi, Alberta (CA); John L. Law, Alberta (CA)

(21) Appl. No.: 18/391,501

(22) Filed: Dec. 20, 2023

Related U.S. Application Data
(63) Continuation of application No. PCT/CA2023/050407, filed on Mar. 27, 2023.
(60) Provisional application No. 63/397,676, filed on Aug. 12, 2022.
(60) Provisional application No. 63/358,332, filed on Jul. 5, 2022.
(60) Provisional application No. 63/324,339, filed on Mar. 28, 2022.

(51) Int. Cl. A61K 39/29; A61K 39/00; A61K 39/39; A61P 37/04

(52) U.S. Cl. CPC A61K 39/29; A61K 39/39; A61P 37/04; A61K 2039/55505; A61K 2039/55511; A61K 2039/55516; A61K 2039/55555; A61K 2039/55561; A61K 2039/55572; A61K 2039/55577

(57) ABSTRACT

The present disclosure provides immunogenic compositions comprising one or more T-cell epitope polypeptides, or a fusion polypeptide comprising two or more T-cell epitope polypeptides. The present disclosure provides a method for inducing an immune response to hepatitis C virus in an individual, the method comprising administering to the individual an immunogenic composition of the present disclosure.

### CROSS-REFERENCE

This application claims the benefit of U.S. Provisional Patent Application No. 63/324,339, filed Mar. 28, 2022, U.S. Provisional Patent Application No. 63/358,332, filed Jul. 5, 2022, and U.S. Provisional Patent Application No. 63/397,676, filed Aug. 12, 2022, which applications are incorporated herein by reference in their entirety.

### INCORPORATION-BY-REFERENCE OF MATERIAL ELECTRONICALLY SUBMITTED

A Sequence Listing is provided herewith as a Sequence Listing XML, “UALB-058WO_SEQ_LIST” created on Mar. 26, 2023 and having a size of 255,129 bytes. The contents of the Sequence Listing XML are incorporated by reference herein in their entirety.

### INTRODUCTION

Hepatitis C virus (HCV) is a blood-borne pathogen that is estimated to infect 150-200 million people worldwide. Infection by HCV may be non-symptomatic, and can be cleared by patients, sometimes without medical intervention. However, the majority of patients develop a chronic HCV infection, which may lead to liver inflammation, scarring, and even to liver failure or liver cancer. In the United States alone, over 3 million people have a chronic infection.

The HCV virion contains a positive-sense single stranded RNA genome of about 9.5 kb. The genome encodes a single polyprotein of 3,010 to 3,030 amino acids. The structural proteins comprise a core protein forming the viral nucleocapsid and two envelope glycoproteins, E1 and E2.

There is a need in the art for compositions and methods for inducing immune responses to HCV.

### SUMMARY

The present disclosure provides immunogenic compositions comprising one or more T-cell epitope polypeptides, or a fusion polypeptide comprising two or more T-cell epitope polypeptides. The present disclosure provides a method for inducing an immune response to hepatitis C virus in an individual, the method comprising administering to the individual an immunogenic composition of the present disclosure.

### BRIEF DESCRIPTION OF THE DRAWINGS

FIG. 1A-1J provide an amino acid sequence alignment of examples of the core-E1-E2 coding regions of an HCV genotype 1 virus, specifically representative HCV 1A, 1B and 1C genotypes. Genbank database sequences for the coding region core-E1-E2 were aligned using Geneious software v5.6.4. Numbering of amino acids is according to strain NP_671941 (H77). Consensus: SEQ ID NO:1; AVI1a129: SEQ ID NO:2; NP_671491 (H77): SEQ ID NO:3; EU155269: SEQ ID NO:4; EU781810: SEQ ID NO:5; EU781771: SEQ ID NO:6; AB250610: SEQ ID NO:7; EU781752: SEQ ID NO:8; EU781759: SEQ ID NO:9; EF407439: SEQ ID NO:10; EF407427: SEQ ID NO:11; EU362905: SEQ ID NO:11; EF407413: SEQ ID NO:11; EU781808: SEQ ID NO:14; EU78170: SEQ ID NO:15; AJ238799 (Con1): SEQ ID NO:16; AAK97744: SEQ ID NO:17; AF139594: SEQ ID NO:18; AF176573: SEQ ID NO:19; BAA19625: SEQ ID NO:20; BAA25076: SEQ ID NO:21; BAC54896: SEQ ID NO:22; BAD91386: SEQ ID NO:23; BAF46764: SEQ ID NO:24; BAG30950: SEQ ID NO:24; CAB41951: SEQ ID NO:26; AAK95832: SEQ ID NO:27; AAT69968: SEQ ID NO:28; and BAA03581: SEQ ID NO:29.

FIG. 2A-2F provide an alignment of amino acid sequences of the core-E1-E2 coding region of representative HCV 2A and HCV2B subtypes. Genbank database sequences for the coding region core-E1-E2 were aligned using Geneious software v5.6.4. The amino acid numbering depicted is in accordance with the common HCV strains: D00994 (HC-J6) for HCV 2A; AB047639 (JFH1) and HPCJ8G-J8 (J8) for HCV2A and HCV2B, respectively. AB047639 (JFH1): SEQ ID NO:30; AB047645: SEQ ID NO:31; AF169003: SEQ ID NO:32; AF169005: SEQ ID NO:33; AF238482: SEQ ID NO:34; AY746460: SEQ ID NO:35; HPCPOLP: SEQ ID NO:36; NC_009823: SEQ ID NO:37; HPCJ8G HC-J8: SEQ ID NO:38; AB030907: SEQ ID NO:39; AY232730: SEQ ID NO:40; AY232747: SEQ ID NO:41; and DQ430817: SEQ ID NO:42.

FIG. 3A-3F provide an amino acid sequence alignment of the core-E1-E2 coding region for representative HCV 3A, 3B and 3K genotypes. Genbank database sequences for the coding region core-E1-E2 were aligned using Geneious software v5.6.4. Consensus: SEQ ID NO:43; AVI3a177: SEQ ID NO:44; ADF97232(S52): SEQ ID NO:45; YP_0014696: SEQ ID NO:46; CAA54244: SEQ ID NO:47; AAC03058: SEQ ID NO:48; AAY29642: SEQ ID NO:49; ABD85062: SEQ ID NO:50; ABD85063: SEQ ID NO:50; ABD97104: SEQ ID NO:52; BAA06044: SEQ ID NO:53; BAA08372: SEQ ID NO:54; and BAA09890: SEQ ID NO:55.

FIG. 4A-4B provide an amino acid sequence of the core-E1-E2 coding region for HCV genotype 7a. Amino acid sequence for the coding region core-E1-E2 of genotype 7a (isolate QC69; Genbank: ABN05226.1; SEQ ID NO:56) is shown according to the numbering scheme of the reference strain, NP_671941 (H77).

FIG. 5 depicts HCV E1/E2-specific IgG levels in post-vaccinated mouse sera.

FIG. 6 depicts neutralization of HCV pseudoparticles with post-vaccinated mouse sera.

FIG. 7A-7B depict data showing that E1E2-4TPs mixtures adjuvanted with SLA-LSQ or SLA-SE can induce generation of T cells that produce IFN-γ, IL-2, and TNF-α.

FIG. 8A-8B depict structures of several saponins.

FIG. 9 depicts the structures of squalene, miglyol 810, DOPC, Tween80 and Pluronic F68.

FIG. 10 provides the amino acid sequence of a TP fusion polypeptide (SEQ ID NO:57).

FIG. 11A-11C provide amino acid sequences of TP465 polypeptides (SEQ ID Nos:58-60, respectively).

FIG. 12A-12E provides amino acid sequences of examples of T-cell epitope polypeptides (“TPs”) (SEQ ID Nos:61-70, respectively).

FIG. 13A-13B depict data showing that E1E2-4TPs mixtures adjuvanted with SLA-LSQ or SLA-SE can, when stimulated in vitro with individual TPs, induce generation of CD4⁺ T cells that produce IFN-γ, IL-2, and TNF-α.

FIG. 14A-14B depict data showing that E1E2-4TPs mixtures adjuvanted with SLA-LSQ or SLA-SE can, when stimulated in vitro with individual TPs, induce generation of CD8⁺ T cells that produce IFN-γ, IL-2, and TNF-α.

### DEFINITIONS

The term “hepatitis C virus” (“HCV”), as used herein, refers to any one of a number of different genotypes and isolates of hepatitis C virus. Thus, “HCV” encompasses any of a number of genotypes, subtypes, or quasispecies, of HCV, including, e.g., genotype 1, 2, 3, 4, 5, 6, 7, etc. and subtypes (e.g., 1a, 1b, 2a, 2b, 3a, 4a, 4c, etc.), and quasispecies. Representative HCV genotypes and isolates include: HCV-1, H77, J6, Con1, isolate 1, BK, EC1, EC10, HC-J2, HC-J5; HC-J6, HC-J7, HC-J8, HC-JT, HCT18, HCT27, HCV-476, HCV-KF, “Hunan”, “Japanese”, “Taiwan”, TH, type 1, type 1a, H77 type 1b, type 1c, type 1d, type 1e, type 1f, type 10, type 2, type 2a, type 2b, type 2c, type 2d, type 2f, type 3, type 3a, type 3b, type 3g, type 4, type 4a, type 4c, type 4d, type 4f, type 4h, type 4k, type 5, type 5a, type 6, type 6a, and type 7a.

The terms “individual,” “host,” “subject,” and “patient” are used interchangeably herein, and refer to a mammal, including, but not limited to, non-human primates (e.g., simians), equines (e.g., horses), rodents (e.g., rats; mice), and humans.

As used herein, the term “isolated,” in reference to a polypeptide, refers to a polypeptide that is in an environment different from that in which the polypeptide naturally occurs. An isolated polypeptide can be purified. By “purified” is meant a compound of interest (e.g., a polypeptide) has been separated from components that accompany it in nature. “Purified” can also be used to refer to a polypeptide separated from components that can accompany it during production of the polypeptide (e.g., during synthesis in vitro, etc.). In some embodiments, a polypeptide (or a mixture of polypeptides) is substantially pure when the polypeptide (or mixture of polypeptides) is at least 60% or at least 75% by weight free from organic molecules with which it is naturally associated or with which it is associated during production. In some cases, the polypeptide is from 30% to 60% pure. In some cases, the polypeptide (or mixture of polypeptides) is at least 60%, at least 75%, at least 80%, at least 85%, at least 90%, at least 95%, or at least 99%, by weight, pure. For example, in some cases, an E1 or an E2 polypeptide (or a mixture of E1 and E2 polypeptides, e.g., an E1/E2 heterodimer) is substantially pure when the E1 or E2 polypeptide (or mixture of E1 and E2 polypeptides) is at least 60% or at least 75% by weight free from organic molecules with which the polypeptide(s) is naturally associated or with which it is associated during production. In some cases, the E1 or E2 polypeptide (or mixture of E1 and E2 polypeptides) is at least 60%, at least 75%, at least 80%, at least 85%, at least 90%, at least 95%, or at least 99%, by weight, pure. In some cases, where a composition comprises an E2 polypeptide, the E2 polypeptide is at least 60%, at least 75%, at least 80%, at least 85%, at least 90%, at least 95%, or at least 99%, by weight, pure. In some cases, where a composition comprises an E1/E2 heterodimeric complex polypeptide, the E1/E2 heterodimeric complex polypeptide is at least 60%, at least 75%, at least 80%, at least 85%, at least 90%, at least 95%, or at least 99%, by weight, pure. In some cases, where a composition comprises a T-cell epitope polypeptide, the T-cell epitope polypeptide is at least 60%, at least 75%, at least 80%, at least 85%, at least 90%, at least 95%, or at least 99%, by weight, pure.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term “polypeptide” includes glycosylated polypeptides.

The term “heterologous” refers to two components that are defined by structures derived from different sources. For example, where “heterologous” is used in the context of a polypeptide, the polypeptide includes operably linked amino acid sequences that can be derived from one or more different polypeptides, e.g., amino acid sequences that are not operably linked to the polypeptide in nature. As another example, where a composition comprises an HCV E1/E2 heterodimer and a “heterologous” polypeptide, the “heterologous polypeptide is a polypeptide other than HCV E1 or HCV E2. As another example, where a fusion polypeptide comprises: a) a T-cell epitope polypeptide (a “TP”); and b) a heterologous fusion partner polypeptide, the “heterologous fusion partner polypeptide” is one that is not found associated with the T-cell epitope polypeptide in nature. As another example, where “heterologous” is used in the context of a nucleic acid or nucleotide sequence, the nucleic acid includes operably linked nucleotide sequences that are not normally linked in nature. For example, an IRES can be heterologous to a nucleotide sequence encoding an HCV E1 and/or E2 polypeptide, where the IRES is from an organism (e.g., a virus) other than HCV.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well known to those of skill in the art. See, e.g., Pearson (1994) Methods Mol. Biol. 24: 307-331, herein incorporated by reference. Examples of groups of amino acids that have side chains with similar chemical properties, and that thus constitute conservative amino acid substitution groups, include: 1) aliphatic side chain-containing amino acids: glycine, alanine, valine, leucine and isoleucine; 2) aliphatic-hydroxyl side chain-containing amino acids: serine and threonine; 3) amino acids with amide-containing side chains: asparagine and glutamine; 4) aromatic side chain-containing amino acids: phenylalanine, tyrosine, and tryptophan; 5) amino acids with basic side chains: lysine, arginine, and histidine; 6) amino acids with acidic side chains: aspartate and glutamate, and 7) amino acids with sulfur-containing side chains: cysteine and methionine. Examples of conservative amino acid substitution groups are: valine-leucine-isoleucine, phenylalanine-tyrosine, lysine-arginine, alanine-valine, glutamate-aspartate, and asparagine-glutamine.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an HCV E1 polypeptide” includes a plurality of such polypeptides and reference to “the QS21 molecule” includes reference to one or more QS21 molecules and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides immunogenic compositions comprising: a) one or more T-cell epitope polypeptides, or a fusion polypeptide comprising two or more T-cell epitope polypeptides; and b) a second-generation lipid adjuvant (SLA) that is a toll-like receptor 4 (TLR4) agonist formulated in: i) a stable emulsion; or ii) a liposomal composition comprising a saponin. The present disclosure provides immunogenic compositions comprising: a) one or more T-cell epitope polypeptides, or a fusion polypeptide comprising two or more T-cell epitope polypeptides; b) a hepatitis C virus (HCV) E1 and/or an HCV E2 polypeptide; and c) SLA formulated in: i) a stable emulsion; or ii) a liposomal composition comprising a saponin. The present disclosure provides methods of inducing an immune response to HCV in an individual, the methods comprising administering to the individual an effective amount of an immunogenic composition of the present disclosure.

As noted above, in some cases, an immunogenic composition of the present disclosure comprises both one or more T-cell epitope polypeptides, or a fusion polypeptide comprising two or more T-cell epitope polypeptides; and HCV E1 and/or E2 polypeptides. The HCV E1 and/or E2 polypeptides function as the humoral element (i.e., to induce production of neutralizing antibodies) and the T-cell epitope polypeptide(s) (or fusion polypeptide comprising T-cell epitope polypeptides) function to induce CD4+ and CD8⁺ T cell responses. It was found that including in the immunogenic composition an adjuvant comprising SLA, either in a liposomal composition comprising a saponin or as a stable emulsion, induced robust CD4+ and CD8⁺ T cell responses. In particular, use of adjuvant comprising SLA, either in a liposomal composition comprising a saponin or as a stable emulsion, induced much more robust CD4+ and CD8⁺ T cell responses than when alum-OH/MPLA or AddaAS03 was used as an adjuvant.

### Immunogenic Compositions

The present disclosure provides immunogenic compositions comprising: a) one or more T-cell epitope polypeptides, or a fusion polypeptide comprising two or more T-cell epitope polypeptides; and b) SLA formulated in: i) a stable emulsion; or ii) a liposomal composition comprising a saponin. The present disclosure provides immunogenic compositions comprising: a) one or more T-cell epitope polypeptides, or a fusion polypeptide comprising two or more T-cell epitope polypeptides; b) an HCV E1 and/or an HCV E2 polypeptide; and c) SLA formulated in: i) a stable emulsion; or ii) a liposomal composition comprising a saponin.

An immunogenic composition of the present disclosure can, when administered to an individual, induce a more robust CD4⁺ T cell response and/or a more robust CD8⁺ T cell response than a reference immunogenic composition comprising Alum-OH/MPLA or AS03 as the adjuvant, where the reference immunogenic composition comprises the same T-cell epitope polypeptides, or fusion polypeptide comprising T-cell epitope polypeptides, as an immunogenic composition of the present disclosure, or where the reference immunogenic composition comprises the same T-cell epitope polypeptides, or fusion polypeptide comprising T-cell epitope polypeptides, and the same HCV E1 and/or E2 polypeptides, as an immunogenic composition of the present disclosure. In other words, for comparison, the reference immunogenic composition differs from an immunogenic composition of the present disclosure only in the adjuvant that is included.

In some cases, an immunogenic composition of the present disclosure induces a CD4⁺ T cell response to HCV that is at least 10%, at least 15%, at least 25%, at least 50%, at least 75%, at least 100% (or two-fold), at least 2.5-fold, at least 5-fold, at least 10-fold, at least 25-fold, or more than 25-fold, greater than the CD4⁺ T cell response induced with a reference immunogenic composition. In some cases, an immunogenic composition of the present disclosure induces a CD8⁺ T cell response to HCV that is at least 10%, at least 15%, at least 25%, at least 50%, at least 75%, at least 100% (or two-fold), at least 2.5-fold, at least 5-fold, at least 10-fold, at least 25-fold, or more than 25-fold, greater than the CD8⁺ T cell response induced with a reference immunogenic composition. In some cases, an immunogenic composition of the present disclosure induces a CD4⁺ T cell response and a CD8⁺ T cell response to HCV that are at least 10%, at least 15%, at least 25%, at least 50%, at least 75%, at least 100% (or two-fold), at least 2.5-fold, at least 5-fold, at least 10-fold, at least 25-fold, or more than 25-fold, greater than the CD4⁺ T cell response and CD8⁺ T cell response induced with a reference immunogenic composition.

For example, an immunogenic composition of the present disclosure comprising: a) one or more T-cell epitope polypeptides, or a fusion polypeptide comprising two or more T-cell epitope polypeptides; b) an HCV E1 and/or an HCV E2 polypeptide; and c) SLA formulated in: i) a stable emulsion; or ii) a liposomal composition comprising a saponin can induce a CD4⁺ T cell response to HCV that is at least 10%, at least 15%, at least 25%, at least 50%, at least 75%, at least 100% (or two-fold), at least 2.5-fold, at least 5-fold, at least 10-fold, at least 25-fold, or more than 25-fold, greater than the CD4⁺ T cell response induced with a reference immunogenic composition comprising: a) one or more T-cell epitope polypeptides, or a fusion polypeptide comprising two or more T-cell epitope polypeptides; b) an HCV E1 and/or an HCV E2 polypeptide; and c) Alum-OH/MPLA.

As another example, an immunogenic composition of the present disclosure comprising: a) one or more T-cell epitope polypeptides, or a fusion polypeptide comprising two or more T-cell epitope polypeptides; b) an HCV E1 and/or an HCV E2 polypeptide; and c) SLA formulated in: i) a stable emulsion; or ii) a liposomal composition comprising a saponin can induce a CD8⁺ T cell response to HCV that is at least 10%, at least 15%, at least 25%, at least 50%, at least 75%, at least 100% (or two-fold), at least 2.5-fold, at least 5-fold, at least 10-fold, at least 25-fold, or more than 25-fold, greater than the CD8⁺ T cell response induced with a reference immunogenic composition comprising: a) one or more T-cell epitope polypeptides, or a fusion polypeptide comprising two or more T-cell epitope polypeptides; b) an HCV E1 and/or an HCV E2 polypeptide; and c) Alum-OH/MPLA.

As another example, an immunogenic composition of the present disclosure comprising: a) one or more T-cell epitope polypeptides, or a fusion polypeptide comprising two or more T-cell epitope polypeptides; b) an HCV E1 and/or an HCV E2 polypeptide; and c) SLA formulated in: i) a stable emulsion; or ii) a liposomal composition comprising a saponin can induce a CD4⁺ T cell response to HCV that is at least 10%, at least 15%, at least 25%, at least 50%, at least 75%, at least 100% (or two-fold), at least 2.5-fold, at least 5-fold, at least 10-fold, at least 25-fold, or more than 25-fold, greater than the CD4⁺ T cell response induced with a reference immunogenic composition comprising: a) one or more T-cell epitope polypeptides, or a fusion polypeptide comprising two or more T-cell epitope polypeptides; b) an HCV E1 and/or an HCV E2 polypeptide; and c) AS03.

As another example, an immunogenic composition of the present disclosure comprising: a) one or more T-cell epitope polypeptides, or a fusion polypeptide comprising two or more T-cell epitope polypeptides; b) an HCV E1 and/or an HCV E2 polypeptide; and c) SLA formulated in: i) a stable emulsion; or ii) a liposomal composition comprising a saponin can induce a CD8⁺ T cell response to HCV that is at least 10%, at least 15%, at least 25%, at least 50%, at least 75%, at least 100% (or two-fold), at least 2.5-fold, at least 5-fold, at least 10-fold, at least 25-fold, or more than 25-fold, greater than the CD8⁺ T cell response induced with a reference immunogenic composition comprising: a) one or more T-cell epitope polypeptides, or a fusion polypeptide comprising two or more T-cell epitope polypeptides; b) an HCV E1 and/or an HCV E2 polypeptide; and c) AS03.

Alum-OH/MPLA is an adjuvant comprising: a) aluminum hydroxide; and b) monophosphoryl lipid A (MPLA). AddaS03™ is an oil-in-water nano-emulsion adjuvant comprising squalene, DL-α-tocopherol, and polysorbate 80 (Tween® 80). AS03 is an oil-in-water emulsion comprising squalene, polysorbate 80 and α-tocopherol. A dose of AS03 can contain: a) 10.69 mg squalene; b) 11.86 mg DL-α-tocopherol; and c) 4.86 mg polysorbate 80.

Squalene has the following structure:

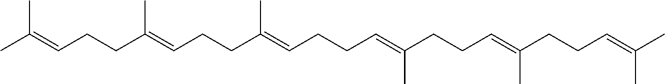 [SMILES: CC(C)=CCC/C(C)=C/CC/C(C)=C/CC/C=C(\C)CC/C=C(\C)CCC=C(C)C]

Polysorbate 80 has the following structure:

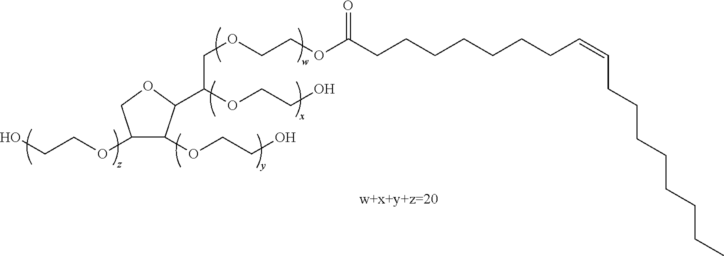

### Liposomal Compositions Comprising a Saponin (SLA-Liposomal Compositions)

As noted above, in some cases, an immunogenic composition of the present disclosure comprises SLA in a liposomal composition comprising a saponin. See, e.g., US 2020/0276299. SLA in a liposomal composition comprising a saponin is also referred to herein as “SLA-LSQ”.

In some cases, the saponin is present in the liposomal composition at a concentration of from about 1 μg per unit dose to about 10 μg per unit dose, or from about 1 μg per dose to about 8 μg per unit dose. In some cases, a unit dose is 0.5 mL.

In some cases, the weight of ratio of SLA to saponin is about 2.5 to 1.

In some cases, a liposomal composition comprising a saponin is complexed to a sterol. In some cases, the weight:weight ratio of saponin to sterol is from about 1:110 to about 1:200, from about 1:110 to 1:150, from about 1:120 to about 1:150, or about 1:125. In some cases, the weight:weight ratio of saponin to sterol is 1:125. In some cases, the sterol is cholesterol. In some cases, the saponin is QS21 and the sterol is cholesterol.

The saponin can be present in the formulation in a concentration of from about 2 μg per mL to about 8 μg per mL. In some cases, a unit dose of an SLA-liposomal composition comprises from 2 μg to 4 μg saponin. In some cases, a unit dose is 0.5 mL.

The SLA can be present in the formulation in a concentration of from about 5 μg per unit dose to about 10 μg per unit dose. In some cases, a unit dose is 0.5 mL.

In some cases, an SLA-liposomal composition comprises: i) 20 μg/mL SLA; ii) 8 μg/mL QS-21; iii) cholesterol; and iv) dioleoyl phosphatidylcholine; where the QS21 and the cholesterol are present in a weight:weight (w:w) ratio of 1:125. The dioleoyl phosphatidylcholine and the cholesterol can be present in a 4:1 w:w ratio.

The liposomes can have an average diameter (i.e., the number average diameter) of 1 micrometer or less. In some cases, the average particle size (i.e., the number average diameter) of the liposome particles is about 900 nm or less, about 800 nm or less, about 700 nm or less, about 600 nm or less, about 500 nm or less, about 400 nm or less, 300 nm or less, or 200 nm or less, for example, from about 50 nm to about 900 nm, from about 50 nm to about 800 nm, from about 50 nm to about 700 nm, from about 50 nm to about 600 nm, from about 50 nm to about 500 nm, from about 50 nm to about 400 nm, from about 50 nm to about 300 nm, from about 50 nm to about 200 nm, from about 50 nm to about 175 nm, from about 50 nm to about 150 nm, from about 50 nm to about 125 nm, from about 50 nm to about 100 nm. The size of the liposomes is about 80 nm, is about 85 nm, is about 90 nm, is about 95 nm, is about 100 nm, is about 105 nm, is about 110 nm, is about 115 nm, is about 120 nm, is about 125 nm, is about 130 nm, is about 135 nm, is about 140 nm, is about 145 nm, is about 150 nm, is about 155 nm, is about 160 nm, is about 165 nm, is about 170 nm, is about 175 nm, is about 180 nm, is about 185 nm, is about 190 nm, is about 195 nm, or is about 200 nm.

### Phospholipids

The SLA-liposomal composition can comprise a phospholipid. Suitable phospholipids include 1,2-dilauroyl-sn-glycero-3-phosphocholine (DLPC); 1,2-Dimyristoyl-sn-Glycero-3-phosphatidylcholine (DMPC); 1,2-Dipalmitoyl-sn-Glycero-3-phosphatidylcholine (DPPC); 1,2-Distearoyl-sn-Glycero-3-phosphatidylcholine (DSPC); 1,2-Dioleoyl-sn-Glycero-3-phosphatidylcholine (DOPC); 1-Palmitoyl,2-oleoyl-sn-Glycero-3-phosphatidylcholine (POPC); distearoyltrimethylammonium propane (DSTAP); dipalmitoyl(C16:0)trimethyl ammonium propane (DPTAP); 1,2-Diostearoyl-sn-Glycero-3-phosphatidylethanolamine (DSPE); 1,2-Dipalmitoyl-sn-Glycero-3-phosphatidylethanolamine (DPPE); 1,2-Dimyristoyl-sn-Glycero-3-phosphatidylethanolamine (DMPE); 1,2-Dilauroyl-sn-glycero-3-phosphorylglycerol) (DLPG); 1,2-Dimyristoyl-sn-glycero-3-phosphoglycerol (DMPG); 1,2-Dipalmitoyl-sn-glycero-3-phosphoglycerol (DPPG); 1,2-Distearoyl-sn-glycero-3-phosphoglycerol (DSPG); 1,2-Dioleoyl-sn-glycero-3-phosphoglycerol (DOPG); 1,2-dilauroyl-sn-Glycero-3-phosphatidylethanolamine (DLPE); 1,2-Dilauroyl-sn-Glycero-3-phosphatidylserine (DLPS); 1,2-dilauroyl-sn-glycero-3-phospho-L-serine; DMPS: 1,2-myristoyl-sn-glycero-3-phospho-L-serine; DPPS:1,2-dipalmitoyl-sn-glycero-3-phospho-L-serine, DSPS 1,2-distearoyl-sn-glycero-3-phospho-L-serine; DOPS: 1,2-dioleoyl-sn-glycero-3-phospho-L-serine; POPS: 1-palmitoyl-2-oleoyl-sn-glycero-3-phospho-L-serine; DLPI: 1,2-dilauroyl-sn-glycero-3-phospho-(1′-myo-inositol); DMPI: 1,2-myri stoyl-sn-glycero-3-phospho-(1′-myo-inositol); DPPI: 1,2-dipalmitoyl-sn-glycero-3-phospho-(1′-myo-inositol); DSPI: 1,2-distearoyl-sn-glycero-3-phosphoinositol; DOPI: 1,2-dioleoyl-sn-glycero-3-phospho-(1′-myo-inositol); and POPI: 1-palmitoyl-2-oleoyl-sn-glycero-3-phosphoinositol. In some cases, the phospholipid is selected from DLPC, DMPC, DPPC, DSPC, DOPC, POPC, DLPG, DMPG, DPPG, DSPG, DOPG, DSTAP, DPTAP, DSPE, DPPE, DMPE, and DLPE.

### Saponins

In some cases, the saponin is an immunologically active saponin fraction derived from the bark of Quillaja saponaria Molina. In some cases, the saponin is QS21.

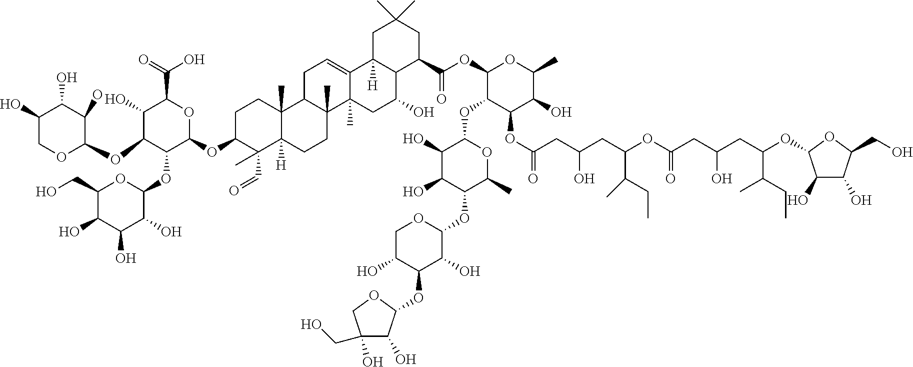

QS21 has the following structure:

In some cases, the saponin is a synthetic saponin, such as synthetic QS21 (SQS21), QS21-Api, or QS21-Xyl. See, e.g., Ragupathi et al. (2011) Expert Rev. Vaccines 10:463.

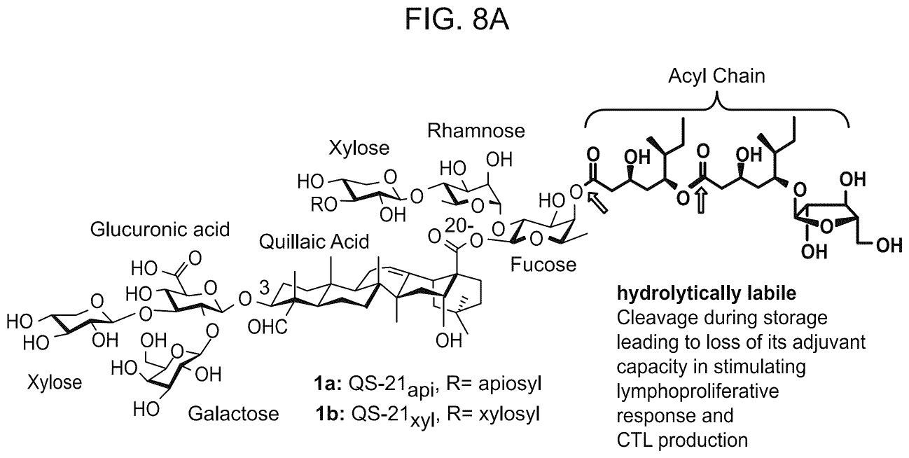

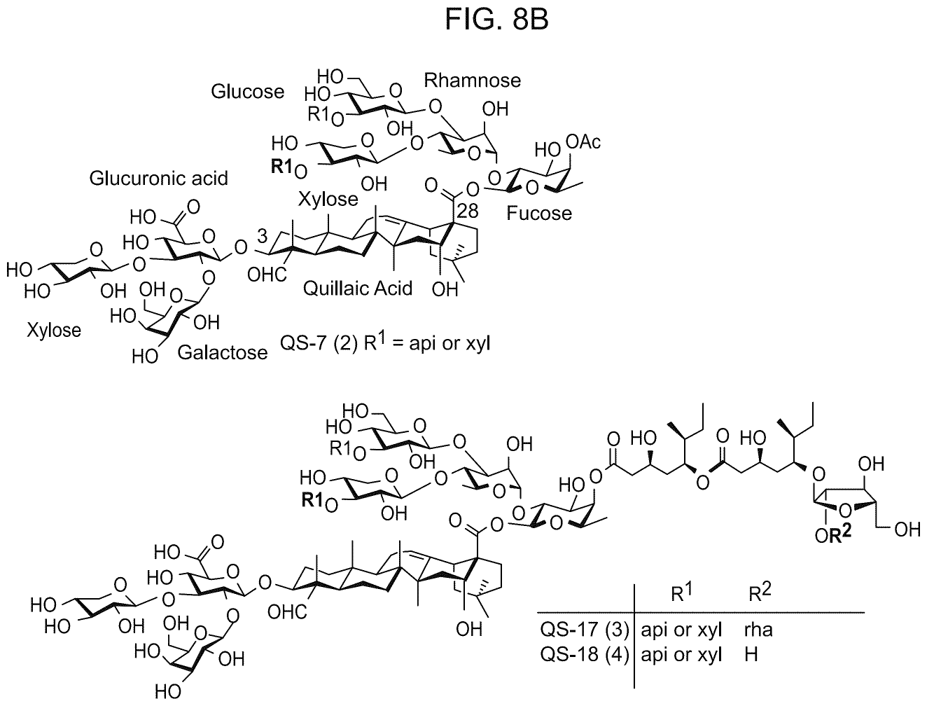

As shown in FIG. 8A, naturally occurring QS-21 has: (i) a tripterpene aglycone core, quillaic acid; (ii) a branched trisaccharide connecting to the C3 OH group of the quillaic acid through a beta glycosidic ether bond; and (iii) a linear tetrasaccharide connecting to the C28 carboxyl group of the quillaic acid through a beta glycosidic ester bond. The reducing end of the C28 tetrasaccharide is a 3-D-fucosyl unit with its 4-0 position capped with a glycosylated pseudodimeric fatty acyl chain. QS-21 is a mixture of two isomeric bidesmosidic saponins. The two structural isomers of QS-21 differ in the non-reducing end of the C28 tetrasaccharide. One isomer, QS-21ₐₚᵢ, has a terminal β-D-apiosyl unit; the other isomer, QS-21 X_{y}, has a terminal β-D-xylosyl unit.

In some cases, the saponin comprises Quil-A, or a derivative thereof, such as QS7. Other suitable saponins include Escin, Digitonin, a Gypsophila saponin, or a Chenopodium quinoa saponin. In some cases, the saponin comprises QS-7. In some cases, the saponin comprises QS-17. In some cases, the saponin comprises QS-18. The structures of QS-7, QS-17, and QS-18 are depicted in FIG. 8B.

### Stable Emulsions

As noted above, in some cases, an immunogenic composition of the present disclosure comprises SLA in a stable oil-in-water emulsion (“SE”). SLA in a stable oil-in-water emulsion is also referred to herein as “SLA-SE”. US 2015/0017191.

In some cases, an SLA-SE is an emulsion comprising: a) an aqueous phase comprising: i) an ammonium phosphate buffer; ii) a detergent (e.g., Pluronic F68 or Tween 80); and iii) glycerol; and b) an oil phase comprising: i) SLA; ii) phosphatidylcholine (PC); and iii) squalene. Miglyol 810 can be used instead of squalene. In some cases, the SLA-SE comprises, as the PC, 1,2-dimyristoyl-sn-glycero-3-phosphocholine (DMPC). In some cases, the SLA-SE does not comprise an antioxidant. In some cases, the SLA-SE does comprise an antioxidant, e.g., where the antioxidant is a tocopherol (vitamin E). In some cases, the detergent is Pluronic F68.

In some cases, the squalene is present in a concentration of from about 0.01% v/v to about 1% v/v. The hydrophobic:lipophilic balance of the emulsion is generally greater than about 9 (e.g., greater than 10, or between 9-12). In some cases, the squalene is present in a concentration of from about 0.01% v/v to 0.5% v/v. In some cases, the emulsion comprises, as the PC, 1,2-dimyristoyl-sn-glycero-3-phosphocholine (DMPC). In some cases, the emulsion comprises, as the PC, DOPC.

The PC can be egg yolk PC, soy PC, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine, 1,2-dilinoleoyl-sn-glycero-3-phosphocholine, or 1,2-diarachidonoyl-sn-glycero-3-phosphocholine. In some cases, the PC is DOPC.

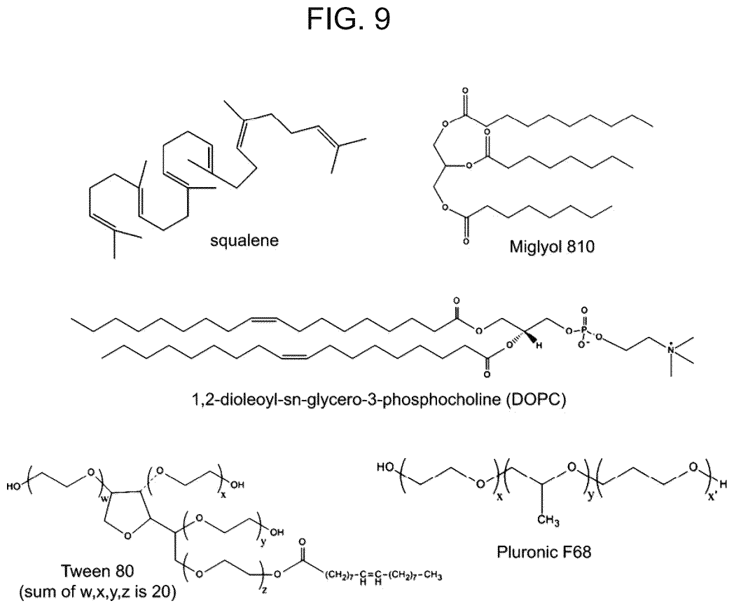

The structures of squalene, miglyol 810, DOPC, Tween 80, and Pluronic F68 are depicted in FIG. 9.

In some cases, an SLA-SE comprises: i) 10% weight/volume squalene; ii) 0.25 mg/mL SLA; iii) 0.5% weight/volume vitamin E; iv) glycerol; and v) PC.

In some cases, the SLA-SE comprises components as set out in the Table below.

| Component | Weight or volume % | | Concentration (mg/ml) |
|---|---|---|---|
| Squalene | 4.00% | v/v | 34.34 |
| Glycerol | 0.720% | v/v | 9.09 |
| PC | 0.76% | w/v | 7.84 |
| SLA | 0.04% | w/v | 0.4 |
| Pluronic F68 or Tween 80 | 0.036% or 0.21% s/v | | 0.36 or 2.13 |
| 25 mM ammonium phosphate | 95.28% | v/v | |

buffer

### TLR7/8 Agonists

The present disclosure provides immunogenic compositions comprising: a) a toll-like receptor (TLR) 7/8 agonist; and b) one or more T-cell epitope polypeptides (one or more “TPs”). The present disclosure provides immunogenic compositions comprising: a) TLR 7/8 agonist; and b) a fusion polypeptide comprising two or more TPs. The present disclosure provides immunogenic compositions comprising: a) TLR 7/8 agonist; b) one or more TPs; and c) an HCV E1 polypeptide, an HCV E2 polypeptide, or an HCV E1/E2 heterodimer. The present disclosure provides immunogenic compositions comprising: a) TLR 7/8 agonist; b) a fusion polypeptide comprising two or more TPs; and c) an HCV E1 polypeptide, an HCV E2 polypeptide, or an HCV E1/E2 heterodimer.

In some cases, the TLR 7/8 agonist is 3M-052 (also referred to as “Telratolimod” or “MEDI9197”). The 3M-052 adjuvant is an imidazoquinoline that has an 18-C fatty acyl chain which confers enhanced hydrophobicity, reduced systemic dissemination, and improved bioavailability. In some cases, 3M-052 is formulated with poly(lactic-co-glycolic) (PLGA). In some cases, 3M-052 is formulated with PLGA nanoparticles. In some cases, 3M-052 is formulated with alum.

3M-052 has the following structure:

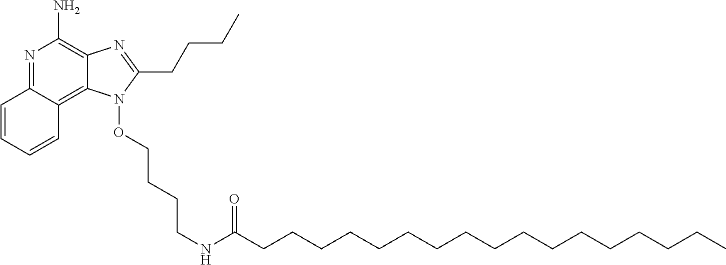 [SMILES: CCCCCCCCCCCCCCCCCC(=O)NCCCCOn1c(CCCC)nc2c(N)nc3ccccc3c21]

### Additional Adjuvants

The present disclosure provides immunogenic compositions comprising: a) one or more adjuvants; and b) one or more T-cell epitope polypeptides (one or more “TPs”). The present disclosure provides immunogenic compositions comprising: a) one or more adjuvants; and b) a fusion polypeptide comprising two or more TPs. The present disclosure provides immunogenic compositions comprising: a) one or more adjuvants; b) one or more TPs; and c) an HCV E1 polypeptide, an HCV E2 polypeptide, or an HCV E1/E2 heterodimer. The present disclosure provides immunogenic compositions comprising: a) one or more adjuvants; b) a fusion polypeptide comprising two or more TPs; and c) an HCV E1 polypeptide, an HCV E2 polypeptide, or an HCV E1/E2 heterodimer. The one or more adjuvants can be one or more of those listed below. In some cases, the one or more adjuvants include a combination of those listed above (e.g., SLA-SE or SLA-LSQ) and one or more of those listed below.

Suitable adjuvants include, e.g., monophosphoryl lipid A (MPL), 3-Q-desacyl-4′-monophosphoryl lipid A (3DMPL), poly(D,L-lactide-co-glycolide) (PLG), 3M-052, MF59, AS03, AS04, AS01, a CpG-containing nucleic acid (where the cytosine is unmethylated), aluminum phosphate, aluminum hydroxide, alum, and combinations thereof. MF59 is 4.3% w/v squalene, 0.5% w/v Tween 80™, 0.5% w/v Span 85. In some cases, the adjuvant is aluminum hydroxide. In some cases, the adjuvant is alum+MPL. In some cases, the adjuvant is MF59. In some cases, the adjuvant is alum+MF59. In some cases, the adjuvant is AS01. AS01 contains QS-21 Stimulon® adjuvant, MPL, and liposomes. In some cases, the adjuvant comprises QS21 and MPL in a liposomal formulation. In some cases, the adjuvant is AS03. A dose of AS03 contains: 10.69 mg squalene; 11.86 mg DL-α-tocopherol; and 4.86 mg polysorbate-80. In some cases, the adjuvant comprises aluminum hydroxide and MPL. In some cases, the adjuvant is AS04. AS04 comprises aluminum hydroxide and MPL. In some cases, the adjuvant is AS15. AS15 is a combination of QS-21 Stimulon® adjuvant, monophosphoryl lipid A, and CpG7909 (an oligonucleotide of the sequence 5′-TCGTCGTTTTGTCGTTTTGTCGTT-3′; (SEQ ID NO:71), in a liposomal formulation. In some instances, the adjuvant is a cyclic dinucleotide (CDN). In some cases, the adjuvant is CpG 1018 (an oligonucleotide of the sequence: 5′ TGACTGTGAACGTTCGAGATGA 3′ (SEQ ID NO:72)).

In some cases, a CDN suitable for use in an immunogenic composition of the present disclosure is of Formula (I):

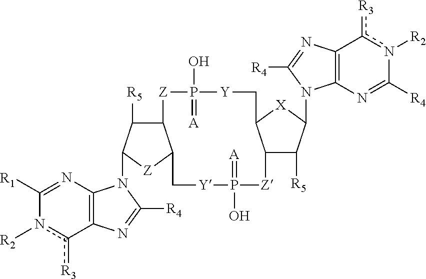

wherein:
A is S or O;
X is S, N, O, CH₂;
Y, Y′ is NH, CH₂, O;
Z, Z′ is NH, CH₂, O;
R1 represents hydrogen or NH₂ which may be substituted;
R2 is hydrogen or absent;
R3 represents NH₂, O, OH, H, or a halogen;
R4 represents hydrogen, halogen, or a straight or branched C₁-C₆ alkyl group which may optionally be substituted;
R5 represents hydrogen, OH or a straight or branched C₁-C₆ alkyl chain or C₁-C₆ straight or branched alkoxy chain which may optionally be substituted;
[symbol] is a single or double bond;
or conjugates thereof, and salts or solvates thereof. See, e.g., US 2008/0286296.

In formula (I), the purine residue is in some cases a guanine (G), adenine (A), xanthine or hypoxanthine (X), or inosine (I) residue. The compound can have identical purine residues, e.g. c-diGMP, c-diAMP, c-diIMP, or c-dXMP, or can contain different purine residues, e.g. c-GpAp, c-GpIp, c-GpXp, c-ApIp, c-ApXp, or c-IpXp. Further, R5 is in some cases an OH group. In addition, X is in some cases an oxygen atom. In one embodiment, Y, Y′, Z, and Z′ are an oxygen atom, O. Thus, in one embodiment, the compound of formula (I) is a cyclic bis(3′-5′)diguanylic acid (c-diGMP) or conjugates thereof or a cyclic bis(3′-5′)diadenylic acid (c-diAMP) or conjugates thereof, or salts or solvates thereof. In one embodiment, the compound of formula (I) is cyclic Bis(3′-5′)adenylic acid, which is also referred to as c-di-AMP; or the pegylated conjugate. With the term “which may be substituted” is meant the substitution with a straight or branched C1-C6 alkyl group or a straight or branched C1-C6 alkoxy group and/or with a halogen, hydroxyl group or carboxyl group.

In some cases, a CDN suitable for use in an immunogenic composition of the present disclosure is selected from the group consisting of cyclic di-adenosine monophosphate (c-di-AMP), cyclic di-guanosine monophosphate (c-di-GMP), and cyclic guanosine monophosphate-adenosine monophosphate (cGAMP). In some cases, a CDN suitable for use in an immunogenic composition of the present disclosure is cGAMP (2′-3′-cyclic GMP-AMP) or cGAMP (3′-3′-cyclic GMP-AMP). In some cases, a CDN suitable for use in an immunogenic composition of the present disclosure is cGAMP (2′-3′-cyclic GMP-AMP). In some cases, a CDN suitable for use in an immunogenic composition of the present disclosure is cGAMP (3′-3′-cyclic GMP-AMP).

In some cases, a CDN suitable for use in an immunogenic composition of the present disclosure is of Formula (II):

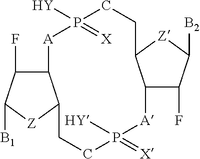

where:
A, C, A′ and C′ are independently selected from NH, O, and S;
X, Y, X′, and Y′ are independently selected from O or S;
Z and Z′ are independently selected from O, S, NH, and CH₂; and
B₁ and B₂ are independently a purine selected from:

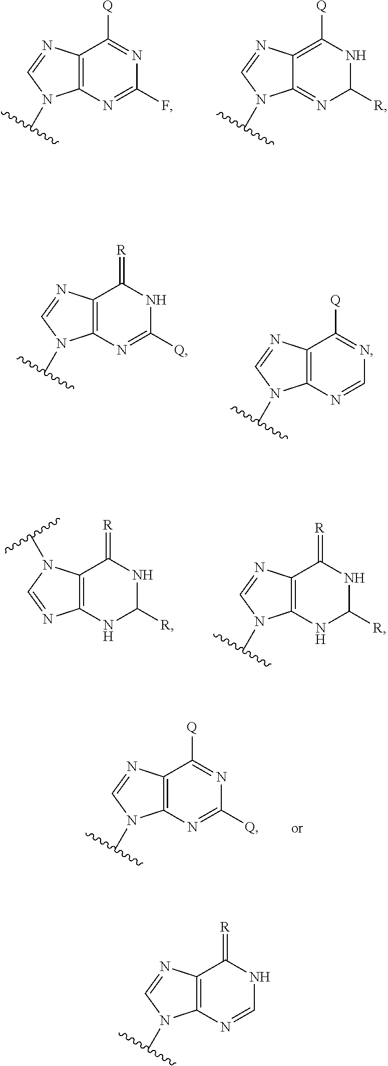

where:
Q is hydrogen or NH₂;
Nitrogen is optionally substituted with a C₁-C₆ alkyl or a C₁-C₆ acyl group; and
R is O or S.

In some cases, a CDN suitable for inclusion in an immunogenic composition of the present disclosure is a fluorinated CDN. In some cases, the fluorinated CDN is 2′-F-c-diGMP having the following structure:

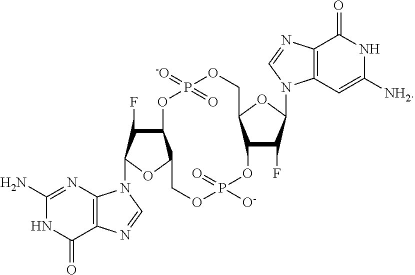

In some cases, an immunogenic composition of the present disclosure comprises: a) an SLA-SE adjuvant, as described above, and a cyclic dinucleotide (CDN); and b) one or more TPs. The present disclosure provides immunogenic compositions comprising: a) an SLA-SE adjuvant, as described above, and a CDN; and b) a fusion polypeptide comprising two or more TPs. The present disclosure provides immunogenic compositions comprising: a) an SLA-SE adjuvant, as described above, and a CDN; b) one or more TPs; and c) an HCV E1 polypeptide, an HCV E2 polypeptide, or an HCV E1/E2 heterodimer. The present disclosure provides immunogenic compositions comprising: a) an SLA-SE adjuvant, as described above, and a CDN; b) a fusion polypeptide comprising two or more TPs; and c) an HCV E1 polypeptide, an HCV E2 polypeptide, or an HCV E1/E2 heterodimer.

In some cases, an immunogenic composition of the present disclosure comprises: a) an SLA-LSQ adjuvant, as described above, and a CDN; and b) one or more TPs. The present disclosure provides immunogenic compositions comprising: a) an SLA-LSQ adjuvant, as described above, and a CDN; and b) a fusion polypeptide comprising two or more TPs. The present disclosure provides immunogenic compositions comprising: a) an SLA-LSQ adjuvant, as described above, and a CDN; b) one or more TPs; and c) an HCV E1 polypeptide, an HCV E2 polypeptide, or an HCV E1/E2 heterodimer. The present disclosure provides immunogenic compositions comprising: a) an SLA-LSQ adjuvant, as described above, and a CDN; b) a fusion polypeptide comprising two or more TPs; and c) an HCV E1 polypeptide, an HCV E2 polypeptide, or an HCV E1/E2 heterodimer.

In some cases, an immunogenic composition of the present disclosure comprises: a) an SLA-SE adjuvant, as described above, and 3M-052; and b) one or more TPs. The present disclosure provides immunogenic compositions comprising: a) an SLA-SE adjuvant, as described above, and 3M-052; and b) a fusion polypeptide comprising two or more TPs. The present disclosure provides immunogenic compositions comprising: a) an SLA-SE adjuvant, as described above, and 3M-052; b) one or more TPs; and c) an HCV E1 polypeptide, an HCV E2 polypeptide, or an HCV E1/E2 heterodimer. The present disclosure provides immunogenic compositions comprising: a) an SLA-SE adjuvant, as described above, and 3M-052; b) a fusion polypeptide comprising two or more TPs; and c) an HCV E1 polypeptide, an HCV E2 polypeptide, or an HCV E1/E2 heterodimer.

In some cases, an immunogenic composition of the present disclosure comprises: a) an SLA-LSQ adjuvant, as described above, and 3M-052; and b) one or more TPs. The present disclosure provides immunogenic compositions comprising: a) an SLA-LSQ adjuvant, as described above, and 3M-052; and b) a fusion polypeptide comprising two or more TPs. The present disclosure provides immunogenic compositions comprising: a) an SLA-LSQ adjuvant, as described above, and 3M-052; b) one or more TPs; and c) an HCV E1 polypeptide, an HCV E2 polypeptide, or an HCV E1/E2 heterodimer. The present disclosure provides immunogenic compositions comprising: a) an SLA-LSQ adjuvant, as described above, and 3M-052; b) a fusion polypeptide comprising two or more TPs; and c) an HCV E1 polypeptide, an HCV E2 polypeptide, or an HCV E1/E2 heterodimer.

In some cases, an immunogenic composition of the present disclosure comprises: a) an SLA-SE adjuvant, as described above, and a CpG oligonucleotide (e.g., CpG 1018); and b) one or more TPs. The present disclosure provides immunogenic compositions comprising: a) an SLA-SE adjuvant, as described above, and a CpG oligonucleotide (e.g., CpG 1018); and b) a fusion polypeptide comprising two or more TPs. The present disclosure provides immunogenic compositions comprising: a) an SLA-SE adjuvant, as described above, and a CpG oligonucleotide (e.g., CpG 1018); b) one or more TPs; and c) an HCV E1 polypeptide, an HCV E2 polypeptide, or an HCV E1/E2 heterodimer. The present disclosure provides immunogenic compositions comprising: a) an SLA-SE adjuvant, as described above, and a CpG oligonucleotide (e.g., CpG 1018); b) a fusion polypeptide comprising two or more TPs; and c) an HCV E1 polypeptide, an HCV E2 polypeptide, or an HCV E1/E2 heterodimer.

In some cases, an immunogenic composition of the present disclosure comprises: a) an SLA-LSQ adjuvant, as described above, and a CpG oligonucleotide (e.g., CpG 1018); and b) one or more TPs. The present disclosure provides immunogenic compositions comprising: a) an SLA-LSQ adjuvant, as described above, and a CpG oligonucleotide (e.g., CpG 1018); and b) a fusion polypeptide comprising two or more TPs. The present disclosure provides immunogenic compositions comprising: a) an SLA-LSQ adjuvant, as described above, and a CpG oligonucleotide (e.g., CpG 1018); b) one or more TPs; and c) an HCV E1 polypeptide, an HCV E2 polypeptide, or an HCV E1/E2 heterodimer. The present disclosure provides immunogenic compositions comprising: a) an SLA-LSQ adjuvant, as described above, and a CpG oligonucleotide (e.g., CpG 1018); b) a fusion polypeptide comprising two or more TPs; and c) an HCV E1 polypeptide, an HCV E2 polypeptide, or an HCV E1/E2 heterodimer.

In some cases, an immunogenic composition of the present disclosure comprises: a) an SLA-SE adjuvant, as described above, and AS03; and b) one or more TPs. The present disclosure provides immunogenic compositions comprising: a) an SLA-SE adjuvant, as described above, and AS03; and b) a fusion polypeptide comprising two or more TPs. The present disclosure provides immunogenic compositions comprising: a) an SLA-SE adjuvant, as described above, and AS03; b) one or more TPs; and c) an HCV E1 polypeptide, an HCV E2 polypeptide, or an HCV E1/E2 heterodimer. The present disclosure provides immunogenic compositions comprising: a) an SLA-SE adjuvant, as described above, and AS03; b) a fusion polypeptide comprising two or more TPs; and c) an HCV E1 polypeptide, an HCV E2 polypeptide, or an HCV E1/E2 heterodimer.

In some cases, an immunogenic composition of the present disclosure comprises: a) an SLA-LSQ adjuvant, as described above, and AS03; and b) one or more TPs. The present disclosure provides immunogenic compositions comprising: a) an SLA-LSQ adjuvant, as described above, and AS03; and b) a fusion polypeptide comprising two or more TPs. The present disclosure provides immunogenic compositions comprising: a) an SLA-LSQ adjuvant, as described above, and AS03; b) one or more TPs; and c) an HCV E1 polypeptide, an HCV E2 polypeptide, or an HCV E1/E2 heterodimer. The present disclosure provides immunogenic compositions comprising: a) an SLA-LSQ adjuvant, as described above, and AS03; b) a fusion polypeptide comprising two or more TPs; and c) an HCV E1 polypeptide, an HCV E2 polypeptide, or an HCV E1/E2 heterodimer.

### Compositions Comprising T-Cell Epitope Polypeptides

As noted above, an immunogenic composition of the present disclosure comprises: a) one or more T-cell epitope polypeptides (also referred to herein as a “TP”); or b) a fusion polypeptide comprising two or more TPs.

Suitable TPs include: i) a TP35-NS3 polypeptide; ii) a TP50C polypeptide; iii) a TP23 polypeptide; iv) a TP27 polypeptide; v) a TP35-NS4 polypeptide; vi) a TP42 polypeptide; vii) a TP45 polypeptide; viii) a TP48 polypeptide; ix) a TP33 polypeptide; x) a TP42-2 polypeptide; xi) a TP65 polypeptide; xii) a TP240 polypeptide; xiii) a TP156 polypeptide; xiv) a TP465 polypeptide; xv) a TP29 polypeptide; xvi) a TP50-NS2 polypeptide; xvii) a TP52 polypeptide; xviii) a TP70 polypeptide; xix) a TP100 polypeptide; xx) a TP171 polypeptide; xxi) TP228 polypeptide; xxii) a TP553 polypeptide; xxiii) a TP778 polypeptide; and xxiv) a TP1987 polypeptide.

Suitable TPs include a TP35-NS3 polypeptide, a TP50C polypeptide, a TP27 polypeptide, a TP48 polypeptide, a TP65 polypeptide, and a TP240 polypeptide, as described below. Suitable TPs include a TP35-NS3 polypeptide, a TP50C polypeptide, a TP27 polypeptide, a TP48 polypeptide, a TP65 polypeptide, a TP240 polypeptide, a TP156 polypeptide, and a TP465 polypeptide, as described below.

In some cases, a TP is from HCV of genotype 1. In some cases, a TP is from HCV of genotype 1a. In some cases, a TP is from HCV of genotype 1b. In some cases, a TP is from HCV of genotype 2. In some cases, a TP is from HCV of genotype 2a. In some cases, a TP is from HCV of genotype 2b. In some cases, a TP is from HCV of genotype 3. In some cases, a TP is from HCV of genotype 3a. In some cases, a TP is from HCV of genotype 4. In some cases, a TP is from HCV of genotype 5. In some cases, a TP is from HCV of genotype 6. In some cases, a TP is from HCV of genotype 7. In some cases, a composition of the present disclosure comprises TPs of HCV of two or more different genotypes.

### TP35-NS3 Polypeptides

In some cases, a TP35-NS3 T-cell epitope polypeptide (a “TP35-NS3 polypeptide”) comprises the amino acid sequence: KSTKVPX₁AYX₂X₃QGYX₄VLVLNPSVAATLGFGX₅X₆X₇SX₈ (SEQ ID NO:73), wherein X₁ is A or V; X₂ is A or V; X₃ is A or S; X₄ is K or N; X₅ is A or S; X₆ is Y or F; X₇ is M or L; and X₈ is K or R, where the TP35-NS3 T-cell epitope polypeptide has a length of from 35 amino acids to 40 amino acids (e.g., 35, 36, 37, 38, 39, or 40 amino acids). In some cases, a TP35-NS3 polypeptide has a length of 35 amino acids.

Non-limiting examples of TP35-NS3 polypeptides include:

i)
(SEQ ID NO: 74)
KSTKVPVAYAAQGYKVLVLNPSVAATLGFGAYLSK;
ii)
(SEQ ID NO: 75)
KSTKVPAAYASQGYKVLVLNPSVAATLGFGAYMSK;
iii)
(SEQ ID NO: 76)
KSTKVPAAYVAQGYNVLVLNPSVAATLGFGSFMSR;
iv)
(SEQ ID NO: 77)
KSTKVPAAYAAQGYKVLVLNPSVAATLGFGAYMSR;
v)
(SEQ ID NO: 78)
KSTKVPAAYASQGYKVLVLNPSVAATLGFGSYMSK;
and
vi)
(SEQ ID NO: 75)
KSTKVPAAYASQGYKVLVLNPSVAATLGFGAYMSK

In some cases, a TP35-NS3 polypeptide comprises an amino acid sequence having at least 80% (at least 80%, at least 85%, at least 90%, least 95%, least 98%, least 99%, or 100%) amino acid sequence identity to the amino acid sequence: KSTKVPAAYAAQGYKVLVLNPSVAATLGFGAYMSK (SEQ ID NO:79), where the TP35-NS3 polypeptide has a length of from 35 amino acids to 40 amino acids (e.g., 35, 36, 37, 38, 39, or 40 amino acids). In some cases, a TP35-NS3 polypeptide comprises the amino acid sequence KSTKVPAAYAAQGYKVLVLNPSVAATLGFGAYMSK (SEQ ID NO:79) and has a length of 35 amino acids.

### TP5C Polypeptides

In some cases, a TP50C T-cell epitope polypeptide (a “TP50C polypeptide”) comprises the amino acid sequence: GVYX₁LPRRGPRLGVRX₂TRKX₃SERSQPRGRRQX₄IPKX₅XX₇XX₉GX₁₀X₁WX₁₂X₁₃PGYP (SEQ ID NO:80), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, where the TP50C T-cell epitope polypeptide has a length of from 50 amino acids to 60 amino acids (e.g., 50, 51, 52, 53, 54, 55, 56, 57, 58, 59, or 60 amino acids). In some cases, a TP50C T-cell epitope polypeptide has a length of 50 amino acids.

In some cases, a TP50-C T-cell epitope polypeptide comprises the following amino acid sequence: GVYX₁LPRRGPRLGVRX₂TRKX₃SERSQPRGRRQX₄IPKX₅XX₇XX₉GX₁₀X₁WX₁₂X₁₃PGYP (SEQ ID NO:80), where X₁ is L or V; X₂ is A; X₃ is T; X₄ is P; X₅ is A or D; X₆ is R; X₇ is R or Q; X₈ is S or P; X₉ is E or T; X₁₀ is R or K; X₁₁ is S, T, H, or A; X₁₂ is A or G; and X₁₃ is Q or K; where the TP50C T-cell epitope polypeptide has a length of from 50 amino acids to 60 amino acids (e.g., 50, 51, 52, 53, 54, 55, 56, 57, 58, 59, or 60 amino acids). In some cases, a TP50C T-cell epitope polypeptide has a length of 50 amino acids.

Non-limiting examples of TP50C polypeptides include:

(SEQ ID NO: 81)
GVYLLPRRGPRLGVRATRKTSERSQPRGRRQPIPKARRPEGRTWAQPG
YP;
ii)
(SEQ ID NO: 82)
GVYLLPRRGPRLGVRATRKTSERSQPRGRRQPIPKDRRSTGKSWGKPG
YP;
iii)
(SEQ ID NO: 83)
GVYVLPRRGPRLGVRATRKTSERSQPRGRRQPIPKARRSEGRSWAQPG
YP;
iv)
(SEQ ID NO: 84)
GVYLLPRRGPRLGVRATRKTSERSQPRGRRQPIPKARRPTGRSWGQPG
YP;
v)
(SEQ ID NO: 85)
GVYLLPRRGPRLGVRATRKTSERSQPRGRRQPIPKARQPTGRHWAQPG
YP;
and
vi)
(SEQ ID NO: 86)
GVYLLPRRGPRLGVRTTRKSSERSQPRGRRQRIPKAASSQGKAWGKPG
YP.

In some cases, a TP50C polypeptide comprises an amino acid sequence having at least 80% (at least 80%, at least 85%, at least 90%, least 95%, least 98%, least 99%, or 100%) amino acid sequence identity to the amino acid sequence: GVYLLPRRGPRLGVRATRKTSERSQPRGRRQPIPKARRSEGRSWAQPGYP (SEQ ID NO:87), where the TP50C T-cell epitope polypeptide has a length of from 50 amino acids to 60 amino acids (e.g., 50, 51, 52, 53, 54, 55, 56, 57, 58, 59, or 60 amino acids). In some cases, a TP50C polypeptide comprises the amino acid sequence GVYLLPRRGPRLGVRATRKTSERSQPRGRRQPIPKARRSEGRSWAQPGYP (SEQ ID NO:87), and has a length of 50 amino acids.

### TP23 Polypeptides

In some cases, a TP23 polypeptide comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: DVVVVATDALMTGFTGDFDSVID (SEQ ID NO:88; also referred to herein as “TP23”); where the TP has a length of from 18 amino acids to 23 amino acids (e.g., the TP has a length of 18 amino acids (aa), 19 aa, 20 aa, 21 aa, 22 aa, or 23 aa). In some cases, the TP has a length of 23 amino acids.

In some cases, a TP23 polypeptide comprises the following amino acid sequence: DVVVX₁X₂TDALMTGX₃TGDFDSVID (SEQ ID NO:89), where X₁ is V or C; X₂ is A or S; and X₃ is F or Y; where the TP23 T-cell epitope polypeptide has a length of from 18 amino acids to 23 amino acids (e.g., the TP has a length of 18 amino acids (aa), 19 aa, 20 aa, 21 aa, 22 aa, or 23 aa). In some cases, the TP has a length of 23 amino acids.

In some cases, a TP23 polypeptide comprises the following amino acid sequence: DVVVVATDALMTGYTGDFDSVID (SEQ ID NO:90); where the TP23 polypeptide has a length of from 18 amino acids to 23 amino acids (e.g., the TP has a length of 18 amino acids (aa), 19 aa, 20 aa, 21 aa, 22 aa, or 23 aa). In some cases, the TP has a length of 23 amino acids. In some cases, the TP is part of a fusion polypeptide, as described in detail below.

In some cases, a TP23 polypeptide comprises the following amino acid sequence: DVVVCATDALMTGFTGDFDSVID (SEQ ID NO:91); where the TP23 T-cell epitope polypeptide has a length of from 18 amino acids to 23 amino acids (e.g., T-cell epitope polypeptide has a length of 18 amino acids (aa), 19 aa, 20 aa, 21 aa, 22 aa, or 23 aa). In some cases, the TP has a length of 23 amino acids. In some cases, the TP is part of a fusion polypeptide, as described in detail below.

In some cases, a TP23 polypeptide comprises the following amino acid sequence: DVVVCSTDALMTGFTGDFDSVID (SEQ ID NO:92); where the TP23 polypeptide has a length of from 18 amino acids to 23 amino acids (e.g., the TP has a length of 18 amino acids (aa), 19 aa, 20 aa, 21 aa, 22 aa, or 23 aa). In some cases, the TP has a length of 23 amino acids. In some cases, the TP is part of a fusion polypeptide, as described in detail below.

In some cases, a TP23 polypeptide comprises the following amino acid sequence: DVVVCATDALMTGYTGDFDSVID (SEQ ID NO:93); where the TP23 T-cell epitope polypeptide has a length of from 18 amino acids to 23 amino acids (e.g., T-cell epitope polypeptide has a length of 18 amino acids (aa), 19 aa, 20 aa, 21 aa, 22 aa, or 23 aa). In some cases, the TP23 T-cell epitope polypeptide has a length of 23 amino acids. In some cases, the TP23 T-cell epitope polypeptide is part of a fusion polypeptide, as described in detail below.

In some cases, a TP23 polypeptide comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: DVVVVATDALMTGFTGDFDSVID (SEQ ID NO:88; “TP23”); where the TP23 polypeptide has a length of from 23 amino acids to 30 amino acids (e.g., T-cell epitope polypeptide has a length of 23 amino acids (aa), 24 aa, 25 aa, 26 aa, 27 aa, 28 aa, 29 aa, or 30 aa).

In some cases, a TP23 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: DVVVVATDALMTGFTGDFDSVID (SEQ ID NO:88); and has a length of 23 amino acids. In some cases, a TP23 polypeptide comprises the amino acid sequence DVVVVATDALMTGFTGDFDSVID (SEQ ID NO:88); and has a length of 23 amino acids.

### TP27 Polypeptides

In some cases, a TP27 T-cell epitope polypeptide (a “TP27 polypeptide”) comprises the following amino acid sequence: X₁X₂X₃X₄KGGRHLIFCHSKKKCDEX₅AX₆X₇LX₈ (SEQ ID NO:94), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, where the TP27 T-cell epitope polypeptide has a length of from 27 amino acids to 32 amino acids (e.g., 27, 28, 29, 30, 31, or 32 amino acids). In some cases, a TP27 polypeptide has a length of 27 amino acids.

Non-limiting examples of TP27 polypeptides include:

(SEQ ID NO: 95)
LEVIKGGRHLIFCHSKKKCDELAAKLV;
ii)
(SEQ ID NO: 96)
IETIKGGRHLIFCHSKKKCDELAAKLS;
iii)
(SEQ ID NO: 97)
LSYIKGGRHLIFCHSKKKCDELAAALR;
iv)
(SEQ ID NO: 98)
LAFIKGGRHLIFCHSKKKCDELAALR;
v)
(SEQ ID NO: 99)
IAQLKGGRHLIFCHSKKKCDEIASKLR;
vi)
(SEQ ID NO: 100)
LELIKGGRHLIFCHSKKKCDELAQLT;
vii)
(SEQ ID NO: 101)
LEYIKGGRHLIFCHSKKKCDELAKOLT;
viii)
(SEQ ID NO: 102)
LQHIKGGRHLIFCHSKKKCDELAGKLT;
and
ix)
(SEQ ID NO: 103)
LEQIKGGRHLIFCHSKKKCDELAAKLR.

In some cases, a TP27 polypeptide comprises an amino acid sequence having at least 80% (at least 80%, at least 85%, at least 90%, least 95%, least 98%, least 99%, or 100%) amino acid sequence identity to the amino acid sequence: LEQIKGGRHLIFCHSKKKCDELAAKLR (SEQ ID NO:103), where the TP27 polypeptide has a length of from 27 amino acids to 32 amino acids (e.g., 27, 28, 29, 30, 31, or 32 amino acids). In some cases, a TP27 polypeptide has a length of 27 amino acids. In some cases, a TP27 polypeptide comprises the amino acid sequence LEQIKGGRHLIFCHSKKKCDELAAKLR (SEQ ID NO:103) and has a length of 27 amino acids.

### TP35-NS4 Polypeptides

In some cases, TP35-NS4 polypeptide comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: ILRRHVGPGEGAVQWMNRLIAFASRGNHVSPTHYV (SEQ ID NO:104; also referred to herein as “TP35-NS4”); where the T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the T-cell epitope polypeptide is part of a fusion polypeptide, as described in detail below.

In some cases, a TP35-NS4 polypeptide comprises the following amino acid sequence: X₁X₂X₃RHX₄GX₅X₆EGX₇X₈QWMNRLIAFASRGNHVX₉PTHYX₁₀ (SEQ ID NO:105), where X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; and X₁₀ is V or I; where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

In some cases, a TP35-NS4 polypeptide comprises the following amino acid sequence: X₁X₂X₃RHX₄GX₅X₆EGX₇X₈QWMNRLIAFASRGNHVX₉PTHYX₁₀ (SEQ ID NO:105), where X₁ is I or V; X₂ is L; X₃ is R; X₄ is V or I; X₅ is P or Q; X₆ is G or A; X₇ is A; X₈ is V or T; X₉ is S or A; and X₁₀ is V; where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

In some cases, a TP35-NS4 polypeptide comprises the following amino acid sequence: ILRRHVGPGEGAVQWMNRLIAFASRGNHVAPTHYV (SEQ ID NO:106); where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

In some cases, a TP35-NS4 polypeptide comprises the following amino acid sequence: ILRRHVGQGEGAVQWMNRLIAFASRGNHVAPTHYV (SEQ ID NO:107); where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

In some cases, a TP35-NS4 polypeptide comprises the following amino acid sequence: VLRRHIGPGEGAVQWMNRLIAFASRGNHVSPTHYV (SEQ ID NO:108); where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

In some cases, a TP35-NS4 polypeptide comprises the following amino acid sequence: ILRRHVGPAEGATQWMNRLIAFASRGNHVSPTHYV (SEQ ID NO:109); where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

In some cases, a TP35-NS4 polypeptide comprises the following amino acid sequence: IIKRHTGTSEGVTQWMNRLIAFASRGNHVSPTHYI (SEQ ID NO:110); where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the T-cell epitope polypeptide is part of a fusion polypeptide, as described in detail below.

In some cases, a TP35-NS4 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYV (SEQ ID NO:104); and has a length of from 35 amino acids to 40 amino acids (e.g., 35 amino acids (aa), 36 aa, 37 aa, 38 aa, 39 aa, or 40 aa).

In some cases, a TP35-NS4 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYV (SEQ ID NO:104); and has a length of 35 amino acids. In some cases, a T-cell epitope polypeptide suitable for inclusion in a composition of the present disclosure comprises the amino acid sequence ILRRHVGPGEGAVQWMNRLIAFASRGNHVSPTHYV (SEQ ID NO:104); and has a length of 35 amino acids.

### TP42 Polypeptides

In some cases, a TP42 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GTEGEIPFYGKAIPLEQIKGGRHLIFCHSKKKCDELAAKLTG (SEQ ID NO:111); and has a length of from 33 amino acids to about 50 amino acids (e.g., 33 amino acids (aa), 34 aa, 35 aa, 36 aa, 37 aa, 38 aa, 39 aa, 40 aa, 41 aa, 42 aa, or from 42 aa to 50 aa). In some cases, a TP42 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GTEGEIPFYGKAIPLEQIKGGRHLIFCHSKKKCDELAAKLTG (SEQ ID NO:111); and has a length of 42 amino acids. In some cases, a TP42 polypeptide comprises the amino acid sequence GTEGEIPFYGKAIPLEQIKGGRHLIFCHSKKKCDELAAKLTG (SEQ ID NO:111); and has a length of 42 amino acids.

In some cases, a TP42 polypeptide comprises the following amino acid sequence: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:112), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and where the TP42 T-cell epitope polypeptide has a length of from 34 amino acids to 52 amino acids (e.g., 34 amino acids (aa), 35 aa, 36 aa, 37 aa, 38 aa, 39 aa, 40 aa, 41 aa, 42 aa, 43 aa, 44 aa, 45 aa, 46 aa, 47 aa, 48 aa, 49 aa, 50 aa, 51 aa, or 52 aa. In some cases, a TP42 T-cell epitope polypeptide suitable for inclusion in a composition of the present disclosure comprises the following amino acid sequence: X₁X₂X₃GEIPFYGX₄AIPX₅X₆X₇X₈KGGRHLIFCHSKKKCDEX₉AX₁₀X₁₁LX₁₂X₁₃KKK (SEQ ID NO:112), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S. In some cases, a TP42 polypeptide comprises the following amino acid sequence: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:112), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, V, or L; X₈ is I or L; X₉ is L or I; X₁₀ is A, K, or S; X₁₁ is K, Q, or A; X₁₂ is T, R, V, or S; and X₁₃ is G or S, wherein n is an integer from 2 to 10, and where the TP42 T-cell epitope polypeptide has a length of from 34 amino acids to 52 amino acids (e.g., 34 amino acids (aa), 35 aa, 36 aa, 37 aa, 38 aa, 39 aa, 40 aa, 41 aa, 42 aa, 43 aa, 44 aa, 45 aa, 46 aa, 47 aa, 48 aa, 49 aa, 50 aa, 51 aa, or 52 aa. In some cases, a TP42 polypeptide comprises the following amino acid sequence: X₁X₂X₃GEIPFYGX₄AIPX₅X₆X₇X₈KGGRHLIFCHSKKKCDEX₉AX₁₀X₁LX₁₂X₁₃KKK (SEQ ID NO:112), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, V, or L; X₈ is I or L; X₉ is L or I; X₁₀ is A, K, or S; X₁₁ is K, Q, or A; X₁₂ is T, R, V, or S; and X₁₃ is G or S. In some cases, the TP42 T-cell epitope polypeptide has a length of 45 amino acids. In some cases, the TP42 T-cell epitope polypeptide comprises the following amino acid sequence: GTEGEIPFYGKAIPLEQIKGGRHLIFCHSKKKCDELAAKLTGKKK (SEQ ID NO:114) and has a length of 45 amino acids.

In some cases, a TP42 polypeptide comprises the following amino acid sequence:

(SEQ ID NO: 112)
X₁X₂X₃GEIPFYGX₄AIPX₅X₆X₇X₈KGGRHLIFCHSKKKCDEX₉AX₁₀X₁₁
LX₁₂X₁₃ ,

where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S; where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 polypeptide comprises the following amino acid sequence: STTGEIPFYGKAIPLEVIKGGRHLIFCHSKKKCDELAAKLVA (SEQ ID NO:115); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 polypeptide comprises the following amino acid sequence: SNTGEIPFYGKAIPIETIKGGRHLIFCHSKKKCDELAAKLSG (SEQ ID NO:116); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 polypeptide comprises the following amino acid sequence: GQEGEIPFYGRAIPLSYIKGGRHLIFCHSKKKCDELAAALRG (SEQ ID NO:117); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 polypeptide comprises the following amino acid sequence: GHEGEIPFYGKAIPLAFIKGGRHLIFCHSKKKCDELAALRG (SEQ ID NO: 118); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 polypeptide comprises the following amino acid sequence: GSEGEIPFYGKAIPIAQLKGGRHLIFCHSKKKCDEIASKLRG (SEQ ID NO:119); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 T-cell epitope polypeptide suitable for inclusion in a composition of the present disclosure comprises the following amino acid sequence: PTTGEIPFYGKAIPLELIKGGRHLIFCHSKKKCDELAQLTS (SEQ ID NO:120); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 polypeptide comprises the following amino acid sequence: PSEGEIPFYGRAIPLXLIKGGRHLIFCHSKKKCDELAKQLTS (SEQ ID NO:121); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a TP42 polypeptide comprises the following amino acid sequence: PTTGEIPFYGKAIPLEYIKGGRHLIFCHSKKKCDELAKQLTS (SEQ ID NO:122); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, TP42 polypeptide has a length of 42 amino acids. In some cases, the T-cell epitope polypeptide is part of a fusion polypeptide, as described in detail below.

In some cases, a TP42 polypeptide comprises the following amino acid sequence: GNDGEIPFYGKAIPLQHIKGGRHLIFCHSKKKCDELAGKLTS (SEQ ID NO:123); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the T-cell epitope polypeptide has a length of 42 amino acids. In some cases, the T-cell epitope polypeptide is part of a fusion polypeptide, as described in detail below.

In some cases, a suitable T-cell epitope polypeptide is a TP42 polypeptide with from 1 amino acid to 5 amino acids removed from the N-terminus and/or from 1 amino acid to 5 amino acids removed from the C-terminus. Thus, e.g., a suitable T-cell epitope polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: IPFYGKAIPLEQIKGGRHLIFCHSKKKCDELAAKLTG (SEQ ID NO:124); and has a length of 37 amino acids. As another example, a suitable T-cell epitope polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GTEGEIPFYGKAIPLEQIKGGRHLIFCHSKKKCDELA (SEQ ID NO:125); and has a length of 37 amino acids.

### TP45 Polypeptides

In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVIDCNV (SEQ ID NO:127); and has a length of from 45 amino acids to about 50 amino acids. In some cases, a T-cell epitope polypeptide suitable for inclusion in a composition of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVIDCNV (SEQ ID NO:127); and has a length of 45 amino acids. In some cases, a T-cell epitope polypeptide suitable for inclusion in a composition of the present disclosure comprises the amino acid sequence LNAVAYYRGLDVSVIPTSGDVVVVATDALMTGFTGDFDSVIDCNV (SEQ ID NO:127); and has a length of 45 amino acids. In some cases, a T-cell epitope polypeptide suitable for inclusion in a composition of the present disclosure comprises the amino acid sequence LNAVAYYRGLDVSVIPTSGDVVVVATDALMTGFTGDFDSVIDKKK (SEQ ID NO:128); and has a length of 45 amino acids.

In some cases, a TP45 polypeptide comprises the following amino acid sequence: X₁X₂AVAX₃YRGX₄DVX₅X₆IPX₇X₈GDVVVX₉X₁₀TDALMTGX₁₁TGDFDSVIDX₁₂X₁₃ X₁₄(K)n (SEQ ID NO:129), where X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K, wherein n is an integer from 2 to 10, and where the TP45 T-cell epitope polypeptide has a length of from 36 amino acids to 55 amino acids. In some cases, a TP45 polypeptide comprises the following amino acid sequence: X₁X₂AVAX₃YRGX₄DVX₅X₆IPX₇X₈GDVVVX₉X₁₀TDALMTGX₁₁TGDFDSVIDX₁₂X₁₃ X₁₄KKK (SEQ ID NO:129), where X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K; where the TP45 T-cell epitope polypeptide has a length of 48 amino acids. In some cases, the TP45 T-cell epitope polypeptide comprises the following amino acid sequence: LNAVAYYRGLDVSVIPTSGDVVVVATDALMTGFTGDFDSVIDCNVKKK (SEQ ID NO:131) and has a length of 48 amino acids.

In some cases, a TP45 polypeptide comprises the following amino acid sequence: X₁X₂AVAX₃YRGX₄DVX₅X₆IPX₇X₈GDVVVX₉X₁₀TDALMTGX₁₁TGDFDSVIDX₁₂X₁₃ X₁₄ (SEQ ID NO:129), where X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K; and where the TP45 T-cell epitope polypeptide has a length of from 36 amino acids to 55 amino acids. In some cases, the TP45 T-cell epitope polypeptide has a length of 45 amino acids. In some cases, the TP45 T-cell epitope polypeptide is part of a fusion polypeptide, as described in detail below.

In some cases, a TP45 polypeptide comprises the following amino acid sequence: X₁X₂AVAX₃YRGX₄DVX₅X₆IPX₇X₈GDVVVX₉X₁₀TDALMTGX₁₁TGDFDSVIDX₁₂X₁₃ X₁₄ (SEQ ID NO:129), where X₁ is L; X₂ is N; X₃ is Y; X₄ is L; X₅ is S; X₆ is V; X₇ is T; X₈ is S, Q, or T; X₉ is V or C; X₁₀ is A; X₁₁ is F or Y; X₁₂ is K; X₁₃ is K; and X₁₄ is K; and where the TP45 T-cell epitope polypeptide has a length of from 36 amino acids to 55 amino acids. In some cases, the TP45 T-cell epitope polypeptide has a length of 45 amino acids.

In some cases, a suitable T-cell epitope polypeptide is a TP45 polypeptide with from 1 amino acid to 5 amino acids removed from the N-terminus and/or from 1 amino acid to 5 amino acids removed from the C-terminus. Thus, for example, in some cases, a TP45 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVID (SEQ ID NO:133); and has a length of 43 amino acids. For example, in some cases, a TP45 polypeptide comprises the amino acid sequence LNAVAYYRGLDVSVIPTSGDVVVVATDALMTGFTGDFDSVID (SEQ ID NO:133); and has a length of 43 amino acids.

### TP48 Polypeptides

In some cases, a TP48 T-cell epitope polypeptide (a “TP48 polypeptide”) comprises the following amino acid sequence: X₁X₂X₃RHX₄GX₅X₆EGX₇X₈QWMNRLIAFASRGNHVX₉PTHYX₁₀X₁₁X₁₂X₃DAX₁₄X₁₅X₁₆VX₁₇X₁₈X₁₉ L (SEQ ID NO:134), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁s is A, Q, R, or K; X₁₆ is R, K, or X; X₁₇ is T or M; X₁₈ is Q, A, T, or G; and X₁₉ is I, L, or V, where the TP48 T-cell epitope polypeptide has a length of from 48 amino acids to 53 amino acids (e.g., 48, 49, 50, 51, 52, or 53 amino acids). In some cases, a TP48 polypeptide has a length of 48 amino acids.

Non-limiting examples of TP48 polypeptides include:

(SEQ ID NO: 135)
ILRRHVGPGEGAVQWMNRLIAFASRGNHVSPTHYVPESDAAARVTAIL;
ii)
(SEQ ID NO: 136)
ILRRHVGPGEGAVQWMNRLIAFASRGNHVSPTHYVPESDAAARVTQIL;
iii)
(SEQ ID NO: 137)
ILRRHVGPGEGAVQWMNRLIAFASRGNHVAPTHYVTESDASQRVTQLL;
iv)
(SEQ ID NO: 138)
ILRRHVGQGEGAVQWMNRLIAFASRGNHVAPTHYVAESDASQRVTQVL;
v)
(SEQ ID NO: 139)
ILRRHVGPGEGAVQWMNRLIAFASRGNHVSPTHYVPESDAAARVTALL;
vi)
(SEQ ID NO: 136)
ILRRHVGPGEGAVQWMNRLIAFASRGNHVSPTHYVPESDAAARVTQIL;
vii)
(SEQ ID NO: 140)
VLRRHIGPGEGAVQWMNRLIAFASRGNHVSPTHYVPETDASAKVTQLL;
viii)
(SEQ ID NO: 141)
IIKRHTGTSEGVTQWMNRLIAFASRGNHVSPTHYIQDDDASKRVMGIL;
and
ix)
(SEQ ID NO: 136)
ILRRHVGPGEGAVQWMNRLIAFASRGNHVSPTHYVPESDAAARVTQIL.

In some cases, a TP48 polypeptide comprises an amino acid sequence having at least 80% (at least 80%, at least 85%, at least 90%, least 95%, least 98%, least 99%, or 100%) amino acid sequence identity to the amino acid sequence: ILRRHVGPGEGAVQWMNRLIAFASRGNHVSPTHYVPESDAAARVTQIL (SEQ ID NO:136), where the TP48 T-cell epitope polypeptide has a length of from 48 amino acids to 53 amino acids (e.g., 48, 49, 50, 51, 52, or 53 amino acids). In some cases, a TP48 polypeptide has a length of 48 amino acids. In some cases, a TP48 polypeptide comprises the amino acid sequence: ILRRHVGPGEGAVQWMNRLIAFASRGNHVSPTHYVPESDAAARVTQIL (SEQ ID NO:136) and has a length of 48 amino acids.

### TP33 Polypeptides

In some cases, a TP33 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: HSKKKCDELAAKLTGLGLNAVAYYRGLDVSVIP (SEQ ID NO:142); and has a length of from 33 amino acids to about 37 amino acids (e.g., has a length of 33 amino acids (aa), 34 aa, 35 aa, 36 aa, or 37 aa), or has a length of from 30 amino acids to about 33 amino acids (e.g., has a length of 30 amino acids (aa), 31 aa, 32 aa, or 33 aa). In some cases, a TP33 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: HSKKKCDELAAKLTGLGLNAVAYYRGLDVSVIP (SEQ ID NO:142); and has a length of 33 amino acids. In some cases, a TP33 polypeptide comprises the amino acid sequence HSKKKCDELAAKLTGLGLNAVAYYRGLDVSVIP (SEQ ID NO:142); and has a length of 33 amino acids.

In some cases, a TP33 polypeptide comprises the following amino acid sequence: HSKKKCDELAX₁X₂LX₃X₄X₅GX₆NAVAYYRGLDVSX₇IP (SEQ ID NO:143), where X₁ is A or S; X₂ is K or A; X₃ is V, S, R, or T; X₄ is A or G; X₅ is L or M; X₆ is I, L, or V; and X₇ is V or I; and has a length of from 33 amino acids to about 37 amino acids (e.g., has a length of 33 amino acids (aa), 34 aa, 35 aa, 36 aa, or 37 aa), or has a length of from 30 amino acids to about 33 amino acids (e.g., has a length of 30 amino acids (aa), 31 aa, 32 aa, or 33 aa). In some cases, a TP33 polypeptide comprises the following amino acid sequence: HSKKKCDELAX₁X₂LX₃X₄X₅GX₆NAVAYYRGLDVSX₇IP (SEQ ID NO:143), where X₁ is A or S; X₂ is K or A; X₃ is V, S, R, or T; X₄ is A or G; X₅ is L or M; X₆ is I, L, or V; and X₇ is V or I; where the TP33 T-cell epitope polypeptide has a length of 33 amino acids.

In some cases, a TP33 polypeptide comprises the following amino acid sequence: HSKKKCDELAAKLX₁X₂X₃GX₄NAVAYYRGLDVSVIP (SEQ ID NO:144), where X₁ is V, S, R, or T; X₂ is A or G; X₃ is L or M; and X₄ is I, L, or V; and has a length of from 33 amino acids to about 37 amino acids (e.g., has a length of 33 amino acids (aa), 34 aa, 35 aa, 36 aa, or 37 aa), or has a length of from 30 amino acids to about 33 amino acids (e.g., has a length of 30 amino acids (aa), 31 aa, 32 aa, or 33 aa). In some cases, a TP33 polypeptide comprises the following amino acid sequence: HSKKKCDELAAKLX₁X₂X₃GX₄NAVAYYRGLDVSVIP (SEQ ID NO:144), where X₁ is V, S, R, or T; X₂ is A or G; X₃ is L or M; and X₄ is I, L, or V; where the TP33 T-cell epitope polypeptide has a length of 33 amino acids.

In some cases, a TP33 polypeptide comprises the following amino acid sequence: HSKKKCDELAAALRGMGX₁NAVAYYRGLDVSX₁IP (SEQ ID NO:145), where X₁ is I, L, or V; and X₂ is V or I; and has a length of from 33 amino acids to about 37 amino acids (e.g., has a length of 33 amino acids (aa), 34 aa, 35 aa, 36 aa, or 37 aa), or has a length of from 30 amino acids to about 33 amino acids (e.g., has a length of 30 amino acids (aa), 31 aa, 32 aa, or 33 aa). In some cases, a TP33 polypeptide comprises the following amino acid sequence: HSKKKCDELAAALRGMGX₁NAVAYYRGLDVSX₁IP (SEQ ID NO:145), where X₁ is I, L, or V; and X₂ is V or I; where the TP33 T-cell epitope polypeptide has a length of 33 amino acids.

In some cases, a TP33 polypeptide comprises the amino acid sequence HSKKKCDELAAKLRGMGLNAVAYYRGLDVSVIP (SEQ ID NO:146); and has a length of 33 amino acids.

### TP42-2 Polypeptides

In some cases, a T42-2 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KGGRHLIFCHSKKKCDELAAKLTGLGLNAVAYYRGLDVSVIP (SEQ ID NO:147); and has a length of from 42 amino acids to about 46 amino acids (e.g., has a length of 42 amino acids (aa), 43 aa, 44 aa, 45 aa, or 46 aa), or has a length of from 38 amino acids to about 42 amino acids (e.g., has a length of 38 amino acids (aa), 39 aa, 40 aa, 41 aa, or 42 aa). In some cases, T42-2 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KGGRHLIFCHSKKKCDELAAKLTGLGLNAVAYYRGLDVSVIP (SEQ ID NO:147); and has a length of 42 amino acids. In some cases, a T42-2 polypeptide comprises the amino acid sequence KGGRHLIFCHSKKKCDELAAKLTGLGLNAVAYYRGLDVSVIP (SEQ ID NO:147); and has a length of 42 amino acids.

In some cases, a T42-2 polypeptide comprises the following amino acid sequence: KGGRHLIFCHSKKKCDELAX₁X₂LX₃X₄X₅GX₆NAVAYYRGLDVSX₇IP (SEQ ID NO:148), where X₁ is A or S; X₂ is K or A; X₃ is V, S, R, or T; X₄ is A or G; X₅ is L or M; X₆ is I, L, or V; and X₇ is V or I; and has a length of from 42 amino acids to about 46 amino acids (e.g., has a length of 42 amino acids (aa), 43 aa, 44 aa, 45 aa, or 46 aa), or has a length of from 38 amino acids to about 42 amino acids (e.g., has a length of 38 amino acids (aa), 39 aa, 40 aa, 41 aa, or 42 aa). In some cases, a T42-2 polypeptide comprises the following amino acid sequence: KGGRHLIFCHSKKKCDELAX₁X₂LX₃X₄X₅GX₆NAVAYYRGLDVSX₇IP (SEQ ID NO:148), where X₁ is A or S; X₂ is K or A; X₃ is V, S, R, or T; X₄ is A or G; X₅ is L or M; X₆ is I, L, or V; and X₇ is V or I; where the T42-2 polypeptide has a length of 42 amino acids.

In some cases, a T42-2 polypeptide comprises the following amino acid sequence: KGGRHLIFCHSKKKCDELAAKLX₁X₂X₃GX₄NAVAYYRGLDVSVIP (SEQ ID NO:149), where X₁ is V, S, R, or T; X₂ is A or G; X₃ is L or M; and X₄ is I, L, or V; and has a length of from 42 amino acids to about 46 amino acids (e.g., has a length of 42 amino acids (aa), 43 aa, 44 aa, 45 aa, or 46 aa), or has a length of from 38 amino acids to about 42 amino acids (e.g., has a length of 38 amino acids (aa), 39 aa, 40 aa, 41 aa, or 42 aa). In some cases, a T42-2 polypeptide comprises the following amino acid sequence: KGGRHLIFCHSKKKCDELAAKLX₁X₂X₃GX₄NAVAYYRGLDVSVIP (SEQ ID NO:149), where X₁ is V, S, R, or T; X₂ is A or G; X₃ is L or M; and X₄ is I, L, or V; where T42-2 polypeptide has a length of 42 amino acids.

In some cases, a T42-2 polypeptide comprises the following amino acid sequence: KGGRHLIFCHSKKKCDELAAALRGMGX₁NAVAYYRGLDVSXIIP (SEQ ID NO:150), where X₁ is I, L, or V; and X₂ is V or I; and has a length of from 42 amino acids to about 46 amino acids (e.g., has a length of 42 amino acids (aa), 43 aa, 44 aa, 45 aa, or 46 aa), or has a length of from 38 amino acids to about 42 amino acids (e.g., has a length of 38 amino acids (aa), 39 aa, 40 aa, 41 aa, or 42 aa). In some cases, a T42-2 polypeptide comprises the following amino acid sequence: HSKKKCDELAAALRGMGX₁NAVAYYRGLDVSX₁IP (SEQ ID NO:145), where X₁ is I, L, or V; and X₂ is V or I; where the T42-2 polypeptide has a length of 42 amino acids.

In some cases, a T42-2 polypeptide comprises the amino acid sequence KGGRHLIFCHSKKKCDELAAKLRGMGLNAVAYYRGLDVSVIP (SEQ ID NO:151); and has a length of 42 amino acids.

### TP65 Polypeptides

In some cases, a TP65 T-cell epitope polypeptide (a “TP65 polypeptide”) comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: TPIDTTIMAKNEVFCVDPX₆KGGRKPARLIVYPDLGVRVCEKMALYDVVQKLPQAVMGX₇SYGF QYS (SEQ ID NO:152), where X₆ is E, T, or V, and X₇ is S, A, or P; and has a length of from 65 amino acids to 70 amino acids (e.g., 65, 66, 67, 68, 69, or 70 amino acids). In some cases, a TP65 T-cell epitope polypeptide (a “TP65 polypeptide”) comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: TPIDTTIMAKNEVFCVDPEKGGRKPARLIVYPDLGVRVCEKMALYDVVQKLPQAVMGSSYGFQ YS (SEQ ID NO:153) and has a length of 65 amino acids. In some cases, the TP has a length of 65 amino acids. In some cases, a TP65 polypeptide has the following amino acid sequence: TPIDTTIMAKNEVFCVDPEKGGRKPARLIVYPDLGVRVCEKMALYDVVQKLPQAVMGSSYGFQ YS (SEQ ID NO:153) and has a length of 65 amino acids. In some cases, a TP65 polypeptide has the following amino acid sequence: TPIDTTIMAKNEVFCVDPTKGGRKPARLIVYPDLGVRVCEKMALYDVVQKLPQAVMGASYGFQ YS (SEQ ID NO:154) and has a length of 65 amino acids. In some cases, a TP65 polypeptide has the following amino acid sequence:

(SEQ ID NO: 155)
TPIDTTIMAKNEVFCVDPVKGGRKPARLIVYPDLGVRVCEKMALYDVVQ
KLPQAVMGPSYGFQYS.

### TP240 Polypeptides

In some cases, a TP240 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence:

(SEQ ID NO: 156)
SYQVGYLHAPTGSGKSTKVPAAYAAQGYKVLVLNPSVAATLGFGAYMSK
AHGIDPNIRTGVRTVTTGAPITYSTYGKFLADGGCSGGAYDIIICDECH
SX₁DATTILGIGTVLDQAETAGARLVVLATATPPGSVTVPHPNIEEVAL
GX₂EGEIPFYGKAIPLX₃X₄IKGGRHLIFCHSKKKCDELAAKLRGMGLN
AVAYYRGLDVSVIPTX₅GDVVVVATDALMTGYTGDFDSVIDCNVAVTQT,

where X₁ is T, V, or Q, X₂ is T, N, Q, H, or S, X₃ is E, S, or A, X₄ is V, T, Y, F, or L, and X₅ is S, Q, or T; and has a length of from 240 amino acids to 245 amino acids (e.g., 240, 241, 242, 243, 244, or 245 amino acids). In some cases, the TP has a length of 240 amino acids.

In some cases, a TP240 polypeptide has the following amino acid sequence: SYQVGYLHAPTGSGKSTKVPAAYAAQGYKVLVLNPSVAATLGFGAYMSKAHGIDPNIRTGVRT VTTGAPITYSTYGKFLADGGCSGGAYDIIICDECHSTDATTILGIGTVLDQAETAGARLVVLATA TPPGSVTVPHPNIEEVALGTEGEIPFYGKAIPLEVIKGGRHLIFCHSKKKCDELAAKLRGMGLNA VAYYRGLDVSVIPTSGDVVVVATDALMTGYTGDFDSVIDCNVAVTQT (SEQ ID NO:157), and has a length of 240 amino acids. In some cases, a TP240 polypeptide has the following amino acid sequence: SYQVGYLHAPTGSGKSTKVPAAYAAQGYKVLVLNPSVAATLGFGAYMSKAHGIDPNIRTGVRT VTTGAPITYSTYGKFLADGGCSGGAYDIIICDECHSVDATTILGIGTVLDQAETAGARLVVLATA TPPGSVTVPHPNIEEVALGNEGEIPFYGKAIPLSTIKGGRHLIFCHSKKKCDELAAKLRGMGLNA VAYYRGLDVSVIPTQGDVVVVATDALMTGYTGDFDSVIDCNVAVTQT (SEQ ID NO:158), and has a length of 240 amino acids.

### TP465 Polypeptides

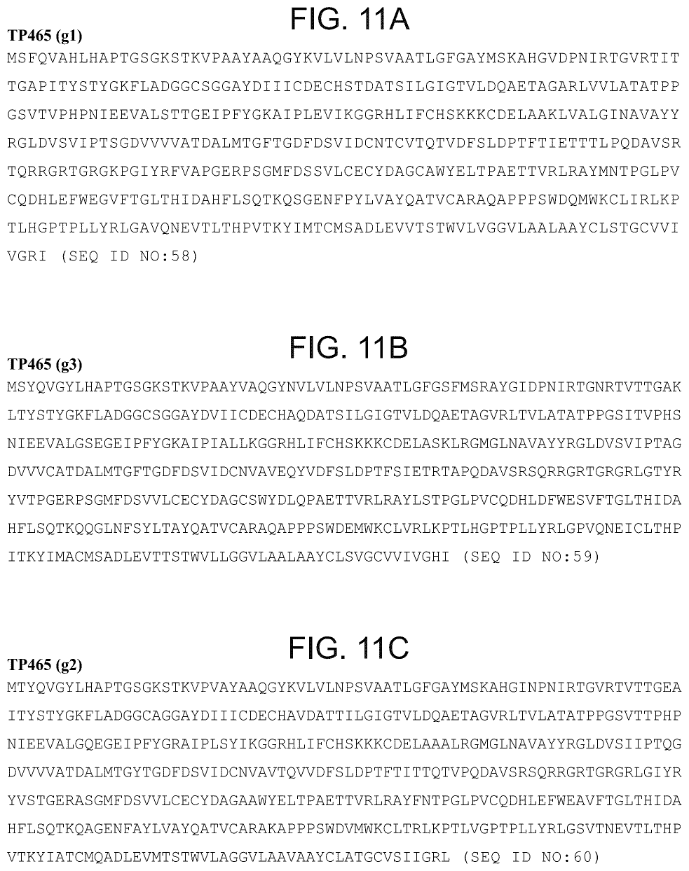

In some cases, a TP465 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to any one of the TP465 polypeptide amino acid sequences depicted in FIG. 11A-11C; and has a length of from 460 amino acids to 470 amino acids, e.g., has a length of 465 amino acids.

In some cases, a TP465 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence:

SFQVAHLHAPTGSGKSTKVPAAYAAQGYKVLVLNPSVAATLGFGAYMSKAH GVDPNIRTGVRTITTGAPITYSTYGKFLADGGCSGGAYDIIICDECHSTDATSILGIGTVLDQ AETAGARLVVLATATPPGSVTVPHPNIEEVALSTTGEIPFYGKAIPLEVIKGGRHLIFCHSKK KCDELAAKLVALGINAVAYYRGLDVSVIPTSGDVVVVATDALMTGFTGDFDSVIDCNTCVT QTVDFSLDPTFTIETTTLPQDAVSRTQRRGRTGRGKPGIYRFVAPGERPSGMFDSSVLCECY DAGCAWYELTPAETTVRLRAYMNTPGLPVCQDHLEFWEGVFTGLTHIDAHFLSQTKQSGE NFPYLVAYQATVCARAQAPPPSWDQMWKCLIRLKPTLHGPTPLLYRLGAVQNEVTLTHPVT KYIMTCMSADLEVVTSTWVLVGGVLAALAAYCLSTGCVVIVGRI (SEQ ID NO:159); and has a length of about 465 amino acids.

### TP156 Polypeptides

In some cases, a TP156 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence:

KFPGGGQIVGGVYLLPRRGPRLGVRATRKTSERSQPRGRRQPIPKARRPEGRTW AQPGYPWPLYGNEGCGWAGWLLSPRGSRPSWGPTDPRRRSRNLGKVIDTLTCGFADLMGYIPL VGAPLGGAARALAHGVRVLEDGVNYATGNLPGCSFSIFL (SEQ ID NO:160); and has a length of from about 150 amino acids to about 169 amino acids; e.g., has a length of 156 amino acids.

### TP29 Polypeptides

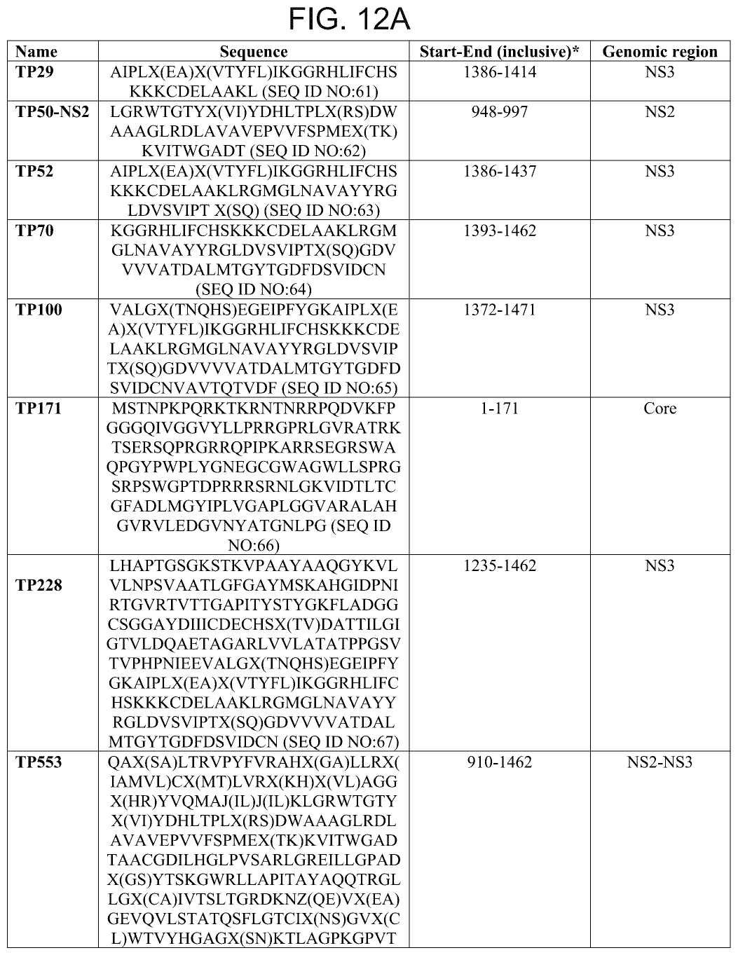

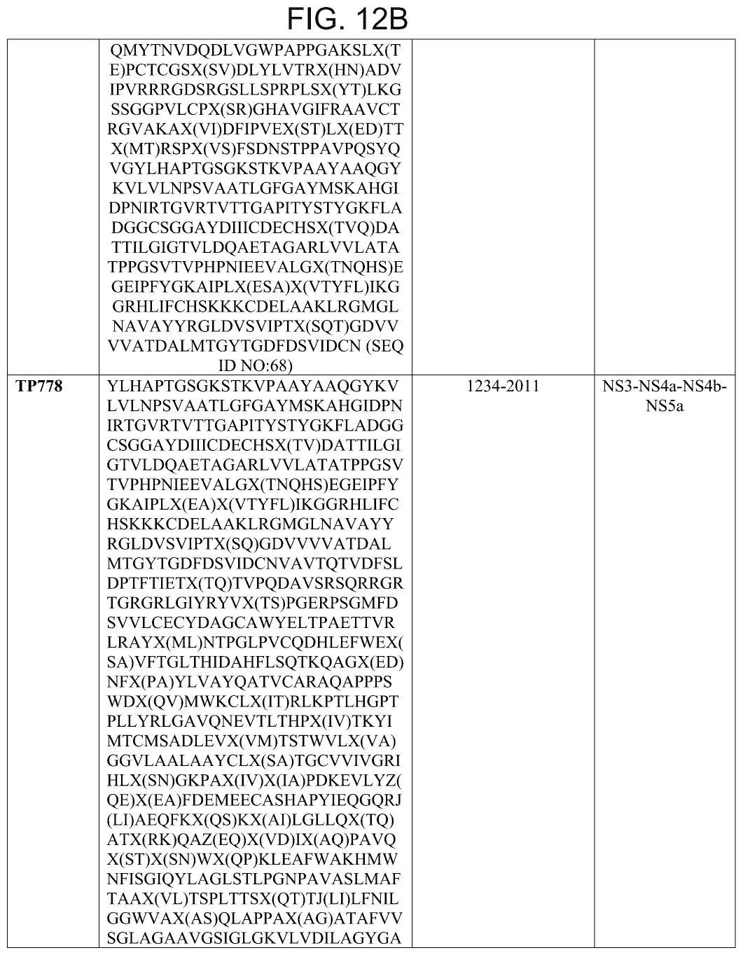

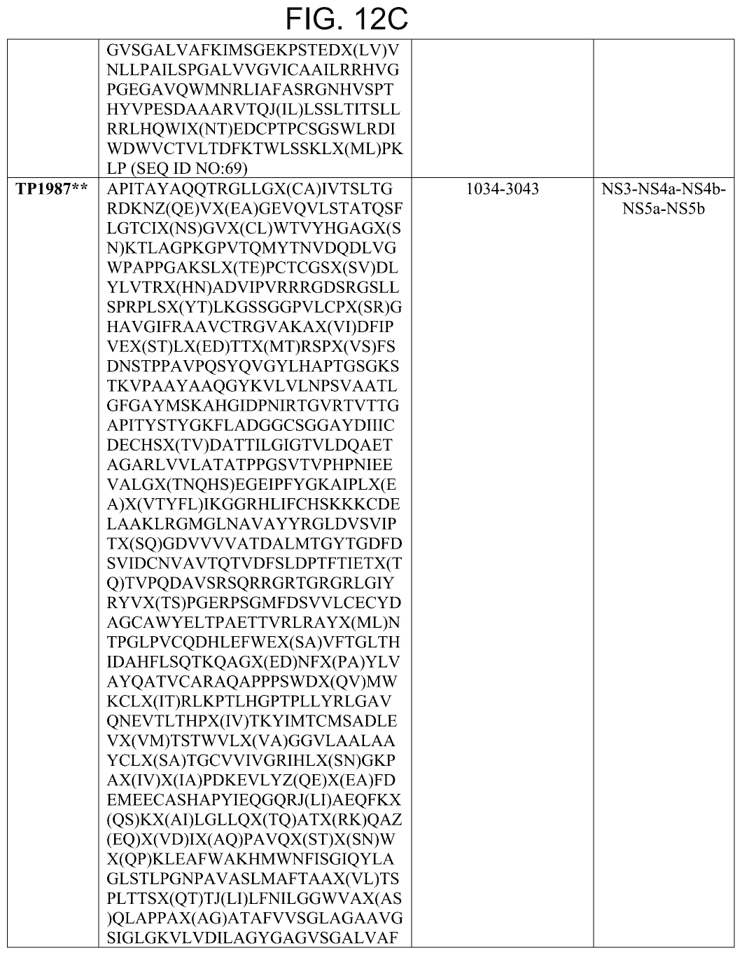

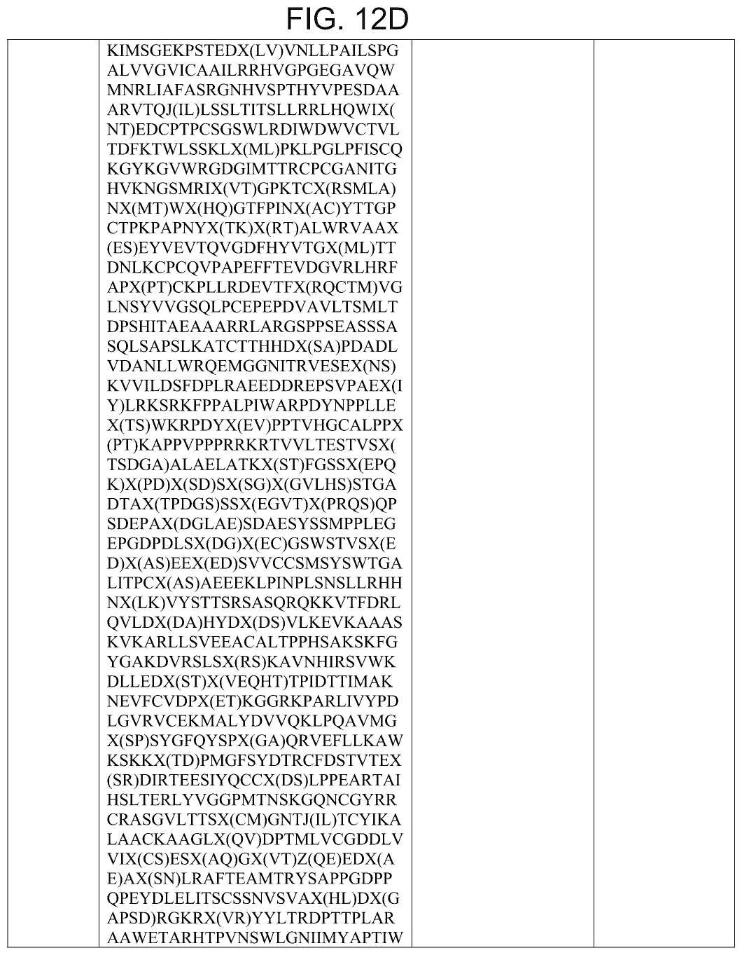

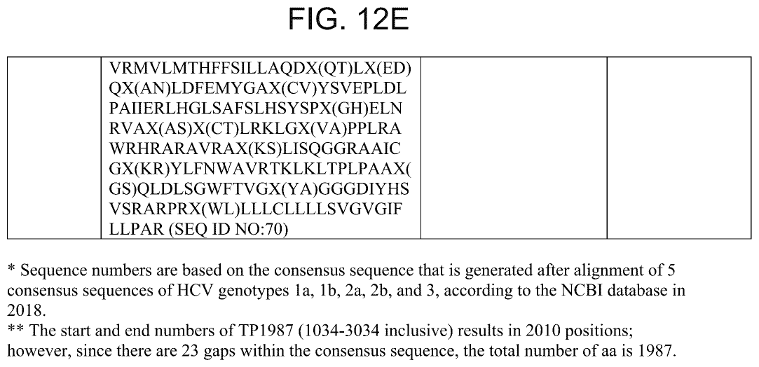

In some cases, a TP29 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP29 amino acid sequence depicted in FIG. 12A-12E; and has a length of about 29 amino acids. For example, in some cases, a TP29 polypeptide comprises the amino acid sequence: AIPLX(EA)X(VTYFL)IKGGRHLIFCHSKKKCDELAAKL (SEQ ID NO:61), where the first “X” is Glu or Ala, and the second “X” is Val, Thr, Tyr, Phe, or Leu; and where the TP29 polypeptide has a length of 29 amino acids.

### TP50-NS2 Polypeptides

In some cases, a TP50-NS2 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP50-NS2 amino acid sequence depicted in FIG. 12A-12E; and has a length of about 50 amino acids. For example, in some cases, a TP50-NS2 polypeptide comprises the amino acid sequence: LGRWTGTYX(VI)YDHLTPLX(RS)DWAAAGLRDLAVAVEPVVFSPMEX(TK)KVITWGADT (SEQ ID NO:62), where the first “X” is Val or Ile, the second “X” is Arg or Ser, and the third “X” is Thr or Lys; and where the TP50-NS2 polypeptide has a length of 50 amino acids.

### TP52 Polypeptides

In some cases, a TP52 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP52 amino acid sequence depicted in FIG. 12A-12E; and has a length of about 52 amino acids. For example, in some cases, a TP52 polypeptide comprises the amino acid sequence: AIPLX(EA)X(VTYFL)IKGGRHLIFCHSKKKCDELAAKLRGMGLNAVAYYRGLDVSVIPT X(SQ) (SEQ ID NO:63), where the first “X” is Glu or Ala, the second “X” is Val, Thr, Tyr, Phe, or Leu, and the third “X” is Ser or Gln; and where the TP52 polypeptide has a length of 52 amino acids.

### TP70 Polypeptides

In some cases, a TP70 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP70 amino acid sequence depicted in FIG. 12A-12E; and has a length of about 70 amino acids. For example, in some cases, a TP70 polypeptide comprises the TP70 amino acid sequence depicted in FIG. 12A-12E, where X is Ser or Gln; and where the TP70 polypeptide has a length of 70 amino acids.

### TP100 Polypeptides

In some cases, a TP100 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP100 amino acid sequence depicted in FIG. 12A-12E; and has a length of about 100 amino acids. For example, in some cases, a TP100 polypeptide comprises the TP100 amino acid sequence depicted in FIG. 12A-12E, where the first “X” is Thr, Asn, Gln, His, or Ser, where the second “X” is Glue or Ala, the third “X” is Val, Thr, Tyr, Phe, or Leu, and the fourth “X” is Ser or Gln; and where the TP100 polypeptide has a length of 100 amino acids.

### TP171 Polypeptides

In some cases, a TP171 comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP171 amino acid sequence depicted in FIG. 12A-12E; and has a length of about 171 amino acids.

### TP228 Polypeptides

In some cases, a TP228 comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP228 amino acid sequence depicted in FIG. 12A-12E; and has a length of about 228 amino acids.

A TP228 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following TP228 amino acid sequence: LHAPTGSGKSTKVPAAYAAQGYKVLVLNPSVAATLGFGAYMSKAHGIDPNIRTGVRTVTTGAP ITYSTYGKFLADGGCSGGAYDIIICDECHSXIDATTILGIGTVLDQAETAGARLVVLATATPPGSV TVPHPNIEEVALGX₂EGEIPFYGKAIPLX₃X₄IKGGRHLIFCHSKKKCDELAAKLRGMGLNAVAYY RGLDVSVIPTXsGDVVVVATDALMTGYTGDFDSVIDCN (SEQ ID NO:161), where X₁ is Thr or Val; X₂ is Thr, Asn, Gln, His, or Ser; X₃ is Glu or Ala; X₄ is Val, Thr, Tyr, Phe, or Leu; and X₅ is Ser or Gln; and can have a length of 228 amino acids.

For example, a TP228 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LHAPTGSGKSTKVPAAYAAQGYKVLVLNPSVAATLGFGAYMSKAHGIDPNIRTGVRTITTGSPI TYSTYGKFLADGGCSGGAYDIIICDECHSTDATSILGIGTVLDQAETAGARLVVLATATPPGSVT VPHPNIEEVALSTTGEIPFYGKAIPLEVIKGGRHLIFCHSKKKCDELAAKLVALGINAVAYYRGL DVSVIPTSGDVVVVATDALMTGFTGDFDSVIDCN (SEQ ID NO:162); and can have a length of from 215 amino acids (aa) to 235 aa (e.g., 215 aa, 216 aa, 217 11, 218 aa, 219 aa, 220 aa, 221 aa, 222 aa, 223 aa, 224 aa, 225 aa, 226 aa, 227 aa, 228 aa, 229 aa, 230 aa, 231 aa, 232 aa, 233 aa, 234 aa, or 235 aa). In some cases, a TP228 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LHAPTGSGKSTKVPAAYAAQGYKVLVLNPSVAATLGFGAYMSKAHGIDPNIRTGVRTITTGSPI TYSTYGKFLADGGCSGGAYDIIICDECHSTDATSILGIGTVLDQAETAGARLVVLATATPPGSVT VPHPNIEEVALSTTGEIPFYGKAIPLEVIKGGRHLIFCHSKKKCDELAAKLVALGINAVAYYRGL DVSVIPTSGDVVVVATDALMTGFTGDFDSVIDCN (SEQ ID NO:163); and has a length of 228 amino acids.

### TP553 Polypeptides

In some cases, a TP553 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP553 amino acid sequence depicted in FIG. 12A-12E; and has a length of about 553 amino acids.

### TP778 Polypeptides

In some cases, a TP778 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP778 amino acid sequence depicted in FIG. 12A-12E; and has a length of about 778 amino acids.

### TP1987 Polypeptides

In some cases, a TP1987 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP1987 amino acid sequence depicted in FIG. 12A-12E; and has a length of about 1987 amino acids.

### Compositions Comprising a Single TP

In some cases, an immunogenic composition of the present disclosure comprises: a) a single TP, where the TP is selected from the group consisting of: i) a TP35-NS3 polypeptide; ii) a TP50C polypeptide; iii) a TP23 polypeptide; iv) a TP27 polypeptide; v) a TP35-NS4 polypeptide; vi) a TP42 polypeptide; vii) a TP45 polypeptide; viii) a TP48 polypeptide; ix) a TP33 polypeptide; x) a TP42-2 polypeptide; xi) a TP65 polypeptide; xii) a TP240 polypeptide; xiii) a TP156 polypeptide; xiv) a TP465 polypeptide; xv) a TP29 polypeptide; xvi) a TP50-NS2 polypeptide; xvii) a TP52 polypeptide; xviii) a TP70 polypeptide; xix) a TP100 polypeptide; xx) a TP171 polypeptide; xxi) TP228 polypeptide; xxii) a TP553 polypeptide; xxiii) a TP778 polypeptide; and xxiv) a TP1987 polypeptide; and b) SLA formulated in: i) a stable emulsion; or ii) a liposomal composition comprising a saponin. In some cases, an immunogenic composition of the present disclosure comprises: a) a single TP, where the TP is selected from the group consisting of: i) a TP35-NS3 polypeptide; ii) a TP50C polypeptide; iii) a TP23 polypeptide; iv) a TP27 polypeptide; v) a TP35-NS4 polypeptide; vi) a TP42 polypeptide; vii) a TP45 polypeptide; viii) a TP48 polypeptide; ix) a TP33 polypeptide; x) a TP42-2 polypeptide; xi) a TP65 polypeptide; xii) a TP240 polypeptide; xiii) a TP156 polypeptide; xiv) a TP465 polypeptide; xv) a TP29 polypeptide; xvi) a TP50-NS2 polypeptide; xvii) a TP52 polypeptide; xviii) a TP70 polypeptide; xix) a TP100 polypeptide; xx) a TP171 polypeptide; xxi) TP228 polypeptide; xxii) a TP553 polypeptide; xxiii) a TP778 polypeptide; and xxiv) a TP1987 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) a single TP, where the TP is selected from the group consisting of: a TP35-NS3 polypeptide, a TP50C polypeptide, a TP27 polypeptide, a TP48 polypeptide, a TP65 polypeptide, and a TP240 polypeptide; and b) SLA formulated in: i) a stable emulsion; or ii) a liposomal composition comprising a saponin. In some cases, an immunogenic composition of the present disclosure comprises: a) a single TP, where the TP is selected from the group consisting of: a TP35-NS3 polypeptide, a TP50C polypeptide, a TP27 polypeptide, a TP48 polypeptide, a TP65 polypeptide, and a TP240 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) a single TP, where the TP is selected from the group consisting of: a TP35-NS3 polypeptide, a TP50C polypeptide, a TP27 polypeptide, a TP48 polypeptide, a TP65 polypeptide, a TP240 polypeptide, a TP156 polypeptide, and a TP465 polypeptide; and b) SLA formulated in: i) a stable emulsion; or ii) a liposomal composition comprising a saponin. In some cases, an immunogenic composition of the present disclosure comprises: a) a single TP, where the TP is selected from the group consisting of: a TP35-NS3 polypeptide, a TP50C polypeptide, a TP27 polypeptide, a TP48 polypeptide, a TP65 polypeptide, a TP240 polypeptide, a TP156 polypeptide, and a TP465 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) a single TP, where the TP is a TP35-NS3 polypeptide; and b) SLA formulated in: i) a stable emulsion; or ii) a liposomal composition comprising a saponin. In some cases, an immunogenic composition of the present disclosure comprises: a) a single TP, where the TP is a TP35-NS3 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) a single TP, where the TP is a TP27 polypeptide; and b) SLA formulated in: i) a stable emulsion; or ii) a liposomal composition comprising a saponin. In some cases, an immunogenic composition of the present disclosure comprises: a) a single TP, where the TP is a TP27 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) a single TP, where the TP is a TP48 polypeptide; and b) SLA formulated in: i) a stable emulsion; or ii) a liposomal composition comprising a saponin. In some cases, an immunogenic composition of the present disclosure comprises: a) a single TP, where the TP is a TP48 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) a single TP, where the TP is a TP50C polypeptide; and b) SLA formulated in: i) a stable emulsion; or ii) a liposomal composition comprising a saponin. In some cases, an immunogenic composition of the present disclosure comprises: a) a single TP, where the TP is a TP50C polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) a single TP, where the TP is a TP65 polypeptide; and b) SLA formulated in: i) a stable emulsion; or ii) a liposomal composition comprising a saponin. In some cases, an immunogenic composition of the present disclosure comprises: a) a single TP, where the TP is a TP65 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) a single TP, where the TP is a TP240 polypeptide; and b) SLA formulated in: i) a stable emulsion; or ii) a liposomal composition comprising a saponin. In some cases, an immunogenic composition of the present disclosure comprises: a) a single TP, where the TP is a TP240 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) a single TP, where the TP is a TP156 polypeptide; and b) SLA formulated in: i) a stable emulsion; or ii) a liposomal composition comprising a saponin. In some cases, an immunogenic composition of the present disclosure comprises: a) a single TP, where the TP is a TP156 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) a single TP, where the TP is a TP465 polypeptide; and b) SLA formulated in: i) a stable emulsion; or ii) a liposomal composition comprising a saponin. In some cases, an immunogenic composition of the present disclosure comprises: a) a single TP, where the TP is a TP465 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

### Compositions Comprising Two TPs

In some cases, an immunogenic composition of the present disclosure comprises: a) only two TPs, where the two TPs are selected from the group consisting of: i) a TP35-NS3 polypeptide; ii) a TP50C polypeptide; iii) a TP23 polypeptide; iv) a TP27 polypeptide; v) a TP35-NS4 polypeptide; vi) a TP42 polypeptide; vii) a TP45 polypeptide; viii) a TP48 polypeptide; ix) a TP33 polypeptide; x) a TP42-2 polypeptide; xi) a TP65 polypeptide; xii) a TP240 polypeptide; xiii) a TP156 polypeptide; xiv) a TP465 polypeptide; xv) a TP29 polypeptide; xvi) a TP50-NS2 polypeptide; xvii) a TP52 polypeptide; xviii) a TP70 polypeptide; xix) a TP100 polypeptide; xx) a TP171 polypeptide; xxi) TP228 polypeptide; xxii) a TP553 polypeptide; xxiii) a TP778 polypeptide; and xxiv) a TP1987 polypeptide; and b) SLA formulated in: i) a stable emulsion; or ii) a liposomal composition comprising a saponin. In some cases, an immunogenic composition of the present disclosure comprises: a) only two TPs, where the two TPs are selected from the group consisting of: i) a TP35-NS3 polypeptide; ii) a TP50C polypeptide; iii) a TP23 polypeptide; iv) a TP27 polypeptide; v) a TP35-NS4 polypeptide; vi) a TP42 polypeptide; vii) a TP45 polypeptide; viii) a TP48 polypeptide; ix) a TP33 polypeptide; x) a TP42-2 polypeptide; xi) a TP65 polypeptide; xii) a TP240 polypeptide; xiii) a TP156 polypeptide; xiv) a TP465 polypeptide; xv) a TP29 polypeptide; xvi) a TP50-NS2 polypeptide; xvii) a TP52 polypeptide; xviii) a TP70 polypeptide; xix) a TP100 polypeptide; xx) a TP171 polypeptide; xxi) TP228 polypeptide; xxii) a TP553 polypeptide; xxiii) a TP778 polypeptide; and xxiv) a TP1987 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) only two TPs, where the two TPs are selected from the group consisting of: a TP35-NS3 polypeptide, a TP50C polypeptide, a TP27 polypeptide, a TP48 polypeptide, a TP65 polypeptide, and a TP240 polypeptide; and b) SLA formulated in: i) a stable emulsion; or ii) a liposomal composition comprising a saponin. In some cases, an immunogenic composition of the present disclosure comprises: a) only two TPs, where the two TPs are selected from the group consisting of: a TP35-NS3 polypeptide, a TP50C polypeptide, a TP27 polypeptide, a TP48 polypeptide, a TP65 polypeptide, and a TP240 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) only two TPs, where the two TPs are selected from the group consisting of: a TP35-NS3 polypeptide, a TP50C polypeptide, a TP27 polypeptide, a TP48 polypeptide, a TP65 polypeptide, a TP240 polypeptide, a TP156 polypeptide, and a TP465 polypeptide; and b) SLA formulated in: i) a stable emulsion; or ii) a liposomal composition comprising a saponin. In some cases, an immunogenic composition of the present disclosure comprises: a) only two TPs, where the two TPs are selected from the group consisting of: a TP35-NS3 polypeptide, a TP50C polypeptide, a TP27 polypeptide, a TP48 polypeptide, a TP65 polypeptide, a TP240 polypeptide, a TP156 polypeptide, and a TP465 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) two TPs, where the two TPs are a TP35-NS3 polypeptide and a TP27 polypeptide; and b) SLA formulated in: i) a stable emulsion; or ii) a liposomal composition comprising a saponin. In some cases, an immunogenic composition of the present disclosure comprises: a) two TPs, where the two TPs are a TP35-NS3 polypeptide and a TP27 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) two TPs, where the two TPs are a TP48 polypeptide and a TP27 polypeptide; and b) SLA formulated in: i) a stable emulsion; or ii) a liposomal composition comprising a saponin. In some cases, an immunogenic composition of the present disclosure comprises: a) two TPs, where the two TPs are a TP48 polypeptide and a TP27 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) two TPs, where the two TPs are a TP27 polypeptide and a TP50C polypeptide; and b) SLA formulated in: i) a stable emulsion; or ii) a liposomal composition comprising a saponin. In some cases, an immunogenic composition of the present disclosure comprises: a) two TPs, where the two TPs are a TP27 polypeptide and a TP50C polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) two TPs, where the two TPs are a TP35-NS3 polypeptide and a TP48 polypeptide; and b) SLA formulated in: i) a stable emulsion; or ii) a liposomal composition comprising a saponin. In some cases, an immunogenic composition of the present disclosure comprises: a) two TPs, where the two TPs are a TP35-NS3 polypeptide and a TP48 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) two TPs, where the two TPs are a TP35-NS3 polypeptide and a TP50C polypeptide; and b) SLA formulated in: i) a stable emulsion; or ii) a liposomal composition comprising a saponin. In some cases, an immunogenic composition of the present disclosure comprises: a) two TPs, where the two TPs are a TP35-NS3 polypeptide and a TP50C polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) two TPs, where the two TPs are a TP48 polypeptide and a TP50C polypeptide; and b) SLA formulated in: i) a stable emulsion; or ii) a liposomal composition comprising a saponin. In some cases, an immunogenic composition of the present disclosure comprises: a) two TPs, where the two TPs are a TP48 polypeptide and a TP50C polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) two TPs, where the two TPs are a TP156 polypeptide and a TP465 polypeptide; and b) SLA formulated in: i) a stable emulsion; or ii) a liposomal composition comprising a saponin. In some cases, an immunogenic composition of the present disclosure comprises: a) two TPs, where the two TPs are a TP156 polypeptide and a TP465 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) two TPs, where the two TPs are a TP156 polypeptide and a TP48 polypeptide; and b) SLA formulated in: i) a stable emulsion; or ii) a liposomal composition comprising a saponin. In some cases, an immunogenic composition of the present disclosure comprises: a) two TPs, where the two TPs are a TP156 polypeptide and a TP48 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) two TPs, where the two TPs are a TP48 polypeptide and a TP465 polypeptide; and b) SLA formulated in: i) a stable emulsion; or ii) a liposomal composition comprising a saponin. In some cases, an immunogenic composition of the present disclosure comprises: a) two TPs, where the two TPs are a TP48 polypeptide and a TP465 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) two TPs, where the two TPs are a TP48 polypeptide and a TP65 polypeptide; and b) SLA formulated in: i) a stable emulsion; or ii) a liposomal composition comprising a saponin. In some cases, an immunogenic composition of the present disclosure comprises: a) two TPs, where the two TPs are a TP48 polypeptide and a TP65 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) two TPs, where the two TPs are a TP465 polypeptide and a TP65 polypeptide; and b) SLA formulated in: i) a stable emulsion; or ii) a liposomal composition comprising a saponin. In some cases, an immunogenic composition of the present disclosure comprises: a) two TPs, where the two TPs are a TP465 polypeptide and a TP65 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

### Compositions Comprising Three TPs

In some cases, an immunogenic composition of the present disclosure comprises: a) only three TPs, where the three TPs are selected from the group consisting of: i) a TP35-NS3 polypeptide; ii) a TP50C polypeptide; iii) a TP23 polypeptide; iv) a TP27 polypeptide; v) a TP35-NS4 polypeptide; vi) a TP42 polypeptide; vii) a TP45 polypeptide; viii) a TP48 polypeptide; ix) a TP33 polypeptide; x) a TP42-2 polypeptide; xi) a TP65 polypeptide; xii) a TP240 polypeptide; xiii) a TP156 polypeptide; xiv) a TP465 polypeptide; xv) a TP29 polypeptide; xvi) a TP50-NS2 polypeptide; xvii) a TP52 polypeptide; xviii) a TP70 polypeptide; xix) a TP100 polypeptide; xx) a TP171 polypeptide; xxi) TP228 polypeptide; xxii) a TP553 polypeptide; xxiii) a TP778 polypeptide; and xxiv) a TP1987 polypeptide; and b) SLA formulated in: i) a stable emulsion; or ii) a liposomal composition comprising a saponin. In some cases, an immunogenic composition of the present disclosure comprises: a) only three TPs, where the three TPs are selected from the group consisting of: i) a TP35-NS3 polypeptide; ii) a TP50C polypeptide; iii) a TP23 polypeptide; iv) a TP27 polypeptide; v) a TP35-NS4 polypeptide; vi) a TP42 polypeptide; vii) a TP45 polypeptide; viii) a TP48 polypeptide; ix) a TP33 polypeptide; x) a TP42-2 polypeptide; xi) a TP65 polypeptide; xii) a TP240 polypeptide; xiii) a TP156 polypeptide; xiv) a TP465 polypeptide; xv) a TP29 polypeptide; xvi) a TP50-NS2 polypeptide; xvii) a TP52 polypeptide; xviii) a TP70 polypeptide; xix) a TP100 polypeptide; xx) a TP171 polypeptide; xxi) TP228 polypeptide; xxii) a TP553 polypeptide; xxiii) a TP778 polypeptide; and xxiv) a TP1987 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) only three TPs, where the three TPs are selected from the group consisting of: a TP35-NS3 polypeptide, a TP50C polypeptide, a TP27 polypeptide, a TP48 polypeptide, a TP65 polypeptide, and a TP240 polypeptide; and b) SLA formulated in: i) a stable emulsion; or ii) a liposomal composition comprising a saponin. In some cases, an immunogenic composition of the present disclosure comprises: a) only three TPs, where the three TPs are selected from the group consisting of: a TP35-NS3 polypeptide, a TP50C polypeptide, a TP27 polypeptide, a TP48 polypeptide, a TP65 polypeptide, and a TP240 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) only three TPs, where the three TPs are selected from the group consisting of: a TP35-NS3 polypeptide, a TP50C polypeptide, a TP27 polypeptide, a TP48 polypeptide, a TP65 polypeptide, a TP240 polypeptide, a TP156 polypeptide, and a TP465 polypeptide; and b) SLA formulated in: i) a stable emulsion; or ii) a liposomal composition comprising a saponin. In some cases, an immunogenic composition of the present disclosure comprises: a) only three TPs, where the three TPs are selected from the group consisting of: a TP35-NS3 polypeptide, a TP50C polypeptide, a TP27 polypeptide, a TP48 polypeptide, a TP65 polypeptide, a TP240 polypeptide, a TP156 polypeptide, and a TP465 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) three TPs, where the three TPs are a TP35-NS3 polypeptide, a TP48 polypeptide, and a TP27 polypeptide; and b) SLA formulated in: i) a stable emulsion; or ii) a liposomal composition comprising a saponin. In some cases, an immunogenic composition of the present disclosure comprises: a) three TPs, where the three TPs are a TP35-NS3 polypeptide, a TP48 polypeptide, and a TP27 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) three TPs, where the three TPs are a TP50C polypeptide, a TP48 polypeptide, and a TP27 polypeptide; and b) SLA formulated in: i) a stable emulsion; or ii) a liposomal composition comprising a saponin. In some cases, an immunogenic composition of the present disclosure comprises: a) three TPs, where the three TPs are a TP50C polypeptide, a TP48 polypeptide, and a TP27 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) three TPs, where the three TPs are a TP50C polypeptide, a TP48 polypeptide, and a TP35-NS3 polypeptide; and b) SLA formulated in: i) a stable emulsion; or ii) a liposomal composition comprising a saponin. In some cases, an immunogenic composition of the present disclosure comprises: a) three TPs, where the three TPs are a TP50C polypeptide, a TP48 polypeptide, and a TP35-NS3 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) three TPs, where the three TPs are a TP50C polypeptide, a TP27 polypeptide, and a TP35-NS3 polypeptide; and b) SLA formulated in: i) a stable emulsion; or ii) a liposomal composition comprising a saponin. In some cases, an immunogenic composition of the present disclosure comprises: a) three TPs, where the three TPs are a TP50C polypeptide, a TP27 polypeptide, and a TP35-NS3 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) three TPs, where the three TPs are a TP156 polypeptide, a TP465 polypeptide, and a TP48 polypeptide; and b) SLA formulated in: i) a stable emulsion; or ii) a liposomal composition comprising a saponin. In some cases, an immunogenic composition of the present disclosure comprises: a) three TPs, where the three TPs are a TP156 polypeptide, a TP465 polypeptide, and a TP48 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) three TPs, where the three TPs are a TP156 polypeptide, a TP465 polypeptide, and a TP65 polypeptide; and b) SLA formulated in: i) a stable emulsion; or ii) a liposomal composition comprising a saponin. In some cases, an immunogenic composition of the present disclosure comprises: a) three TPs, where the three TPs are a TP156 polypeptide, a TP465 polypeptide, and a TP65 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) three TPs, where the three TPs are a TP465 polypeptide, a TP48 polypeptide, and a TP65 polypeptide; and b) SLA formulated in: i) a stable emulsion; or ii) a liposomal composition comprising a saponin. In some cases, an immunogenic composition of the present disclosure comprises: a) three TPs, where the three TPs are a TP465 polypeptide, a TP48 polypeptide, and a TP65 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) three TPs, where the three TPs are a TP156 polypeptide, a TP48 polypeptide, and a TP65 polypeptide; and b) SLA formulated in: i) a stable emulsion; or ii) a liposomal composition comprising a saponin. In some cases, an immunogenic composition of the present disclosure comprises: a) three TPs, where the three TPs are a TP156 polypeptide, a TP48 polypeptide, and a TP65 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

### Compositions Comprising Four TPs

In some cases, an immunogenic composition of the present disclosure comprises: a) only four TPs, where the four TPs are selected from the group consisting of: i) a TP35-NS3 polypeptide; ii) a TP50C polypeptide; iii) a TP23 polypeptide; iv) a TP27 polypeptide; v) a TP35-NS4 polypeptide; vi) a TP42 polypeptide; vii) a TP45 polypeptide; viii) a TP48 polypeptide; ix) a TP33 polypeptide; x) a TP42-2 polypeptide; xi) a TP65 polypeptide; xii) a TP240 polypeptide; xiii) a TP156 polypeptide; xiv) a TP465 polypeptide; xv) a TP29 polypeptide; xvi) a TP50-NS2 polypeptide; xvii) a TP52 polypeptide; xviii) a TP70 polypeptide; xix) a TP100 polypeptide; xx) a TP171 polypeptide; xxi) TP228 polypeptide; xxii) a TP553 polypeptide; xxiii) a TP778 polypeptide; and xxiv) a TP1987 polypeptide; and b) SLA formulated in: i) a stable emulsion; or ii) a liposomal composition comprising a saponin. In some cases, an immunogenic composition of the present disclosure comprises: a) only four TPs, where the four TPs are selected from the group consisting of: i) a TP35-NS3 polypeptide; ii) a TP50C polypeptide; iii) a TP23 polypeptide; iv) a TP27 polypeptide; v) a TP35-NS4 polypeptide; vi) a TP42 polypeptide; vii) a TP45 polypeptide; viii) a TP48 polypeptide; ix) a TP33 polypeptide; x) a TP42-2 polypeptide; xi) a TP65 polypeptide; xii) a TP240 polypeptide; xiii) a TP156 polypeptide; xiv) a TP465 polypeptide; xv) a TP29 polypeptide; xvi) a TP50-NS2 polypeptide; xvii) a TP52 polypeptide; xviii) a TP70 polypeptide; xix) a TP100 polypeptide; xx) a TP171 polypeptide; xxi) TP228 polypeptide; xxii) a TP553 polypeptide; xxiii) a TP778 polypeptide; and xxiv) a TP1987 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) only four TPs, where the four TPs are selected from the group consisting of: a TP35-NS3 polypeptide, a TP50C polypeptide, a TP27 polypeptide, a TP48 polypeptide, a TP65 polypeptide, and a TP240 polypeptide; and b) SLA formulated in: i) a stable emulsion; or ii) a liposomal composition comprising a saponin. In some cases, an immunogenic composition of the present disclosure comprises: a) only four TPs, where the four TPs are selected from the group consisting of: a TP35-NS3 polypeptide, a TP50C polypeptide, a TP27 polypeptide, a TP48 polypeptide, a TP65 polypeptide, and a TP240 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) only four TPs, where the four TPs are selected from the group consisting of: a TP35-NS3 polypeptide, a TP50C polypeptide, a TP27 polypeptide, a TP48 polypeptide, a TP65 polypeptide, a TP240 polypeptide, a TP156 polypeptide, and a TP465 polypeptide; and b) SLA formulated in: i) a stable emulsion; or ii) a liposomal composition comprising a saponin. In some cases, an immunogenic composition of the present disclosure comprises: a) only four TPs, where the four TPs are selected from the group consisting of: a TP35-NS3 polypeptide, a TP50C polypeptide, a TP27 polypeptide, a TP48 polypeptide, a TP65 polypeptide, a TP240 polypeptide, a TP156 polypeptide, and a TP465 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) four TPs, where the four TPs are a TP35-NS3 polypeptide, a TP48 polypeptide, a TP50C polypeptide, and a TP27 polypeptide; and b) SLA formulated in: i) a stable emulsion; or ii) a liposomal composition comprising a saponin. In some cases, an immunogenic composition of the present disclosure comprises: a) four TPs, where the four TPs are a TP35-NS3 polypeptide, a TP48 polypeptide, a TP50C polypeptide, and a TP27 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) four TPs, where the four TPs are a TP240 polypeptide, a TP48 polypeptide, a TP65 polypeptide, and a TP50C polypeptide; and b) SLA formulated in: i) a stable emulsion; or ii) a liposomal composition comprising a saponin. In some cases, an immunogenic composition of the present disclosure comprises: a) four TPs, where the four TPs are a TP240 polypeptide, a TP48 polypeptide, a TP65 polypeptide, and a TP50C polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

In some cases, an immunogenic composition of the present disclosure comprises: a) four TPs, where the four TPs are a TP156 polypeptide, a TP465 polypeptide, a TP48 polypeptide, and a TP65 polypeptide; and b) SLA formulated in: i) a stable emulsion; or ii) a liposomal composition comprising a saponin. In some cases, an immunogenic composition of the present disclosure comprises: a) four TPs, where the four TPs are a TP156 polypeptide, a TP465 polypeptide, a TP48 polypeptide, and a TP65 polypeptide; b) an HCV E1 polypeptide, an HCV E2 polypeptide, or an HCV E1/E2 heterodimer; and c) SLA formulated in: i) a stable emulsion; or ii) a liposomal composition comprising a saponin.

### Compositions Comprising T-Cell Epitope Fusion Polypeptides

As noted above, in some cases, an immunogenic composition of the present disclosure comprises a T-cell epitope fusion polypeptide, i.e., a fusion polypeptide comprising two or more of the T-cell epitope polypeptides described above. A T-cell epitope fusion polypeptide is also referred to herein as a “TP fusion polypeptide.”

In some cases, a TP fusion polypeptide does not include a linker peptide between any two TP polypeptides. In some cases, a TP fusion polypeptide comprises, between any two TP polypeptides, a linker peptide. Where a TP fusion polypeptide comprises one or more linker peptides, the one or more linker peptides are independently of a length of from 1 to 10 amino acids (e.g., 1, 2, 3, 4, 5, 6, 7, 8, 9, or 10 amino acids). Where a TP fusion polypeptide comprises one or more linker peptides, the one or more linker peptides are independently selected from, e.g., (GGGGS)n (SEQ ID NO:163), where n is an integer from 1 to 5 (e.g., n is 1, 2, 3, 4, or 5); AAAGG (SEQ ID NO:164); (EAAAK)n (SEQ ID NO:165); A(EAAAK)nA (SEQ ID NO:166); A(EAAAK)nALEA(EAAAK)nA (SEQ ID NO:167); (Lys-Pro)n (SEQ ID NO:168), (Glu-Pro)n (SEQ ID NO:169), (Thr-Pro-Arg)n (SEQ ID NO:170), or (Ala-Pro)n (SEQ ID NO:171), where n is an integer from 1 to 5 (e.g., n is 1, 2, 3, 4, or 5).

In some cases, a TP fusion polypeptide comprises only two TP polypeptides. For example, in some cases, a TP fusion polypeptide comprises a TP27 polypeptide and a TP35-NS3 polypeptide. As another example, in some cases, a TP fusion polypeptide comprises a TP27 polypeptide and a TP48 polypeptide. As another example, in some cases, a TP fusion polypeptide comprises a TP27 polypeptide and a TP50C polypeptide. As another example, in some cases, a TP fusion polypeptide comprises a TP35-NS3 polypeptide and a TP48 polypeptide. As another example, in some cases, a TP fusion polypeptide comprises a TP35-NS3 polypeptide and a TP50C polypeptide. As another example, in some cases, a TP fusion polypeptide comprises a TP50C polypeptide and a TP48 polypeptide. In some cases, a TP fusion polypeptide comprises only two TP polypeptides and does not include any other polypeptides.

In some cases, a TP fusion polypeptide comprises only three TP polypeptides. For example, in some cases, a TP fusion polypeptide comprises a TP27 polypeptide, a TP35-NS3 polypeptide, and a TP48 polypeptide. As another example, in some cases, a TP fusion polypeptide comprises a TP27 polypeptide, a TP48 polypeptide, and a TP50C polypeptide. As another example, in some cases, a TP fusion polypeptide comprises a TP35-NS3 polypeptide, a TP48 polypeptide, and a TP50C polypeptide. As another example, in some cases, a TP fusion polypeptide comprises a TP27 polypeptide, a TP35-NS3 polypeptide, and a TP50C polypeptide. In some cases, a TP fusion polypeptide comprises only three TP polypeptides and does not include any other polypeptides.

In some cases, a TP fusion polypeptide comprises four TP polypeptides (e.g., TP27, TP35-NS3, TP50C, and TP48). In some cases, a TP fusion polypeptide comprises all four of the above-described TP polypeptides and does not include any other polypeptides. In some cases, a TP fusion polypeptide has a total length of from 160 amino acids to 190 amino acids (e.g., from 160 amino acids to 165 amino acids, from 165 amino acids to 170 amino acids, from 170 amino acids to 175 amino acids, from 175 amino acids to 180 amino acids, from 180 amino acids to 185 amino acids, or from 185 amino acids to 190 amino acids). In some cases, a TP fusion polypeptide has a total length of 160 amino acids.

The four TP polypeptides (e.g., TP27, TP35-NS3, TP50C, and TP48) can be arranged in a TP fusion polypeptide in any order. 1) For example, in some cases, a TP fusion polypeptide comprises, in order from N-terminus to C-terminus: i) a TP35-NS3 polypeptide; ii) a TP27 polypeptide; iii) a TP48 polypeptide; and iv) a TP50C polypeptide. 2) As another example, in some cases, a TP fusion polypeptide comprises, in order from N-terminus to C-terminus: i) a TP35-NS3 polypeptide; ii) a TP27 polypeptide; iii) a TP50C polypeptide; and iv) a TP48 polypeptide. 3) As another example, in some cases, a TP fusion polypeptide comprises, in order from N-terminus to C-terminus: i) a TP35-NS3 polypeptide; ii) a TP48 polypeptide; iii) a TP27 polypeptide; and iv) a TP50C polypeptide. 4) As another example, in some cases, a TP fusion polypeptide comprises, in order from N-terminus to C-terminus: i) a TP35-NS3 polypeptide; ii) a TP48 polypeptide; iii) a TP50C polypeptide; and iv) a TP27 polypeptide. 5) As another example, in some cases, a TP fusion polypeptide comprises, in order from N-terminus to C-terminus: i) a TP35-NS3 polypeptide; ii) a TP50C polypeptide; iii) a TP27 polypeptide; and iv) a TP48 polypeptide. 6) As another example, in some cases, a TP fusion polypeptide comprises, in order from N-terminus to C-terminus: i) a TP35-NS3 polypeptide; ii) a TP50C polypeptide; iii) a TP48 polypeptide; and iv) a TP27 polypeptide. 7) As another example, in some cases, a TP fusion polypeptide comprises, in order from N-terminus to C-terminus: i) a TP27 polypeptide; ii) a TP35-NS3 polypeptide; iii) a TP50C polypeptide; and iv) a TP48 polypeptide. 8) As another example, in some cases, a TP fusion polypeptide comprises, in order from N-terminus to C-terminus: i) a TP27 polypeptide; ii) a TP35-NS3 polypeptide; iii) a TP48 polypeptide; and iv) a TP50C polypeptide. 9) As another example, in some cases, a TP fusion polypeptide comprises, in order from N-terminus to C-terminus: i) a TP27 polypeptide; ii) a TP48 polypeptide; iii) a TP35-NS3 polypeptide; and iv) a TP50C polypeptide. 10) As another example, in some cases, a TP fusion polypeptide comprises, in order from N-terminus to C-terminus: i) a TP27 polypeptide; ii) a TP48 polypeptide; iii) a TP50C polypeptide; and iv) a TP35-NS3 polypeptide. 11) As another example, in some cases, a TP fusion polypeptide comprises, in order from N-terminus to C-terminus: i) a TP27 polypeptide; ii) a TP50C polypeptide; iii) a TP48 polypeptide; and iv) a TP35-NS3 polypeptide. 12) As another example, in some cases, a TP fusion polypeptide comprises, in order from N-terminus to C-terminus: i) a TP27 polypeptide; ii) a TP50C polypeptide; iii) a TP35-NS3 polypeptide; and iv) a TP48 polypeptide. 13) As another example, in some cases, a TP fusion polypeptide comprises, in order from N-terminus to C-terminus: i) a TP48 polypeptide; ii) a TP27 polypeptide; iii) a TP35-NS3 polypeptide; and iv) a TP50C polypeptide. 14) As another example, in some cases, a TP fusion polypeptide comprises, in order from N-terminus to C-terminus: i) a TP48 polypeptide; ii) a TP27 polypeptide; iii) a TP50C polypeptide; and iv) a TP35-NS3 polypeptide. 15) As another example, in some cases, a TP fusion polypeptide comprises, in order from N-terminus to C-terminus: i) a TP48 polypeptide; ii) a TP35-NS3 polypeptide; iii) a TP27 polypeptide; and iv) a TP50C polypeptide. 16) As another example, in some cases, a TP fusion polypeptide comprises, in order from N-terminus to C-terminus: i) a TP48 polypeptide; ii) a TP35-NS3 polypeptide; iii) a TP50C polypeptide; and iv) a TP27 polypeptide. 17) As another example, in some cases, a TP fusion polypeptide comprises, in order from N-terminus to C-terminus: i) a TP48 polypeptide; ii) a TP50C polypeptide; iii) a TP27 polypeptide; and iv) a TP35-NS3 polypeptide. 18) As another example, in some cases, a TP fusion polypeptide comprises, in order from N-terminus to C-terminus: i) a TP48 polypeptide; ii) a TP50C polypeptide; iii) a TP35-NS3 polypeptide; and iv) a TP27 polypeptide. 19) As another example, in some cases, a TP fusion polypeptide comprises, in order from N-terminus to C-terminus: i) a TP50C polypeptide; ii) a TP27 polypeptide; iii) a TP35-NS3 polypeptide; and iv) a TP48 polypeptide. 20) As another example, in some cases, a TP fusion polypeptide comprises, in order from N-terminus to C-terminus: i) a TP50C polypeptide; ii) a TP27 polypeptide; iii) a TP48 polypeptide; and iv) a TP35-NS3 polypeptide. 21) As another example, in some cases, a TP fusion polypeptide comprises, in order from N-terminus to C-terminus: i) a TP50C polypeptide; ii) a TP35-NS3 polypeptide; iii) a TP27 polypeptide; and iv) a TP48 polypeptide. 22) As another example, in some cases, a TP fusion polypeptide comprises, in order from N-terminus to C-terminus: i) a TP50C polypeptide; ii) a TP35-NS3 polypeptide; iii) a TP48 polypeptide; and iv) a TP27 polypeptide. 23) As another example, in some cases, a TP fusion polypeptide comprises, in order from N-terminus to C-terminus: i) a TP50C polypeptide; ii) a TP48 polypeptide; iii) a TP27 polypeptide; and iv) a TP35-NS3 polypeptide. 24) As another example, in some cases, a TP fusion polypeptide comprises, in order from N-terminus to C-terminus: i) a TP50C polypeptide; ii) a TP48 polypeptide; iii) a TP35-NS3 polypeptide; and iv) a TP27 polypeptide.

As one non-limiting example, a TP fusion polypeptide comprises, in order from N-terminus to C-terminus: i) a TP35-NS3 polypeptide; ii) a TP27 polypeptide; iii) a TP48 polypeptide; and iv) a TP50C polypeptide. In some cases, a TP fusion polypeptide comprises an amino acid sequence having at least 80% (at least 80%, at least 85%, at least 90%, least 95%, least 98%, least 99%, or 100%) amino acid sequence identity to the amino acid sequence: KSTKVPAAYAAQGYKVLVLNPSVAATLGFGAYMSKLEQIKGGRHLIFCHSKKKCDELAAKLRI LRRHVGPGEGAVQWMNRLIAFASRGNHVSPTHYVPESDAAARVTQILGVYLLPRRGPRLGVRA TRKTSERSQPRGRRQPIPKARRSEGRSWAQPGYP (SEQ ID NO:172), where: i) KSTKVPAAYAAQGYKVLVLNPSVAATLGFGAYMSK (SEQ ID NO:79) is a TP35-NS3 polypeptide, ii) LEQIKGGRHLIFCHSKKKCDELAAKLR (SEQ ID NO:103) is a TP27 polypeptide; iii) ILRRHVGPGEGAVQWMNRLIAFASRGNHVSPTHYVPESDAAARVTQIL (SEQ ID NO:136) is a TP48 polypeptide, and iv) GVYLLPRRGPRLGVRATRKTSERSQPRGRRQPIPKARRSEGRSWAQPGYP (SEQ ID NO:87) is a TP50C polypeptide. In some cases, a TP fusion polypeptide has a total length of from 160 amino acids to 190 amino acids (e.g., from 160 amino acids to 165 amino acids, from 165 amino acids to 170 amino acids, from 170 amino acids to 175 amino acids, from 175 amino acids to 180 amino acids, from 180 amino acids to 185 amino acids, or from 185 amino acids to 190 amino acids). In some cases, a TP fusion polypeptide has a total length of 160 amino acids.

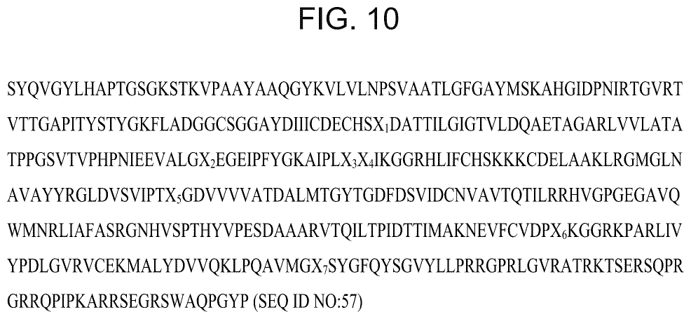

In some cases, a TP fusion polypeptide comprises a TP240 polypeptide, a TP48 polypeptide, a TP65 polypeptide, and a TP50C polypeptide, and does not include any other polypeptides. The total length of the TP fusion polypeptide can be from 410 amino acids to 415 amino acids (e.g., 410, 411, 412, 413, 414, or 415 amino acids). In some cases, the TP fusion polypeptide has a length of 410 amino acids. The four TP polypeptides can be arranged in a TP fusion polypeptide in any order. For example, in some cases, a TP fusion polypeptide comprises, in order from N-terminus to C-terminus: i) a TP240 polypeptide; ii) a TP48 polypeptide; and iii) a TP65 polypeptide; iv) a TP50C polypeptide. As an example, the TP fusion polypeptide can comprise an amino acid sequence having at least about 90%, at least about 95%, at least about 98%, at least about 99%, or 100%, amino acid sequence identity, to the amino acid sequence depicted in FIG. 10, where X₁ is T, V, or Q, X₂ is T, N, Q, H, or S, X₃ is E, S, or A, X₄ is V, T, Y, F, or L, X₅ is S, Q, or T, X₆ is E, T, or V, and X₇ is S, A, or P; where the TP fusion polypeptide has a length of from 410 amino acids to 415 amino acids (e.g., 410, 411, 412, 413, 414, or 415 amino acids).

### HCV E1 Polypeptides, HCV E2 Polypeptides, and HCV E1/E2 Heterodimers

As noted above, in some cases, an immunogenic composition of the present disclosure comprises an HCV E1 polypeptide, an HCV E2 polypeptide, or an HCV E1/E2 heterodimer. E1 polypeptides

An HCV E1 polypeptide suitable for inclusion in an immunogenic composition of the present disclosure, or for inclusion in an HCV E1/E2 heterodimer present in an immunogenic composition of the present disclosure, can have a length of from about 100 amino acids (aa) to about 150 aa, from about 150 aa to about 175 aa, from about 175 aa to about 195 aa, from about 131 aa to about 175 aa, or from about 175 aa to about 193 aa. In some cases, an HCV E1 polypeptide is an HCV E1 ectodomain polypeptide. In some cases, an HCV E1 polypeptide is a full-length HCV E1 polypeptide.

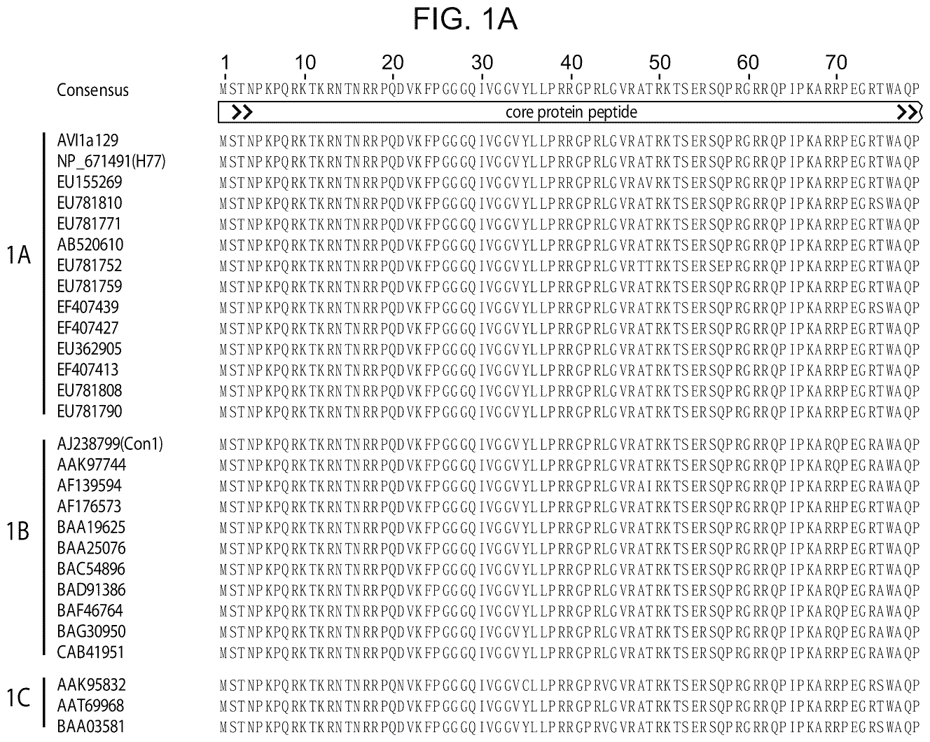

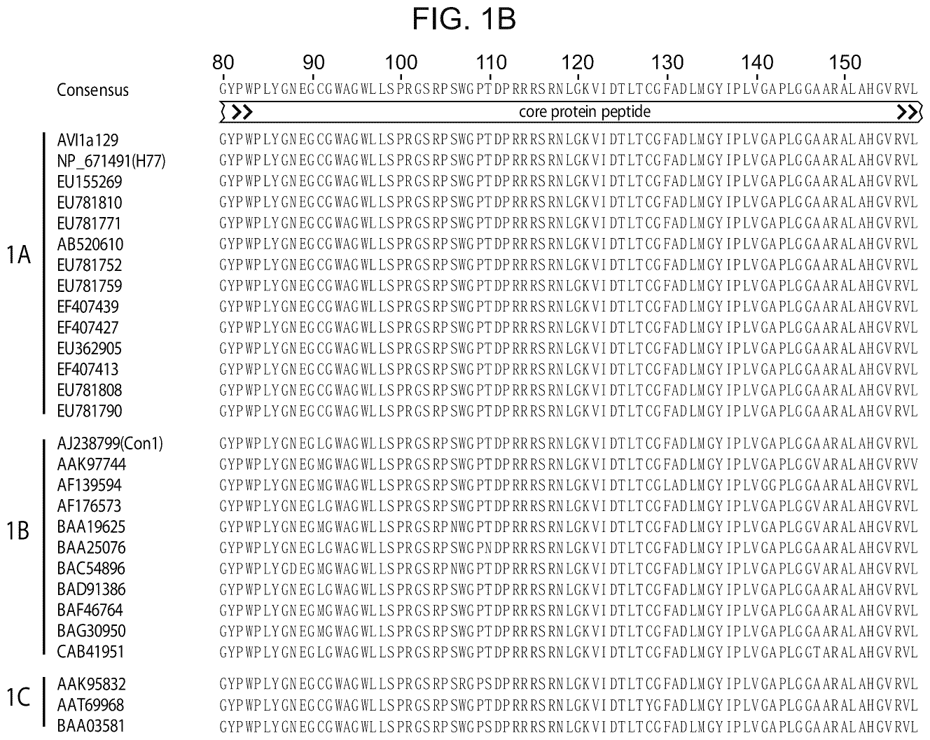

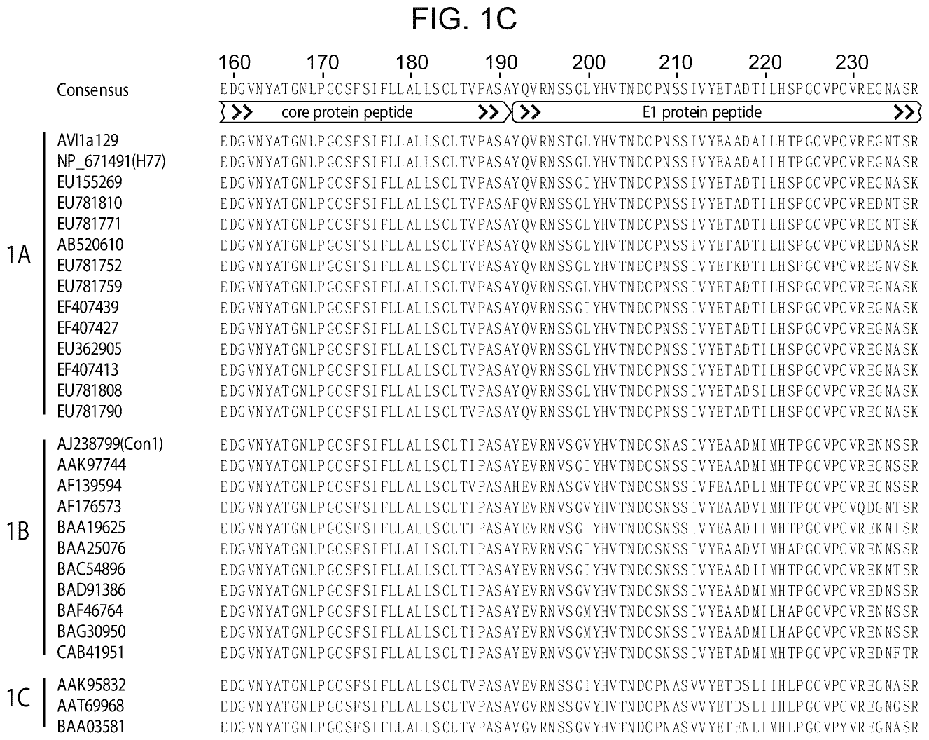

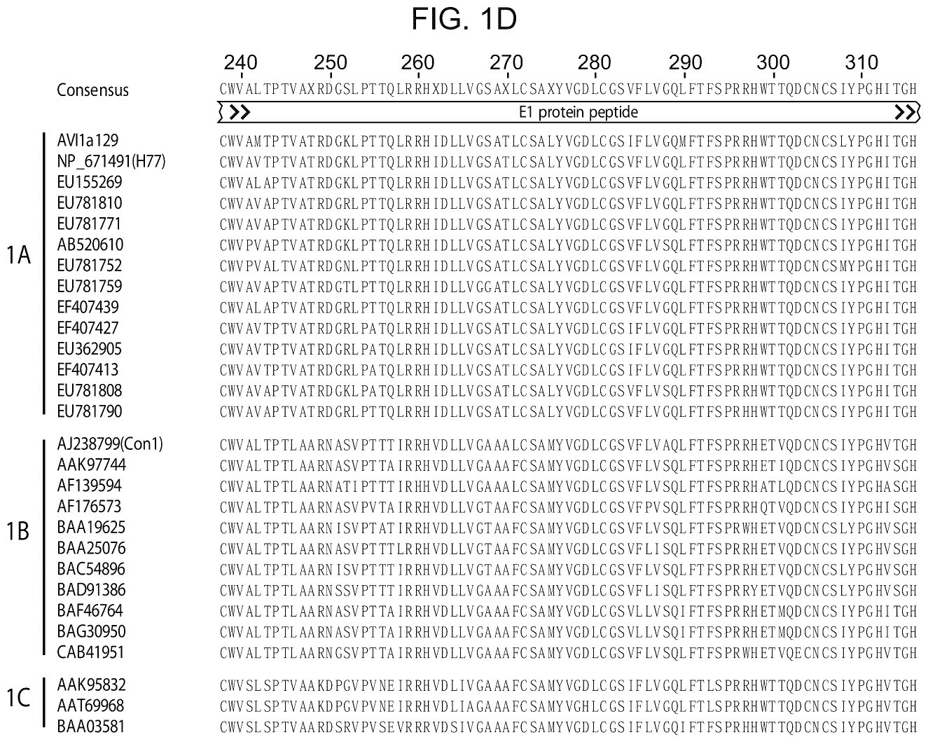

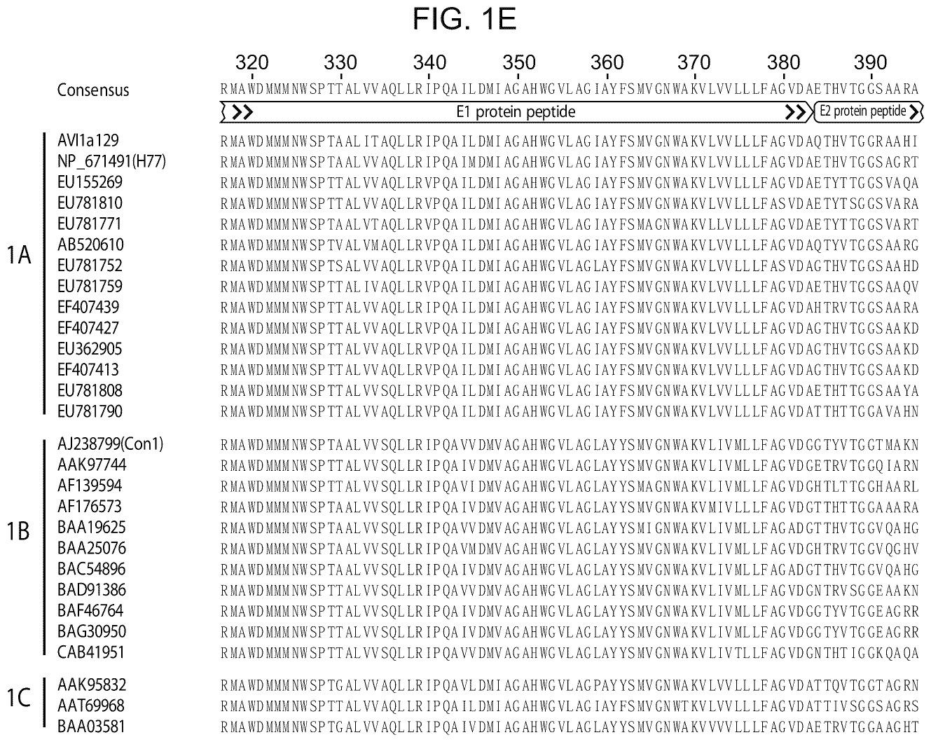

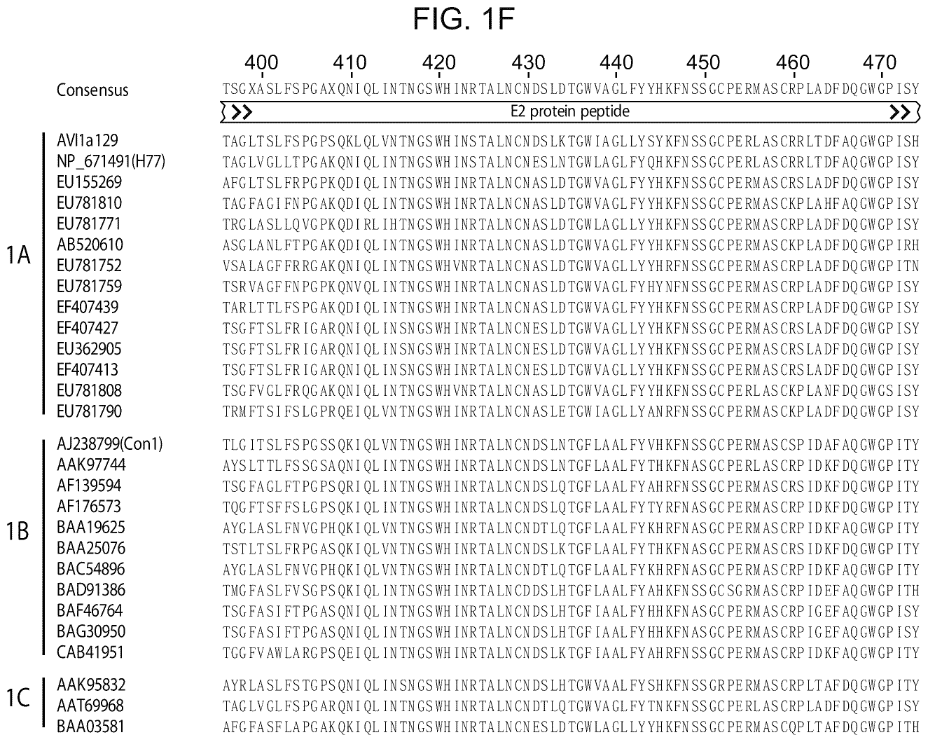

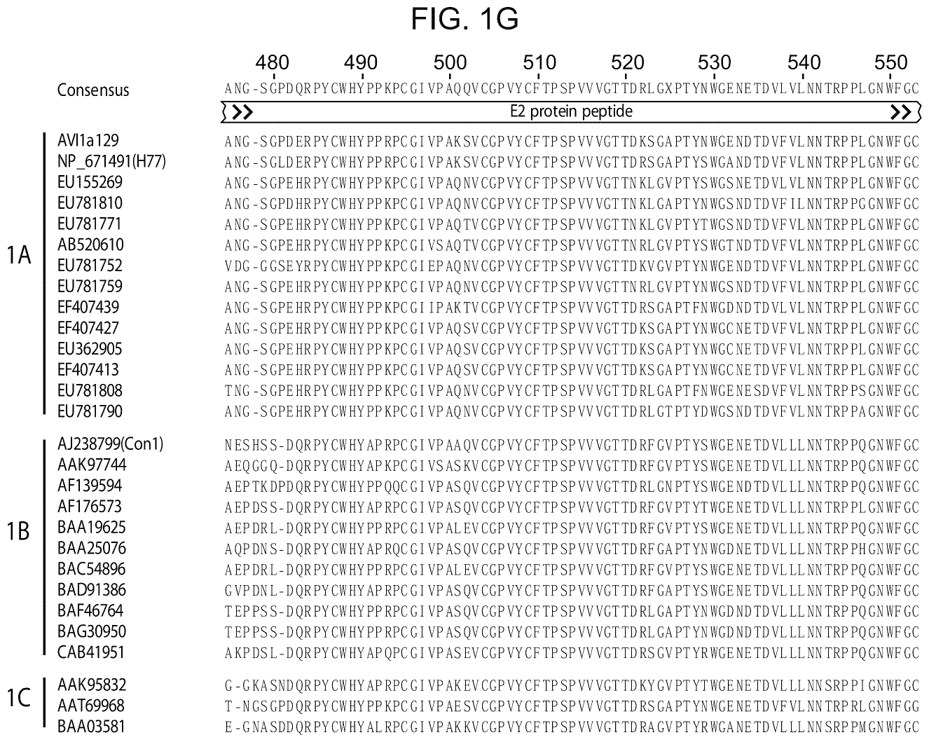

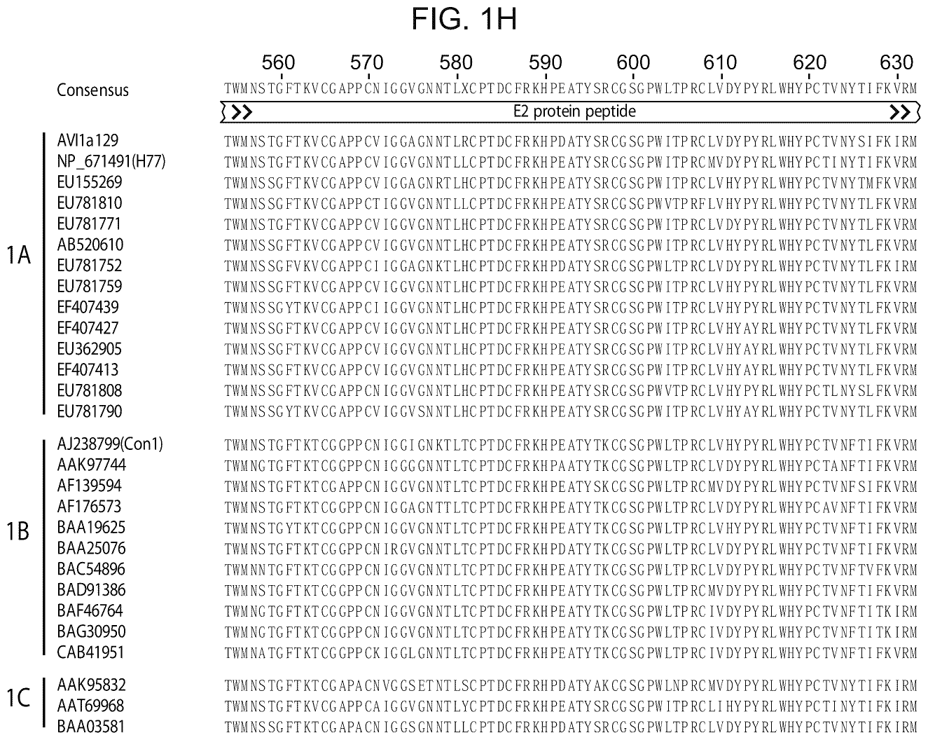

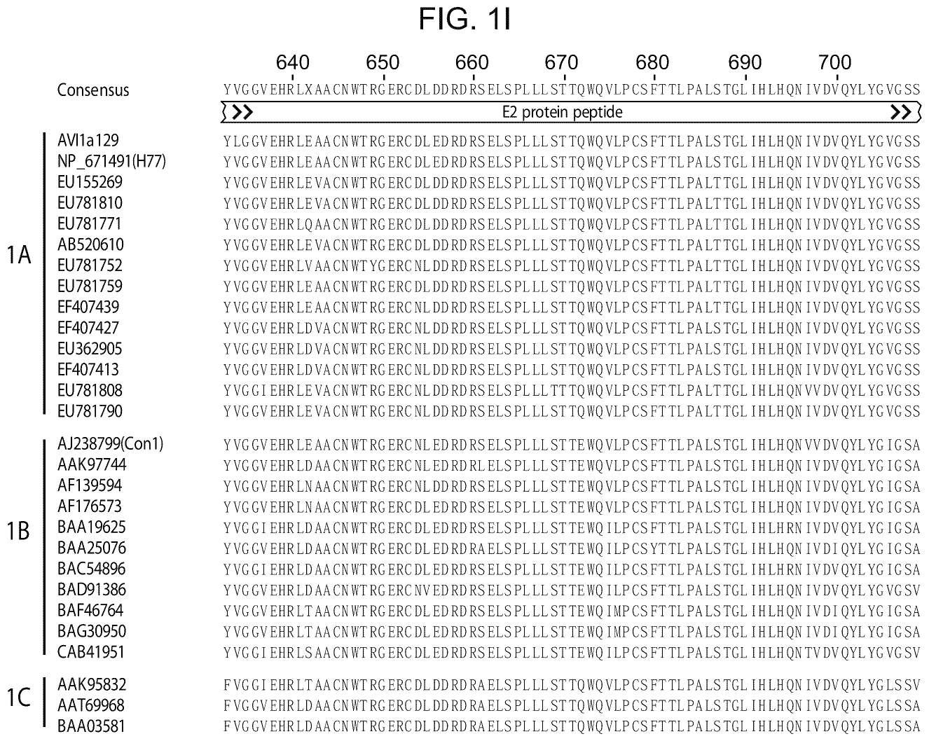

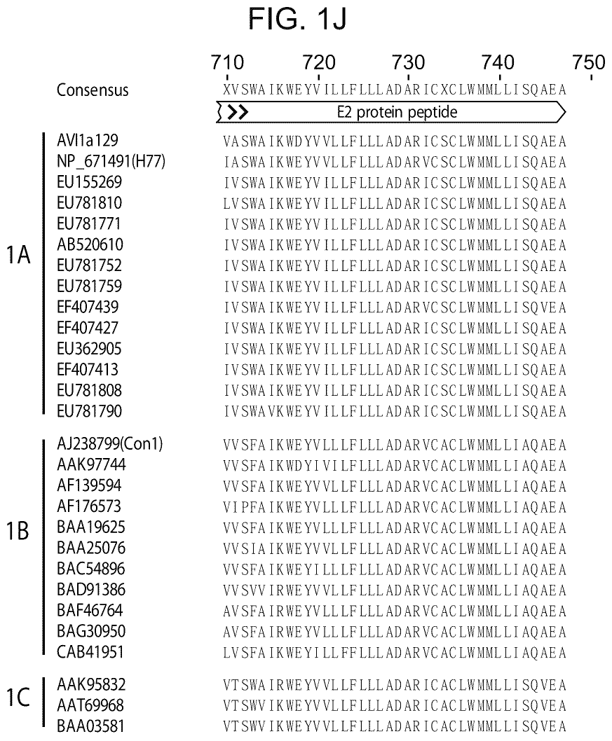

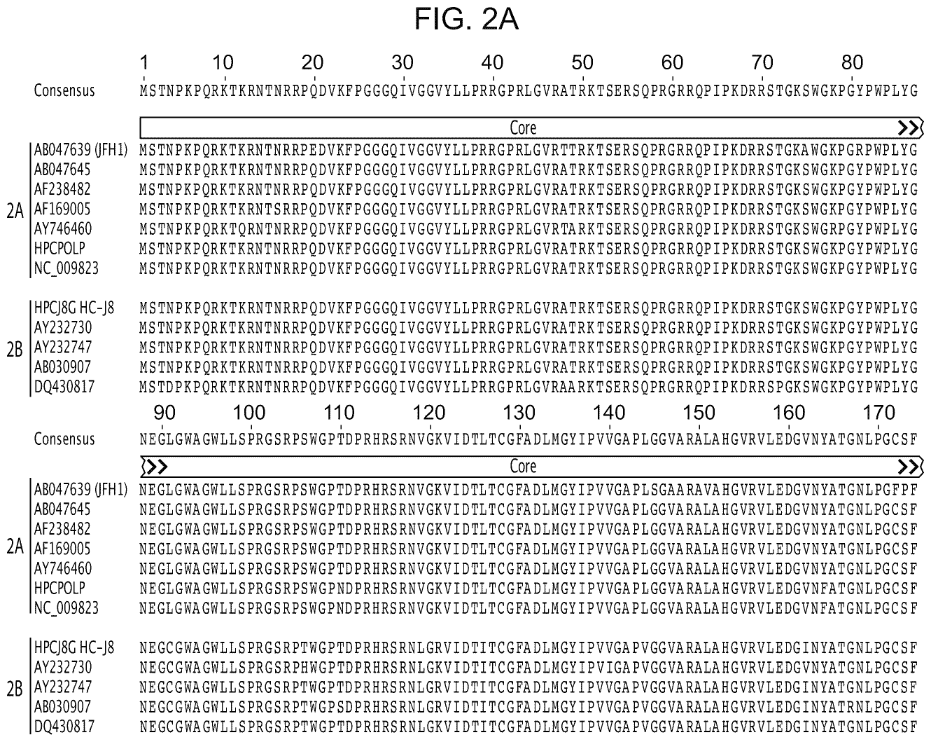

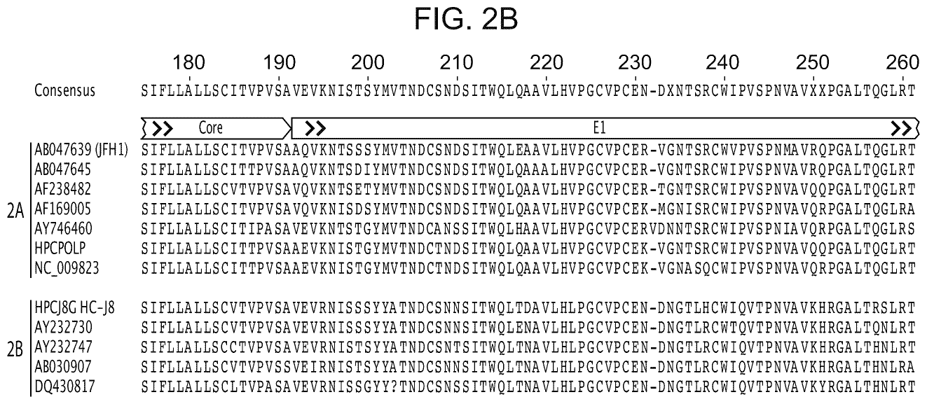

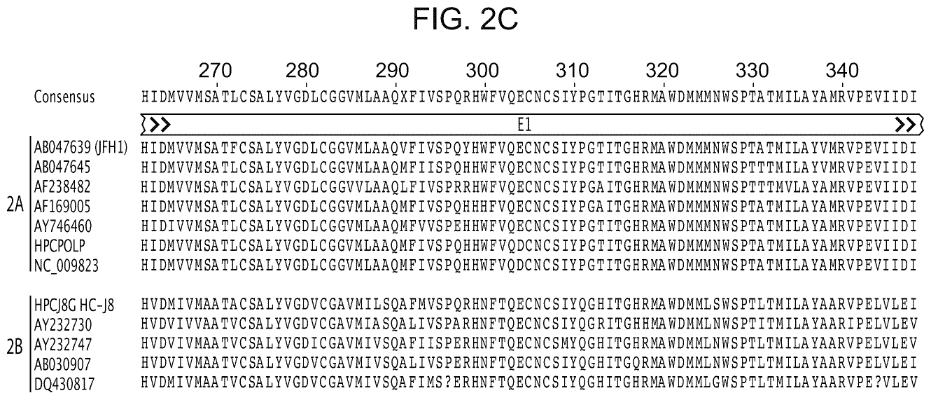

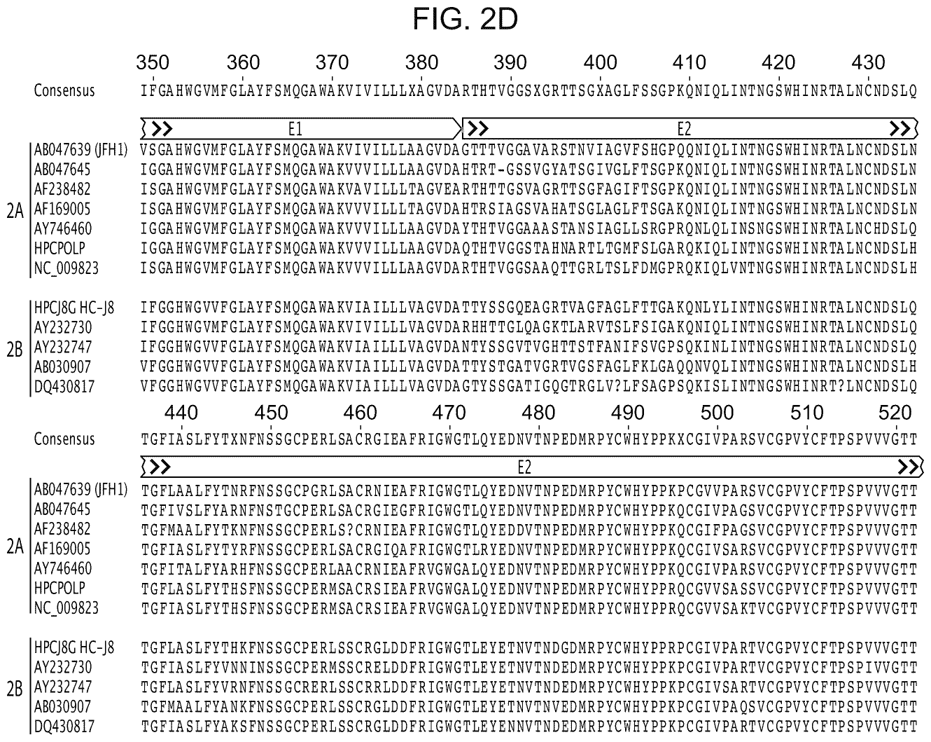

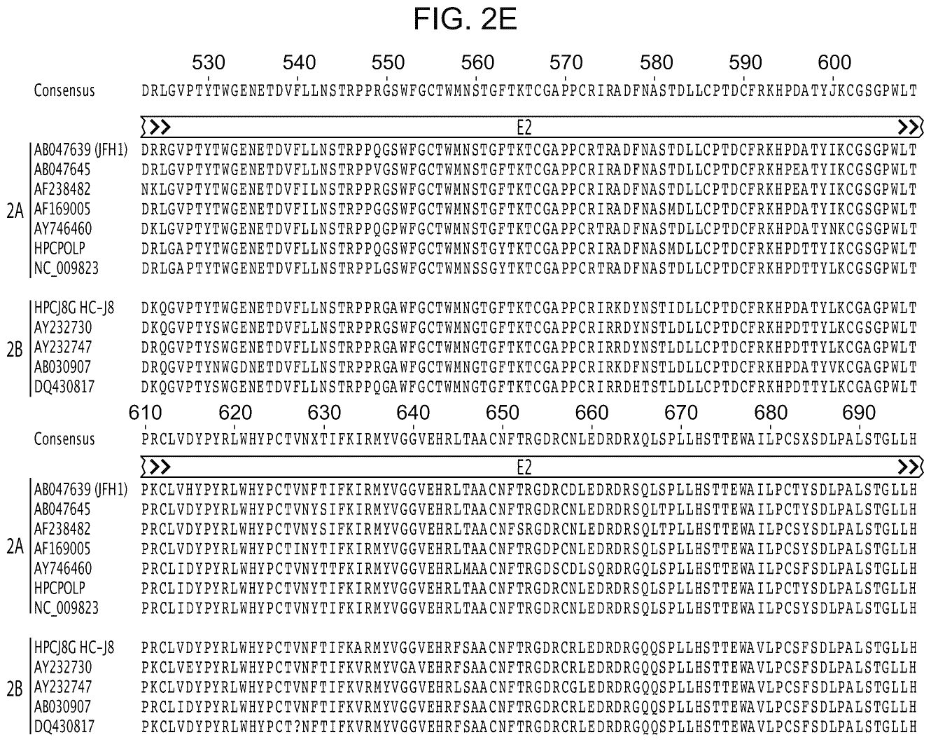

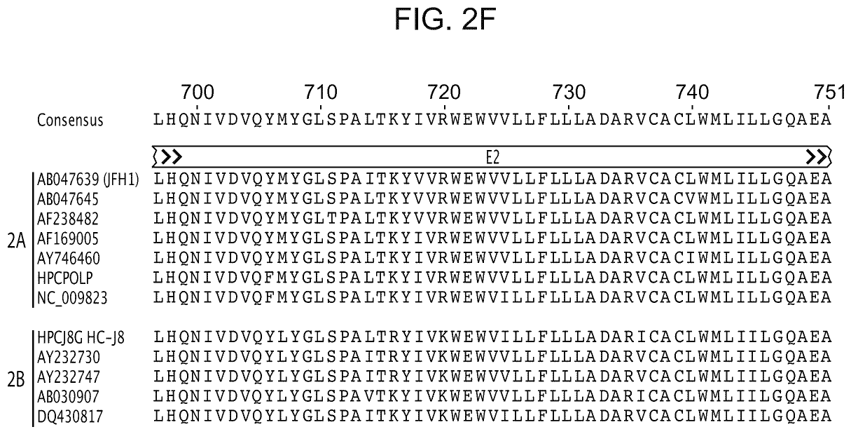

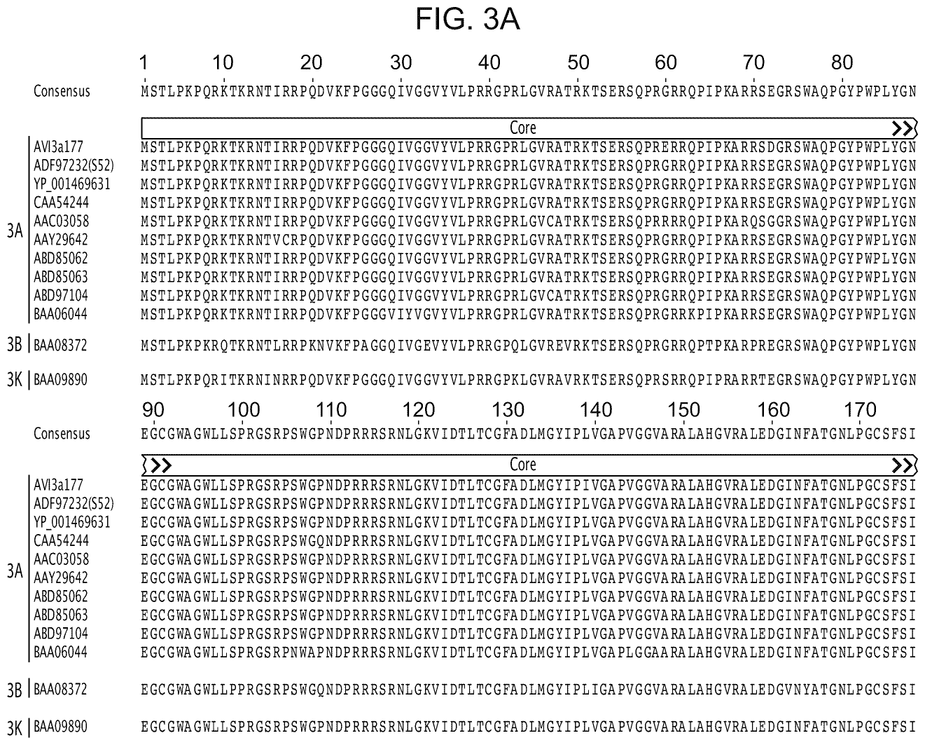

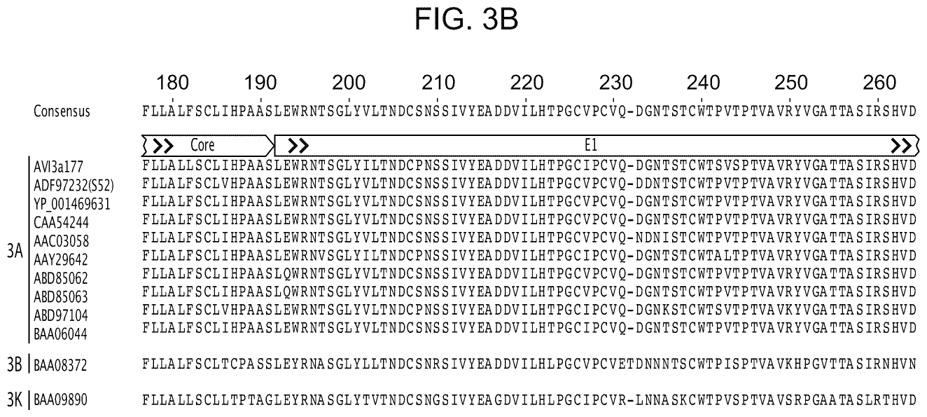

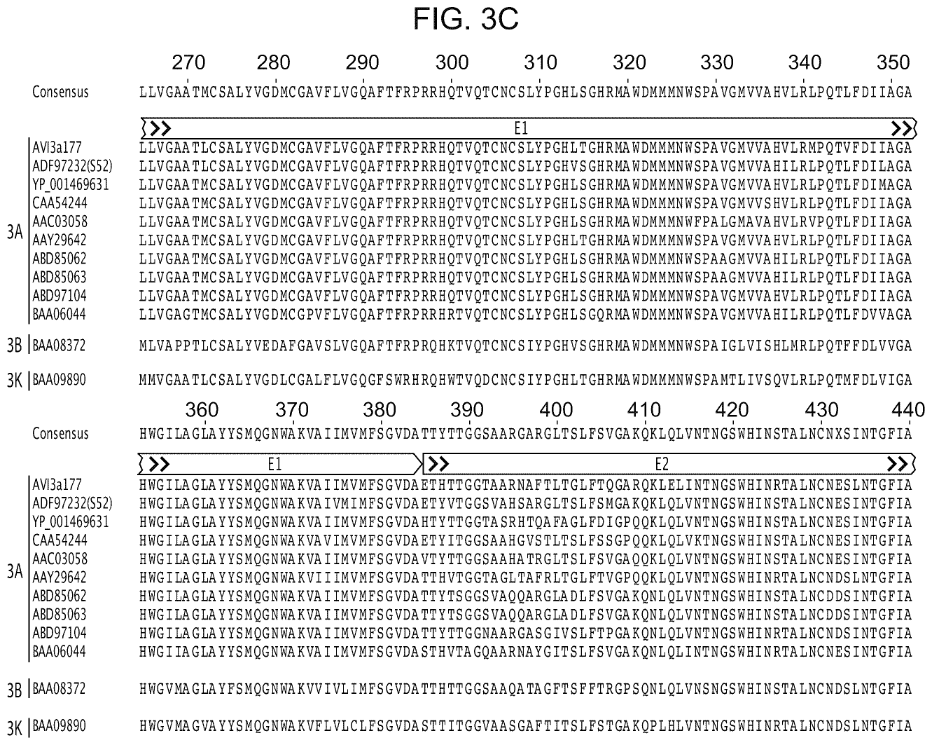

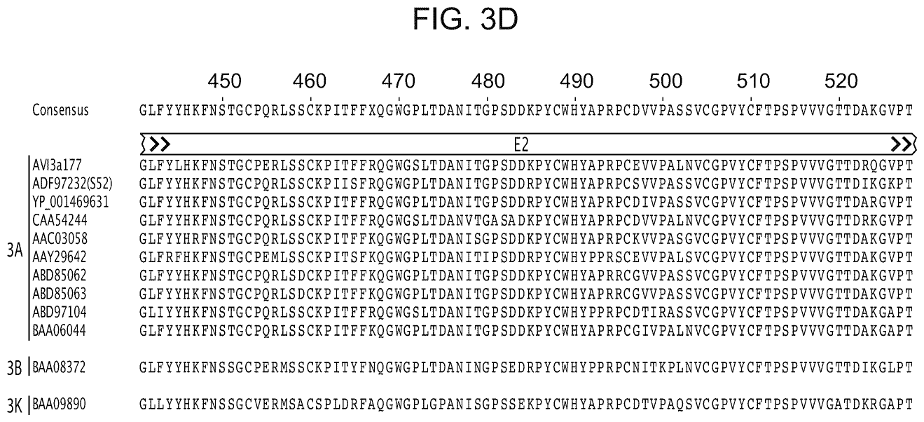

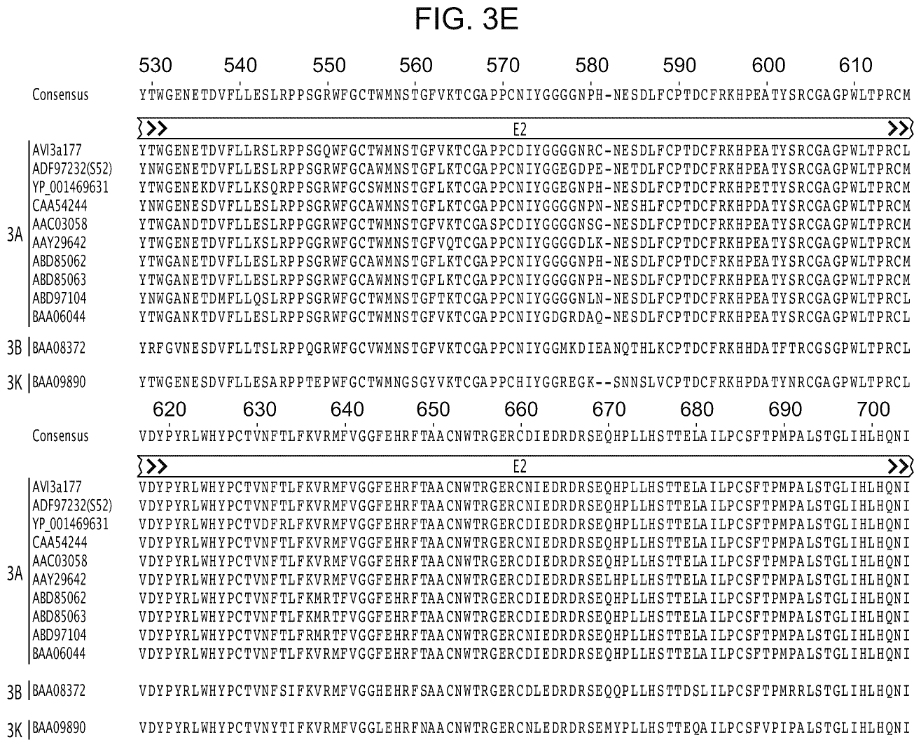

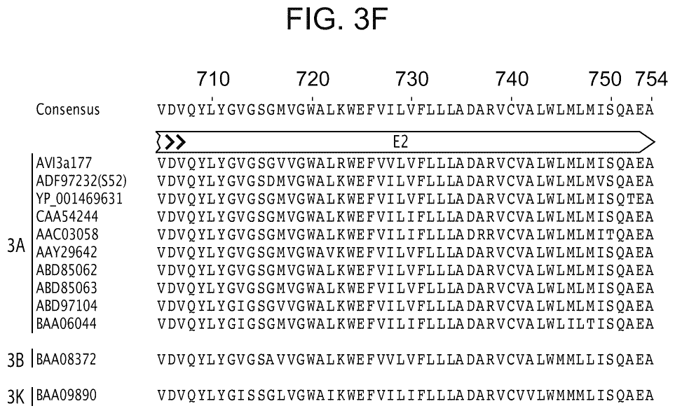

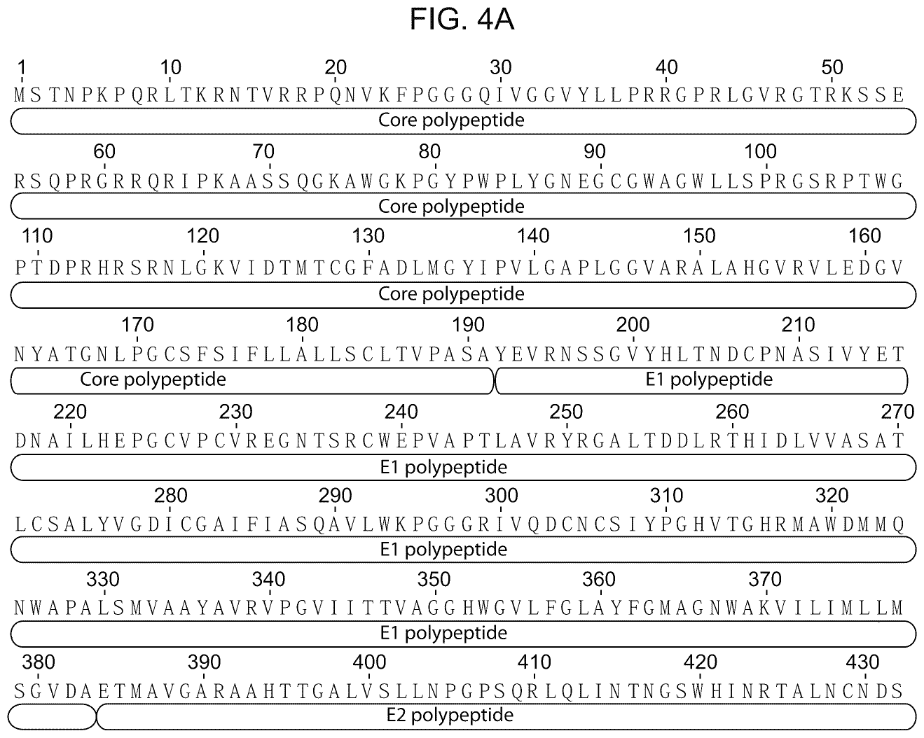

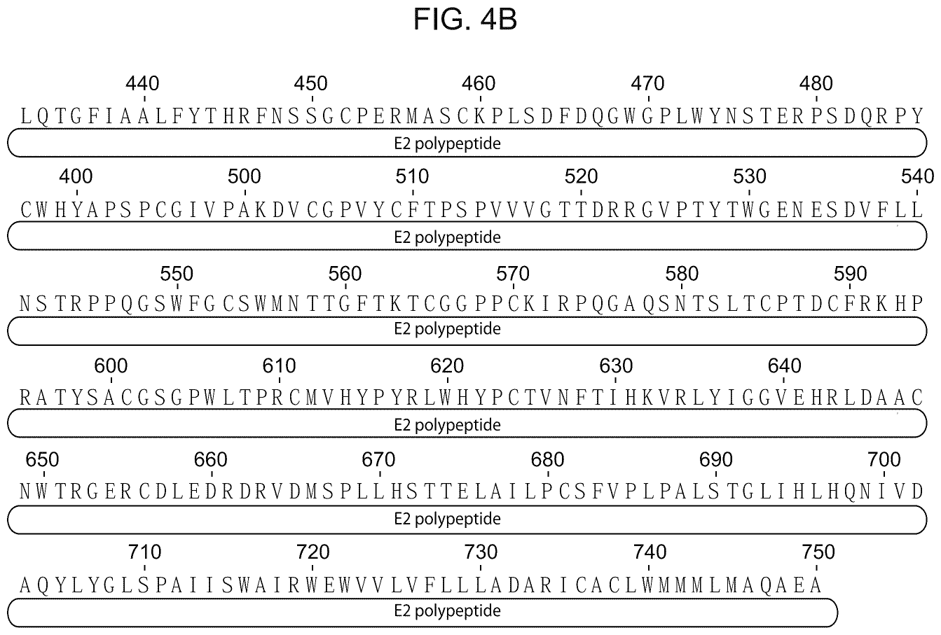

In FIG. 1A-1J, the amino acid sequence of E1 is amino acid 192 to amino acid 383. In FIG. 2A-2F, the amino acid sequence of E1 is amino acid 192 to amino acid 383. In FIG. 3A-3F, the amino acid sequence of E1 is amino acid 192 to amino acid 384. In FIG. 4A-4B, the amino acid sequence of E1 is amino acid 192 to amino acid 383. Amino acids at around 170 through approximately 191 serve as a signal sequence for E1. As used herein, “E1 polypeptide” includes a precursor E1 protein, including the signal sequence; includes a mature E1 polypeptide which lacks this sequence; and includes an E1 polypeptide with a heterologous signal sequence. An E1 polypeptide can include a C-terminal membrane anchor sequence which occurs at approximately amino acid positions 360-383 (see, e.g., WO 96/04301). In some cases, a suitable E1 polypeptide lacks a C-terminal portion that includes a transmembrane region. For example, in some cases, a suitable E1 polypeptide lacks the C-terminal portion from amino acid 330 to amino acid 384, or from amino acid 360 to amino acid 384. E1 polypeptides can be an E1 polypeptide of any genotype, subtype or isolate of HCV. E1 polypeptides of genotype 1 and E1 polypeptides of genotype 3 are included in an E1/E2 heterodimer of the present disclosure.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 1A-1J, FIG. 2A-2F, FIG. 3A-3F, or FIG. 4A-4B.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 1A-1J. For example, an E1 polypeptide of genotype 1A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 1A and depicted in FIG. 1A-1J. For example, an E1 polypeptide of genotype 1B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 1B and depicted in FIG. 1A-1J. For example, an E1 polypeptide of genotype 1C can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 1C and depicted in FIG. 1A-1J.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 2A-2F. For example, an E1 polypeptide of genotype 2A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 2A and depicted in FIG. 2A-2F. For example, an E1 polypeptide of genotype 2B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 2B and depicted in FIG. 2A-2F.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the consensus E1 polypeptide amino acid sequence depicted in FIG. 3A-3F.

An E1 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 4A-4B. For example, an E1 polypeptide of genotype 7A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of the amino acid sequence depicted in FIG. 4A-4B. E2 polypeptides

An E2 polypeptide suitable for inclusion in an immunogenic composition of the present disclosure, or for inclusion in an HCV E1/E2 heterodimer present in an immunogenic composition of the present disclosure, can have a length of from about 200 amino acids (aa) to about 250 aa, from about 250 aa to about 275 aa, from about 275 aa to about 300 aa, from about 300 aa to about 325 aa, from about 325 aa to about 350 aa, or from about 350 aa to about 365 aa. In some cases, an HCV E2 polypeptide is an HCV E2 ectodomain polypeptide. In some cases, an HCV E2 polypeptide is a full-length HCV E2 polypeptide.

In FIG. 1A-1J, the amino acid sequence of E2 is amino acid 384 to amino acid 746. In FIG. 2A-2B, the amino acid sequence of E2 is amino acid 384 to amino acid 751. In FIG. 3A-3F, the amino acid sequence of E2 is amino acid 385 to amino acid 754. In FIG. 4A-4B, the amino acid sequence of E2 is amino acid 384 to amino acid 750. As used herein, an “E2 polypeptide” includes a precursor E2 protein, including the signal sequence; includes a mature E2 polypeptide which lacks this sequence; and includes an E2 polypeptide with a heterologous signal sequence. An E2 polypeptide can include a C-terminal membrane anchor sequence which occurs at approximately amino acid positions 715-730 and may extend as far as approximately amino acid residue 746 (see, Lin et al., J. Virol. (1994) 68:5063-5073).

In some cases, a E2 polypeptide lacks a portion of its C-terminal region, e.g., from about amino acid 715 to the C-terminus; from about amino acid 625 to the C-terminus; from about amino acid 661 to the C-terminus; from about amino acid 655 to the C-terminus; from about amino acid 500 to the C-terminus, where the amino acid numbering is with reference to the numbering in FIG. 1A-1J. See, e.g., U.S. Pat. No. 6,521,423.

An E2 polypeptide suitable for inclusion in an immunogenic composition of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 1A-1J, FIG. 2A-2F, FIG. 3A-3F, or FIG. 4A-4B. An E2 polypeptide suitable for inclusion in an immunogenic composition of the present disclosure can comprise an amino acid sequence having at least about 20%, at least about 25%, at least about 30%, at least about 35%, at least about 40%, at least about 45%, at least about 50%, at least about 60%, at least about 70%, or at least about 75%, amino acid sequence identity to an amino acid sequence of an E2 polypeptide depicted in FIG. 1A-1J, FIG. 2A-2F, FIG. 3A-3F, or FIG. 4A-4B.

An E2 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 1A-1J. For example, an E2 polypeptide of genotype 1 can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence depicted in FIG. 1A-1J. For example, an E2 polypeptide of genotype 1A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence identified as 1A and depicted in FIG. 1A-1J. For example, an E2 polypeptide of genotype 1B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence identified as 1B and depicted in FIG. 1A-1J. For example, an E2 polypeptide of genotype 1C can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence identified as 1C and depicted in FIG. 1A-1J.

An E2 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 2A-2F. For example, an E2 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-751 of an amino acid sequence depicted in FIG. 2A-2F. For example, an E2 polypeptide of genotype 2A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-751 of the “consensus” amino acid sequence depicted in FIG. 2A-2F. For example, an E2 polypeptide of genotype 2B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-751 of the “consensus” amino acid sequence depicted in FIG. 2A-2F.

An E2 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 3A-3F. For example, an E2 polypeptide of genotype 3 can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of an amino acid sequence depicted in FIG. 3A-3F. For example, an E2 polypeptide of genotype 3A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of an amino acid sequence identified as 3A and depicted in FIG. 3A-3F. For example, an E2 polypeptide of genotype 3B can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of the amino acid sequence identified as 3B and depicted in FIG. 3A-3F. For example, an E2 polypeptide of genotype 3K can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of the amino acid sequence identified as 3K and depicted in FIG. 3A-3F.

An E2 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the amino acid sequence of the E2 polypeptide depicted in FIG. 4A-4B. For example, an E2 polypeptide of genotype 7A can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-750 of the amino acid sequence depicted in FIG. 4A-4B.

### HCV E1E2 Heterodimers

HCV E1/E2 heterodimers suitable for use in an immunogenic composition of the present disclosure include HCV E1/E2 heterodimers comprising wild-type HCV E1 polypeptides; HCV E1/E2 heterodimers comprising wild-type HCV E2 polypeptides; HCV E1/E2 heterodimers comprising variant HCV E1 polypeptides; and HCV E1/E2 heterodimers comprising variant HCV E2 polypeptides. HCV E2 polypeptides suitable for use in an immunogenic composition of the present disclosure include wild-type E2 polypeptides and variant E2 polypeptides. HCV E1 polypeptides suitable for use in an immunogenic composition of the present disclosure include wild-type E1 polypeptides and variant E1 polypeptides. An HCV E1/E2 heterodimer can include any of the HCV E1 polypeptides discussed above and any of the HCV E2 polypeptides discussed above.

### Containers

The present disclosure provides a container comprising an immunogenic composition of the present disclosure. The container can be, e.g., a syringe, an ampoule, and the like. In some cases, the container is sterile. In some cases, both the container and the immunogenic composition are sterile.

### Methods of Inducing an Immune Response

The present disclosure provides a method of inducing an immune response (e.g., a protective immune response) to at least one HCV genotype in a mammalian subject, where the method comprises administering an effective among of an immunogenic composition of the present disclosure to the individual.

In some cases, a prime-boost vaccine protocol is used. In some cases, a first (priming) immunogenic composition is administered, and, after a time, a second (booster) immunogenic composition is administered. In some cases, the first immunogenic composition and the second immunogenic composition are the same. In some cases, the first immunogenic composition and the second immunogenic composition are different. For example, in some cases, the first immunogenic composition comprises a first TP fusion polypeptide; and the second immunogenic composition comprises a second TP fusion polypeptide that has a different amino acid sequence from the first TP fusion polypeptide. A second immunogenic composition can be administered at a time period of from 1 day to 1 year following administration of the first immunogenic composition. For example, a second immunogenic composition can be administered at a time period of from 1 day to 1 week, from 1 week to 2 weeks, from 2 weeks to 1 month, from 1 month to 2 months, from 2 months to 6 months, or from 6 months to 1 year following administration of the first immunogenic composition.

### General Considerations

An immunogenic composition of the present disclosure is generally administered to a human subject who: i) has an HCV infection; or ii) is at risk of acquiring an HCV infection (e.g., is at greater risk than the general population of acquiring an HCV infection); or iii) is naïve with respect to HCV infection, so as to prevent or at least partially arrest the development of disease and its complications. An amount adequate to accomplish this is defined as a “therapeutically effective dose” or a “therapeutically effective amount.” “Prophylactic” use of a subject immunogenic composition generally refers to administration to an individual who has not been infected with HCV (e.g., a “naïve” individual). “Therapeutic” use of a subject immunogenic composition can refer to “prophylactic” use (administration to an individual who has not been infected with HCV) and/or to administration to an individual who has an HCV infection. A “therapeutically effective amount” of an immunogenic composition of the present disclosure, can be an amount that, when administered in one or more doses to an individual who is not infected with HCV, is effective to induce an immune response in the individual to HCV. A “therapeutically effective amount” of an immunogenic composition of the present disclosure, can be an amount that, when administered in one or more doses to an individual who is infected with HCV, is effective to enhance an immune response in the individual to HCV.

Amounts effective for therapeutic use will depend on, e.g., the manner of administration, the weight and general state of health of the patient, and the judgment of the prescribing physician. Single or multiple doses of a subject immunogenic composition can be administered depending on the dosage and frequency required and tolerated by the patient, and route of administration.

In some cases, an effective amount of an immunogenic composition of the present disclosure is an amount that, when administered to an individual in one or more doses, is effective to induce an antibody response (e.g., a neutralizing antibody response) to HCV in the individual. For example, antibody to HCV (e.g., extracellular HCV), and/or to an HCV-infected cell, can be induced.

An effective amount of an immunogenic composition of the present disclosure can be an amount that, when administered to an individual in one or more doses, is effective to induce a neutralizing antibody response to HCV of a variety of genotypes (e.g., genotype 1; genotype 3; etc.). A neutralizing antibody response reduces binding of HCV to one or more host receptors for HCV and inhibits entry of HCV into a cell.

In some cases, an effective amount (e.g., a therapeutically effective amount) of an immunogenic composition of the present disclosure is an amount that, when administered to an individual in one or more doses, is effective to induce a cytotoxic T lymphocyte (CTL) response to HCV (e.g., a CD8⁺ T cell response). For example, a CTL response to an HCV-infected cell can be induced.

In some cases, an effective amount (e.g., a therapeutically effective amount) of an immunogenic composition of the present disclosure is an amount that, when administered to an individual in one or more doses, is effective to induce a helper T lymphocyte (e.g., CD4⁺ T cell) to HCV in an individual.

In some cases, an effective amount (e.g., a therapeutically effective amount) of an immunogenic composition of the present disclosure is an amount that, when administered to an individual in one or more doses, is effective to induce an antibody response (e.g., a neutralizing antibody response) and/or a CTL response and/or a helper T cell response to HCV genotype 1. In some cases, an effective amount (e.g., a therapeutically effective amount) of an immunogenic composition of the present disclosure is an amount that, when administered to an individual in one or more doses, is effective to induce an antibody response (e.g., a neutralizing antibody response) and/or a CTL response and/or a helper T cell response to HCV genotype 3. In some cases, an effective amount (e.g., a therapeutically effective amount) of an immunogenic composition of the present disclosure is an amount that, when administered to an individual in one or more doses, is effective to induce an antibody response (e.g., a neutralizing antibody response) and/or a CTL response and/or a helper T cell response to HCV genotype 1 and HCV genotype 3. In some cases, an effective amount (e.g., a therapeutically effective amount) of an immunogenic composition of the present disclosure is an amount that, when administered to an individual in one or more doses, is effective to induce an antibody response (e.g., a neutralizing antibody response) and/or a CTL response and/or a helper T cell response to HCV of any genotype. In some cases, an effective amount (e.g., a therapeutically effective amount) of an immunogenic composition of the present disclosure is an amount that, when administered to an individual in one or more doses, is effective to induce a CD8⁺ T cell response to HCV genotypes 1-6. In some cases, an effective amount (e.g., a therapeutically effective amount) of an immunogenic composition of the present disclosure is an amount that, when administered to an individual in one or more doses, is effective to induce a CD4⁺ T cell response to HCV genotypes 1-6.

An immunogenic composition of the present disclosure is generally administered in an amount effective to elicit an immune response, e.g., a humoral immune response (e.g., an antibody response) and/or a CTL response, in the mammalian subject. Effective amounts of E1/E2, E1, or E2 polypeptides for immunization will vary, and can generally range from about 1 pg to 100 μg per 70 kg patient, e.g., from about 5 μg/70 kg to about 50 pg/70 kg. Substantially higher dosages (e.g. 10 mg to 100 mg or more) of an HCV E1/E2, E1, or E2 polypeptide may be suitable in oral, nasal, or topical administration routes. In some cases, a dose of an immunogenic composition of the present disclosure comprises an HCV E1/E2 heterodimer in an amount of from 4 μg to 100 μg. For example, in some cases, a dose of an immunogenic composition of the present disclosure comprises an HCV E1/E2 heterodimer in an amount of from 4 μg to 5 μg, from 5 μg to 10 μg, from 10 μg to 15 μg, from 15 μg to 20 μg, from g to 25 μg, from 25 μg to 30 μg, from 30 μg to 40 μg, from 40 μg to 50 μg, from 50 μg to 60 μg, from 60 μg to 70 μg, from 70 μg to 80 μg, from 80 μg to 90 μg, or from 90 μg to 100 μg. In some cases, a dose of an immunogenic composition of the present disclosure comprises an HCV E1/E2 heterodimer in an amount of from about 100 μg to about 200 μg.

In some cases, the amount of T-cell epitope polypeptide in a given dose ranges from 0.1 g to 125 μg; e.g., a suitable dose of T-cell epitope polypeptide ranges from 0.1 μg to 0.5 μg, from 0.5 g to 1 μg, from 1 μg to 5 μg, from 5 μg to 10 μg, from 10 μg to 15 μg, from 15 μg to 20 μg, from 20 g to 25 μg, from 25 μg to 30 μg, from 30 μg to 40 μg, from 40 μg to 50 μg, from 50 μg to 60 μg, from 60 g to 75 μg, from 75 μg to 100 μg, or from 100 μg to 125 μg. In some cases, where two or more different T-cell epitope polypeptides are included in an immunogenic composition, a dose will include from 0.5 g to 125 μg of each T-cell epitope polypeptide. In some cases, where two (or more) different T-cell epitope polypeptides are included in an immunogenic composition, a dose can include from 1 μg to 10 g (e.g., 1 μg, 2 μg, 3 μg, 4 μg, 5 μg, 6 μg, 7 μg, 8 μg, 9 μg, or 10 μg) of each T-cell epitope polypeptide. As one non-limiting example, where an immunogenic composition of the present disclosure includes 2 or more different T-cell epitope polypeptides, the composition can include 1 μg of each different T-cell epitope polypeptide. As another non-limiting example, where an immunogenic composition of the present disclosure includes 2 or more different T-cell epitope polypeptides, the composition can include 3 μg of each different T-cell epitope polypeptide. As another non-limiting example, where an immunogenic composition of the present disclosure includes 2 or more different T-cell epitope polypeptides, the composition can include 6 μg of each different T-cell epitope polypeptide.

In some cases, a single dose of an immunogenic composition of the present disclosure comprises: a) an HCV E1/E2 heterodimer in an amount of from about 10 μg to about 15 μg (e.g., 10 μg, 11 μg, 12 μg, 13 μg, 14 μg, or 15 μg E1/E2 heterodimer); and b) two or more different T-cell epitope polypeptides in an amount of from about 5 μg each different T-cell epitope polypeptide to about 15 g each different T-cell epitope polypeptide (e.g., 5 μg, 6 μg, 7 μg, 8 μg, 9 μg, 10 μg, 11 μg, 12 μg, 13 μg, 14 μg, or 15 μg each different T-cell epitope polypeptide. As one non-limiting example, in some cases, a single dose of an immunogenic composition of the present disclosure comprises: a) 15 μg E1/E2 heterodimer; and b) 40 μg T-cell epitope polypeptides (10 μg each of TP35-NS3, TP50C, TP27, and TP48).

In some cases, a dose of an immunogenic composition of the present disclosure comprises an E1/E2 heterodimer and a T-cell epitope polypeptide, where the molar ratio of T-cell epitope polypeptide to E1/E2 heterodimer in the composition is from about 0.1:1 to about 25:1. For example, the molar ratio of T-cell epitope polypeptide to E1/E2 heterodimer in an immunogenic composition of the present disclosure is from about 0.1:1 to about 0.2:1, from about 0.2:1 to 0.3:1, from about 0.3:1 to about 0.4:1, from about 0.4:1 to about 0.5:1, or from about 0.5:1 to 1:1. Where multiple different T-cell epitope polypeptides are present in an immunogenic composition of the present disclosure the molar ratio of the different T-cell epitope polypeptides to E1/E2 heterodimer can each be independently from about 0.1:1 to about 0.2:1, from about 0.1:1 to about 0.5:1, from about 0.2:1 to 0.3:1, from about 0.3:1 to about 0.4:1, from about 0.4:1 to about 0.5:1, or from about 0.5:1 to 1:1. As another example, the molar ratio of any given T-cell epitope polypeptide to E1/E2 heterodimer is from 1:1 to 25:1, e.g., from 1:1 to 5:1, from 5:1 to 10:1, from 10:1 to 15:1, from 15:1 to 20:1, or from 20:1 to 25:1. In some cases, the molar ratio of a T-cell epitope polypeptide to E1/E2 heterodimer in an immunogenic composition of the present disclosure is from about 10:1 to 20:1. In some cases, the molar ratio of a T-cell epitope polypeptide to E1/E2 heterodimer in an immunogenic composition of the present disclosure is from about 10:1 to 15:1. In some cases, the molar ratio of a T-cell epitope polypeptide to E1/E2 heterodimer in a composition of the present disclosure is 15:1.

As non-limiting examples, in some cases, an immunogenic composition of the present disclosure comprises an E1/E2 heterodimer and 4 different T-cell epitope polypeptides, where the molar ratio of each T-cell epitope polypeptide to the E1/E2 heterodimer in the composition is independently from about 0.1:1 to about 25:1. As one example, in some cases, an immunogenic composition of the present disclosure comprises: a) an E1/E2 heterodimer; and b) a TP50C polypeptide (“TP50C”), a TP35-NS3 polypeptide (“TP35-NS3”), a TP27 polypeptide (“TP27”), and a TP48 polypeptide (“TP48”), where: i) the molar ratio of TP50C to E1/E2 heterodimer is from 0.1:1 to 0.5:1; ii) the molar ratio of TP35-NS3 to E1/E2 heterodimer is from 0.1:1 to 0.5:1; iii) the molar ratio of TP27 to E1/E2 heterodimer is from 0.1:1 to 0.5:1; and iv) the molar ratio of TP48 to E1/E2 heterodimer is from 0.1:1 to 0.5:1. As another example, in some cases, an immunogenic composition of the present disclosure comprises: a) an E1/E2 heterodimer; and b) a TP50C polypeptide, a TP35-NS3 polypeptide, a TP27 polypeptide, and a TP48 polypeptide, where: i) the molar ratio of TP50C to E1/E2 heterodimer is from 0.5:1 to 1:1; ii) the molar ratio of TP35-NS3 to E1/E2 heterodimer is from 0.5:1 to 1:1; iii) the molar ratio of TP27 to E1/E2 heterodimer is from 0.5:1 to 1:1; and iv) the molar ratio of TP48 to E1/E2 heterodimer is from 0.5:1 to 1:1. As another example, in some cases, an immunogenic composition of the present disclosure comprises: a) an E1/E2 heterodimer; and b) a TP50C polypeptide, a TP35-NS3 polypeptide, a TP27 polypeptide, and a TP48 polypeptide, where: i) the molar ratio of TP50C to E1/E2 heterodimer is from 1:1 to 5:1; ii) the molar ratio of TP35-NS3 to E1/E2 heterodimer is from 1:1 to 5:1; iii) the molar ratio of TP27 to E1/E2 heterodimer is from 1:1 to 5:1; and iv) the molar ratio of TP48 to E1/E2 heterodimer is from 1:1 to 5:1. As another example, in some cases, an immunogenic composition of the present disclosure comprises: a) an E1/E2 heterodimer; and b) a TP50C polypeptide, a TP35-NS3 polypeptide, a TP27 polypeptide, and a TP48 polypeptide, where: i) the molar ratio of TP50C to E1/E2 heterodimer is from 5:1 to 10:1; ii) the molar ratio of TP35-NS3 to E1/E2 heterodimer is from 5:1 to 10:1; iii) the molar ratio of TP27 to E1/E2 heterodimer is from 5:1 to 10:1; and iv) the molar ratio of TP48 to E1/E2 heterodimer is from 5:1 to 10:1. As another example, in some cases, an immunogenic composition of the present disclosure comprises: a) an E1/E2 heterodimer; and b) a TP50C polypeptide, a TP35-NS3 polypeptide, a TP27 polypeptide, and a TP48 polypeptide, where: i) the molar ratio of TP50C to E1/E2 heterodimer is from 10:1 to 25:1; ii) the molar ratio of TP35-NS3 to E1/E2 heterodimer is from 10:1 to 25:1; iii) the molar ratio of TP27 to E1/E2 heterodimer is from 10:1 to 25:1; and iv) the molar ratio of TP48 to E1/E2 heterodimer is from 10:1 to 25:1. As another example, in some cases, an immunogenic composition of the present disclosure comprises: a) an E1/E2 heterodimer; and b) a TP50C polypeptide, a TP35-NS3 polypeptide, a TP27 polypeptide, and a TP48 polypeptide, where: i) the molar ratio of TP50C to E1/E2 heterodimer is 0.1:1; ii) the molar ratio of TP35-NS3 to E1/E2 heterodimer is 0.1:1; iii) the molar ratio of TP27 to E1/E2 heterodimer is 0.1:1; and iv) the molar ratio of TP48 to E1/E2 heterodimer is 0.1:1. As another example, in some cases, an immunogenic composition of the present disclosure comprises: a) an E1/E2 heterodimer; and b) a TP50C polypeptide, a TP35-NS3 polypeptide, a TP27 polypeptide, and a TP48 polypeptide, where: i) the molar ratio of TP50C to E1/E2 heterodimer is 0.3:1; ii) the molar ratio of TP35-NS3 to E1/E2 heterodimer is 0.3:1; iii) the molar ratio of TP27 to E1/E2 heterodimer is 0.3:1; and iv) the molar ratio of TP48 to E1/E2 heterodimer is 0.3:1. As another example, in some cases, an immunogenic composition of the present disclosure comprises: a) an E1/E2 heterodimer; and b) a TP50C polypeptide, a TP35-NS3 polypeptide, a TP27 polypeptide, and a TP48 polypeptide, where: i) the molar ratio of TP50C to E1/E2 heterodimer is 0.5:1; ii) the molar ratio of TP35-NS3 to E1/E2 heterodimer is 0.5:1; iii) the molar ratio of TP27 to E1/E2 heterodimer is 0.5:1; and iv) the molar ratio of TP48 to E1/E2 heterodimer is 0.5:1. As another example, in some cases, an immunogenic composition of the present disclosure comprises: a) an E1/E2 heterodimer; and b) a TP50C polypeptide, a TP35-NS3 polypeptide, a TP27 polypeptide, and a TP48 polypeptide, where: i) the molar ratio of TP50C to E1/E2 heterodimer is 1:1; ii) the molar ratio of TP35-NS3 to E1/E2 heterodimer is 1:1; iii) the molar ratio of TP27 to E1/E2 heterodimer is 1:1; and iv) the molar ratio of TP48 to E1/E2 heterodimer is 1:1. As another example, in some cases, an immunogenic composition of the present disclosure comprises: a) an E1/E2 heterodimer; and b) a TP50C polypeptide, a TP35-NS3 polypeptide, a TP27 polypeptide, and a TP48 polypeptide, where: i) the molar ratio of TP50C to E1/E2 heterodimer is 3:1; ii) the molar ratio of TP35-NS3 to E1/E2 heterodimer is 3:1; iii) the molar ratio of TP27 to E1/E2 heterodimer is 3:1; and iv) the molar ratio of TP48 to E1/E2 heterodimer is 3:1. As another example, in some cases, an immunogenic composition of the present disclosure comprises: a) an E1/E2 heterodimer; and b) a TP50C polypeptide, a TP35-NS3 polypeptide, a TP27 polypeptide, and a TP48 polypeptide, where: i) the molar ratio of TP50C to E1/E2 heterodimer is 9:1; ii) the molar ratio of TP35-NS3 to E1/E2 heterodimer is 9:1; iii) the molar ratio of TP27 to E1/E2 heterodimer is 9:1; and iv) the molar ratio of TP48 to E1/E2 heterodimer is 9:1. As another example, in some cases, an immunogenic composition of the present disclosure comprises: i) an E1/E2 heterodimer; and ii) 4 different T-cell epitope polypeptides, where the 4 different T-cell epitope polypeptides are a TP50C polypeptide, a TP35-NS3 polypeptide, a TP27 polypeptide, and a TP48 polypeptide. As one example, in some cases, an immunogenic composition of the present disclosure comprises: a) an E1/E2 heterodimer; and b) a TP50C polypeptide, a TP35-NS3 polypeptide, a TP27 polypeptide, and a TP48 polypeptide, where: i) the molar ratio of TP50C to E1/E2 heterodimer is 3:1; ii) the molar ratio of TP35-NS3 to E1/E2 heterodimer is 3:1; iii) the molar ratio of TP27 to E1/E2 heterodimer is 3:1; and iv) the molar ratio of TP48 to E1/E2 heterodimer is 9:1. Amino acid sequences of TP50C, TP35-NS3, TP27, and TP48 polypeptides are provided above. In some cases, the TP50C polypeptide comprises the following amino acid sequence: GVYLLPRRGPRLGVRATRKTSERSQPRGRRQPIPKARRSEGRSWAQPGYP (SEQ ID NO:87) and has a length of 50 amino acids; the TP35-NS3 polypeptide comprises the following amino acid sequence: KSTKVPAAYAAQGYKVLVLNPSVAATLGFGAYMSK (SEQ ID NO:79) and has a length of 35 amino acids; the TP27 polypeptide comprises the following amino acid sequence: LEQIKGGRHLIFCHSKKKCDELAAKLR (SEQ ID NO:103) and has a length of 27 amino acids; and the TP48 polypeptide comprises the following amino acid sequence: ILRRHVGPGEGAVQWMNRLIAFASRGNHVSPTHYVPESDAAARVTQIL (SEQ ID NO:136) and has a length of 48 amino acids.

As non-limiting examples, in some cases, an immunogenic composition of the present disclosure comprises an E1/E2 heterodimer and 2 different T-cell epitope polypeptides, where the molar ratio of each T-cell epitope polypeptide to the E1/E2 heterodimer in the composition is independently from about 0.1:1 to about 25:1. As one example, in some cases, an immunogenic composition of the present disclosure comprises: a) an E1/E2 heterodimer; and b) a TP465 polypeptide (“TP465”) and a TP48 polypeptide (“TP48”), where: i) the molar ratio of TP465 to E1/E2 heterodimer is from 0.1:1 to 0.5:1; and ii) the molar ratio of TP48 to E1/E2 heterodimer is from 0.1:1 to 0.5:1. As another example, in some cases, an immunogenic composition of the present disclosure comprises: a) an E1/E2 heterodimer; and b) a TP465 polypeptide and a TP48 polypeptide, where: i) the molar ratio of TP465 to E1/E2 heterodimer is from 0.5:1 to 1:1; and ii) the molar ratio of TP48 to E1/E2 heterodimer is from 0.5:1 to 1:1. As another example, in some cases, an immunogenic composition of the present disclosure comprises: a) an E1/E2 heterodimer; and b) a TP465 polypeptide and a TP48 polypeptide, where: i) the molar ratio of TP465 to E1/E2 heterodimer is from 1:1 to 5:1; and ii) the molar ratio of TP48 to E1/E2 heterodimer is from 1:1 to 5:1. As another example, in some cases, an immunogenic composition of the present disclosure comprises: a) an E1/E2 heterodimer; and b) a TP465 polypeptide and a TP48 polypeptide, where: i) the molar ratio of TP465 to E1/E2 heterodimer is from 5:1 to 10:1; and ii) the molar ratio of TP48 to E1/E2 heterodimer is from 5:1 to 10:1. As another example, in some cases, an immunogenic composition of the present disclosure comprises: a) an E1/E2 heterodimer; and b) a TP465 polypeptide and a TP48 polypeptide, where: i) the molar ratio of TP465 to E1/E2 heterodimer is from 10:1 to 25:1; and ii) the molar ratio of TP48 to E1/E2 heterodimer is from 10:1 to 25:1. As another example, in some cases, an immunogenic composition of the present disclosure comprises: a) an E1/E2 heterodimer; and b) a TP465 polypeptide and a TP48 polypeptide, where: i) the molar ratio of TP465 to E1/E2 heterodimer is 1:1; and ii) the molar ratio of TP48 to E1/E2 heterodimer is 1:1. As another example, in some cases, an immunogenic composition of the present disclosure comprises: a) an E1/E2 heterodimer; and b) a TP465 polypeptide and a TP48 polypeptide, where: i) the molar ratio of TP465 to E1/E2 heterodimer is 3:1; and ii) the molar ratio of TP48 to E1/E2 heterodimer is 3:1. As another example, in some cases, an immunogenic composition of the present disclosure comprises: a) an E1/E2 heterodimer; and b) a TP465 polypeptide and a TP48 polypeptide, where: i) the molar ratio of TP465 to E1/E2 heterodimer is 9:1; and ii) the molar ratio of TP48 to E1/E2 heterodimer is 9:1. As another example, in some cases, an immunogenic composition of the present disclosure comprises: a) an E1/E2 heterodimer; and b) a TP465 polypeptide and a TP48 polypeptide, where: i) the molar ratio of TP465 to E1/E2 heterodimer is 0.1:1; and ii) the molar ratio of TP48 to E1/E2 heterodimer is 0.1:1. As another example, in some cases, an immunogenic composition of the present disclosure comprises: a) an E1/E2 heterodimer; and b) a TP465 polypeptide and a TP48 polypeptide, where: i) the molar ratio of TP465 to E1/E2 heterodimer is 0.3:1; and ii) the molar ratio of TP48 to E1/E2 heterodimer is 0.3:1. As another example, in some cases, an immunogenic composition of the present disclosure comprises: a) an E1/E2 heterodimer; and b) a TP465 polypeptide and a TP48 polypeptide, where: i) the molar ratio of TP465 to E1/E2 heterodimer is 0.5:1; and ii) the molar ratio of TP48 to E1/E2 heterodimer is 0.5:1. As another example, in some cases, an immunogenic composition of the present disclosure comprises: a) an E1/E2 heterodimer; and b) a TP465 polypeptide and a TP48 polypeptide, where: i) the molar ratio of TP465 to E1/E2 heterodimer is 3:1; and ii) the molar ratio of TP48 to E1/E2 heterodimer is 9:1. As another example, in some cases, an immunogenic composition of the present disclosure comprises: a) an E1/E2 heterodimer; and b) a TP465 polypeptide and a TP48 polypeptide, where: i) the molar ratio of TP465 to E1/E2 heterodimer is 9:1; and ii) the molar ratio of TP48 to E1/E2 heterodimer is 3:1. Amino acid sequences of TP465 polypeptides and TP48 polypeptides are provided above.

As non-limiting examples, in some cases, an immunogenic composition of the present disclosure comprises an E1/E2 heterodimer and 2 different T-cell epitope polypeptides, where the molar ratio of each T-cell epitope polypeptide to the E1/E2 heterodimer in the composition is independently from about 0.1:1 to about 25:1. As one example, in some cases, an immunogenic composition of the present disclosure comprises: a) an E1/E2 heterodimer; and b) a TP465 polypeptide (“TP465”) and a TP65 polypeptide (“TP65”), where: i) the molar ratio of TP465 to E1/E2 heterodimer is from 0.1:1 to 0.5:1; and ii) the molar ratio of TP65 to E1/E2 heterodimer is from 0.1:1 to 0.5:1. As another example, in some cases, an immunogenic composition of the present disclosure comprises: a) an E1/E2 heterodimer; and b) a TP465 polypeptide and a TP65 polypeptide, where: i) the molar ratio of TP465 to E1/E2 heterodimer is from 0.5:1 to 1:1; and ii) the molar ratio of TP65 to E1/E2 heterodimer is from 0.5:1 to 1:1. As another example, in some cases, an immunogenic composition of the present disclosure comprises: a) an E1/E2 heterodimer; and b) a TP465 polypeptide and a TP65 polypeptide, where: i) the molar ratio of TP465 to E1/E2 heterodimer is from 1:1 to 5:1; and ii) the molar ratio of TP65 to E1/E2 heterodimer is from 1:1 to 5:1. As another example, in some cases, an immunogenic composition of the present disclosure comprises: a) an E1/E2 heterodimer; and b) a TP465 polypeptide and a TP65 polypeptide, where: i) the molar ratio of TP465 to E1/E2 heterodimer is from 5:1 to 10:1; and ii) the molar ratio of TP65 to E1/E2 heterodimer is from 5:1 to 10:1. As another example, in some cases, an immunogenic composition of the present disclosure comprises: a) an E1/E2 heterodimer; and b) a TP465 polypeptide and a TP65 polypeptide, where: i) the molar ratio of TP465 to E1/E2 heterodimer is from 10:1 to 25:1; and ii) the molar ratio of TP65 to E1/E2 heterodimer is from 10:1 to 25:1. As another example, in some cases, an immunogenic composition of the present disclosure comprises: a) an E1/E2 heterodimer; and b) a TP465 polypeptide and a TP65 polypeptide, where: i) the molar ratio of TP465 to E1/E2 heterodimer is 1:1; and ii) the molar ratio of TP65 to E1/E2 heterodimer is 1:1. As another example, in some cases, an immunogenic composition of the present disclosure comprises: a) an E1/E2 heterodimer; and b) a TP465 polypeptide and a TP65 polypeptide, where: i) the molar ratio of TP465 to E1/E2 heterodimer is 3:1; and ii) the molar ratio of TP65 to E1/E2 heterodimer is 3:1. As another example, in some cases, an immunogenic composition of the present disclosure comprises: a) an E1/E2 heterodimer; and b) a TP465 polypeptide and a TP65 polypeptide, where: i) the molar ratio of TP465 to E1/E2 heterodimer is 9:1; and ii) the molar ratio of TP65 to E1/E2 heterodimer is 9:1. As another example, in some cases, an immunogenic composition of the present disclosure comprises: a) an E1/E2 heterodimer; and b) a TP465 polypeptide and a TP65 polypeptide, where: i) the molar ratio of TP465 to E1/E2 heterodimer is 0.1:1; and ii) the molar ratio of TP65 to E1/E2 heterodimer is 0.1:1. As another example, in some cases, an immunogenic composition of the present disclosure comprises: a) an E1/E2 heterodimer; and b) a TP465 polypeptide and a TP65 polypeptide, where: i) the molar ratio of TP465 to E1/E2 heterodimer is 0.3:1; and ii) the molar ratio of TP65 to E1/E2 heterodimer is 0.3:1. As another example, in some cases, an immunogenic composition of the present disclosure comprises: a) an E1/E2 heterodimer; and b) a TP465 polypeptide and a TP65 polypeptide, where: i) the molar ratio of TP465 to E1/E2 heterodimer is 0.5:1; and ii) the molar ratio of TP65 to E1/E2 heterodimer is 0.5:1. As another example, in some cases, an immunogenic composition of the present disclosure comprises: a) an E1/E2 heterodimer; and b) a TP465 polypeptide and a TP65 polypeptide, where: i) the molar ratio of TP465 to E1/E2 heterodimer is 3:1; and ii) the molar ratio of TP65 to E1/E2 heterodimer is 9:1. As another example, in some cases, an immunogenic composition of the present disclosure comprises: a) an E1/E2 heterodimer; and b) a TP465 polypeptide and a TP65 polypeptide, where: i) the molar ratio of TP465 to E1/E2 heterodimer is 9:1; and ii) the molar ratio of TP65 to E1/E2 heterodimer is 3:1. Amino acid sequences of TP465 polypeptides and TP65 polypeptides are provided above.

As non-limiting examples, in some cases, an immunogenic composition of the present disclosure comprises an E1/E2 heterodimer and 2 different T-cell epitope polypeptides, where the molar ratio of each T-cell epitope polypeptide to the E1/E2 heterodimer in the composition is independently from about 0.1:1 to about 25:1. As one example, in some cases, an immunogenic composition of the present disclosure comprises: a) an E1/E2 heterodimer; and b) a TP465 polypeptide (“TP465”) and a TP156 polypeptide (“TP156”), where: i) the molar ratio of TP465 to E1/E2 heterodimer is from 0.1:1 to 0.5:1; and ii) the molar ratio of TP156 to E1/E2 heterodimer is from 0.1:1 to 0.5:1. As another example, in some cases, an immunogenic composition of the present disclosure comprises: a) an E1/E2 heterodimer; and b) a TP465 polypeptide and a TP156 polypeptide, where: i) the molar ratio of TP465 to E1/E2 heterodimer is from 0.5:1 to 1:1; and ii) the molar ratio of TP156 to E1/E2 heterodimer is from 0.5:1 to 1:1. As another example, in some cases, an immunogenic composition of the present disclosure comprises: a) an E1/E2 heterodimer; and b) a TP465 polypeptide and a TP156 polypeptide, where: i) the molar ratio of TP465 to E1/E2 heterodimer is from 1:1 to 5:1; and ii) the molar ratio of TP156 to E1/E2 heterodimer is from 1:1 to 5:1. As another example, in some cases, an immunogenic composition of the present disclosure comprises: a) an E1/E2 heterodimer; and b) a TP465 polypeptide and a TP156 polypeptide, where: i) the molar ratio of TP465 to E1/E2 heterodimer is from 5:1 to 10:1; and ii) the molar ratio of TP156 to E1/E2 heterodimer is from 5:1 to 10:1. As another example, in some cases, an immunogenic composition of the present disclosure comprises: a) an E1/E2 heterodimer; and b) a TP465 polypeptide and a TP156 polypeptide, where: i) the molar ratio of TP465 to E1/E2 heterodimer is from 10:1 to 25:1; and ii) the molar ratio of TP156 to E1/E2 heterodimer is from 10:1 to 25:1. As another example, in some cases, an immunogenic composition of the present disclosure comprises: a) an E1/E2 heterodimer; and b) a TP465 polypeptide and a TP156 polypeptide, where: i) the molar ratio of TP465 to E1/E2 heterodimer is 1:1; and ii) the molar ratio of TP156 to E1/E2 heterodimer is 1:1. As another example, in some cases, an immunogenic composition of the present disclosure comprises: a) an E1/E2 heterodimer; and b) a TP465 polypeptide and a TP156 polypeptide, where: i) the molar ratio of TP465 to E1/E2 heterodimer is 3:1; and ii) the molar ratio of TP156 to E1/E2 heterodimer is 3:1. As another example, in some cases, an immunogenic composition of the present disclosure comprises: a) an E1/E2 heterodimer; and b) a TP465 polypeptide and a TP156 polypeptide, where: i) the molar ratio of TP465 to E1/E2 heterodimer is 9:1; and ii) the molar ratio of TP156 to E1/E2 heterodimer is 9:1. As another example, in some cases, an immunogenic composition of the present disclosure comprises: a) an E1/E2 heterodimer; and b) a TP465 polypeptide and a TP156 polypeptide, where: i) the molar ratio of TP465 to E1/E2 heterodimer is 0.1:1; and ii) the molar ratio of TP156 to E1/E2 heterodimer is 0.1:1. As another example, in some cases, an immunogenic composition of the present disclosure comprises: a) an E1/E2 heterodimer; and b) a TP465 polypeptide and a TP156 polypeptide, where: i) the molar ratio of TP465 to E1/E2 heterodimer is 0.3:1; and ii) the molar ratio of TP156 to E1/E2 heterodimer is 0.3:1. As another example, in some cases, an immunogenic composition of the present disclosure comprises: a) an E1/E2 heterodimer; and b) a TP465 polypeptide and a TP156 polypeptide, where: i) the molar ratio of TP465 to E1/E2 heterodimer is 0.5:1; and ii) the molar ratio of TP156 to E1/E2 heterodimer is 0.5:1. As another example, in some cases, an immunogenic composition of the present disclosure comprises: a) an E1/E2 heterodimer; and b) a TP465 polypeptide and a TP156 polypeptide, where: i) the molar ratio of TP465 to E1/E2 heterodimer is 3:1; and ii) the molar ratio of TP156 to E1/E2 heterodimer is 9:1. As another example, in some cases, an immunogenic composition of the present disclosure comprises: a) an E1/E2 heterodimer; and b) a TP465 polypeptide and a TP156 polypeptide, where: i) the molar ratio of TP465 to E1/E2 heterodimer is 9:1; and ii) the molar ratio of TP156 to E1/E2 heterodimer is 3:1. Amino acid sequences of TP465 polypeptides and TP156 polypeptides are provided above.

The initial administration can be followed by booster immunization of the same immunogenic composition or a different immunogenic composition. In some instances, a subject method of inducing an immune response involves an initial administration of an immunogenic composition of the present disclosure, followed by at least one booster, and in some instances involves two or more (e.g., three, four, or five) boosters. The interval between an initial administration and a booster, or between a give booster and a subsequent booster, can be from about 1 week to about 12 weeks, e.g., from about 1 week to about 2 weeks, from about 2 weeks to about 4 weeks, from about 4 weeks to about 6 weeks, from about 6 weeks to about 8 weeks, from about 8 weeks to about 10 weeks, or from about 10 weeks to about 12 weeks. The interval between an initial administration and a booster, or between a give booster and a subsequent booster, can be from 4 months to 6 months, or from 6 months to 1 year.

In some cases, a first dose of an immunogenic composition of the present disclosure is administered at a first time, where the administration is intramuscular; a second dose of an immunogenic composition of the present disclosure is administered at a second time, where the administration is intranasal administration; a third dose of an immunogenic composition of the present disclosure is administered at a third time, where the administration is intranasal administration. In some cases, the second dose (a “first booster”) is administered from about 1 week to about 12 weeks, e.g., from about 1 week to about 2 weeks, from about 2 weeks to about 4 weeks, from about 4 weeks to about 6 weeks, from about 6 weeks to about 8 weeks, from about 8 weeks to about 10 weeks, from about 10 weeks to about 12 weeks, from 4 months to 6 months, or from 6 months to 1 year, after the first dose. In some cases, the third dose (a “second booster”) is administered from about 1 week to about 12 weeks, e.g., from about 1 week to about 2 weeks, from about 2 weeks to about 4 weeks, from about 4 weeks to about 6 weeks, from about 6 weeks to about 8 weeks, from about 8 weeks to about 10 weeks, from about 10 weeks to about 12 weeks, from 4 months to 6 months, or from 6 months to 1 year, after the second dose.

In general, immunization can be accomplished by administration of an immunogenic composition of the present disclosure by any suitable route, including administration of the composition orally, nasally (e.g., intranasally), nasopharyngeally, parenterally, enterically, gastrically, topically, transdermally, subcutaneously, intramuscularly, or intradermally. In some cases, an immunogenic composition of the present disclosure is administered intramuscularly. In some cases, an immunogenic composition of the present disclosure is administered subcutaneously. Immunization can be accomplished by administration of an immunogenic composition of the present disclosure in tablet, solid, powdered, liquid, or aerosol form, locally or systemically, with or without added excipients. An immunogenic composition of the present disclosure is in many cases in liquid form. Actual methods for preparing parenterally administrable compositions will be known or apparent to those skilled in the art and are described in more detail in such publications as Remington's Pharmaceutical Science, 15th ed., Mack Publishing Company, Easton, Pa. (1980). In some instances, immunization is accomplished by intramuscular injection of an immunogenic composition of the present disclosure.

### Individuals Suitable for Administration

Individuals who are suitable for administration with an immunogenic composition of the present disclosure include immunologically naïve individuals (e.g., individuals who have not been infected with HCV and/or who have not been administered with an HCV vaccine). Individuals suitable for administration include humans.

Individuals who are suitable for administration with an immunogenic composition of the present disclosure composition of the present disclosure include individuals who are at greater risk than the general population of becoming infected with HCV, where such individuals include, e.g., intravenous drug users; individuals who are the recipients, or the prospective recipients, of blood or blood products from another (donor) individual(s); individuals who are the recipients, or the prospective recipients, of non-autologous cells, tissues, or organs from another (donor) individual; health care workers; emergency medical and non-medical personnel (e.g., first responders; fire fighters; emergency medical team personnel; etc.) and the like.

Individuals who are suitable for administration with an immunogenic composition of the present disclosure composition of the present disclosure include individuals who recently became exposed to HCV or who recently became infected with HCV. For example, a subject immunogenic composition can be administered to an individual within from about 24 hours to about 48 hours, from about 48 hours to about 1 week, or from about 1 week to about 4 weeks, following possible or suspected exposure to HCV or following infection with HCV.

Individuals who are suitable for administration with an immunogenic composition of the present disclosure composition of the present disclosure include individuals who were previously infected with HCV, who were treated for HCV, and who were cured.

Individuals who are suitable for administration with an immunogenic composition of the present disclosure composition of the present disclosure include individuals who have been diagnosed as having an HCV infection, and include chronically infected individuals. In some cases, an individual who has been diagnosed as having an HCV infection is treated with an anti-viral agent and an immunogenic composition of the present disclosure. Suitable anti-viral agents for treating HCV infection include, e.g., ribavirin (1-β-D-ribofuranosyl-1H-1,2,4-triazole-3-carboxamide); interferon-alpha (IFN-α) (where “IFN-α” includes IFN-α2a; IFN-α2b; IFN-α that is conjugated with poly(ethylene glycol) (“pegylated IFN-α), where the pegylated IFN-α can be pegylated IFN-α2a or pegylated IFN-α 2b); an HCV NS3 protease inhibitor (e.g., boceprevir; telaprevir); and an HCV NS5 protease inhibitor.

In some cases, an individual who has been diagnosed as having an HCV infection is treated with, e.g.: 1) IFN-α+ribavirin; and an immunogenic composition of the present disclosure; 2) IFN-α+ribavirin+an HCV protease inhibitor (e.g., boceprevir or telaprevir); and an immunogenic composition of the present disclosure; 3) Harvoni; and an immunogenic composition of the present disclosure; 4) an inhibitor of HCV NS5B; and an immunogenic composition of the present disclosure; 5) an inhibitor of HCV NS5A; and an immunogenic composition of the present disclosure; or 6) an inhibitor of HCV NS5B+an inhibitor of HCV NS5A; and an immunogenic composition of the present disclosure. Suitable anti-viral agents for treating HCV infection include Sovaldi (Sofosbuvir; a nucleotide analog that functions as an NS5B polymerase inhibitor), alone or in combination with pegylated IFN-α and ribavirin; and Harvoni. Harvoni is a formulation comprising 90 mg ledipasvir and 400 mg sofosbuvir. Ledipasvir is an inhibitor of HCV NS5A.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. An immunogenic composition comprising:
a1) one or more T-cell epitope polypeptides, or a fusion polypeptide comprising two or more T-cell epitope polypeptides, wherein the T-cell epitope polypeptides are selected from:
   i) a TP35-NS3 T-cell epitope polypeptide comprising the amino acid sequence:

(SEQ ID NO: 73)
KSTKVPX₁AYX₂X₃QGYX₄VLVLNPSVAATLGFGX₅X₆X₇SX₈,

wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of 35 amino acids;
ii) a TP50C T-cell epitope polypeptide comprising the amino acid sequence:
GVYX₁LPRRGPRLGVRX₂TRKX₃SERSQPRGRRQX₄IPKX₅X₆X₇X₈X₉GX₁₀X₁₁WX₁₂ X₁₃PGYP (SEQ ID NO:80), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of 50 amino acids;
   iii) a TP27 T-cell epitope polypeptide comprising the amino acid sequence: X₁X₂X₃X₄KGGRHLIFCHSKKKCDEX₅AX₆X₇LX₈(SEQ ID NO:94), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of 27 amino acids; and
   iv) a TP48 T-cell epitope polypeptide comprising the amino acid sequence: X₁X₂X₃RHX₄GX₅X₆EGX₇X₈QWMNRLIAFASRGNHVX₉PTHYX₁₀X₁₁X₁₂X₃DAX₁₄X₅X₁₆VX₁₇X₁₈X₁₉ L (SEQ ID NO:134), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein the TP48 T-cell epitope polypeptide has a length of 48 amino acids; and
b1) a second-generation lipid adjuvant (SLA) that is a toll-like receptor 4 (TLR4) agonist formulated in: i) a stable emulsion; or ii) a liposomal composition comprising a saponin; or
a2) one or more T-cell epitope polypeptides, or a fusion polypeptide comprising two or more T-cell epitope polypeptides, wherein the T-cell epitope polypeptides are selected from:
   i) a TP65 polypeptide comprising the amino acid sequence: TPIDTTIMAKNEVFCVDPX₆KGGRKPARLIVYPDLGVRVCEKMALYDVVQKLPQAVMGX₇SYGF QYS (SEQ ID NO:152), where X₆ is E, T, or V, and X₇ is S, A, or P, wherein the TP65 polypeptide has a length of 65 amino acids;
   ii) a TP50C T-cell epitope polypeptide comprising the amino acid sequence:
GVYX₁LPRRGPRLGVRX₂TRKX₃SERSQPRGRRQX₄IPKX₅X₆X₇X₈X₉GX₁₀X₁₁WX₁₂ X₁₃PGYP (SEQ ID NO:80), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of 50 amino acids;
iii) a TP240 T-cell epitope polypeptide comprising the amino acid sequence:

(SEQ ID NO: 156)
SYQVGYLHAPTGSGKSTKVPAAYAAQGYKVLVLNPSVAATLGFGAYMSK
AHGIDPNIRTGVRTVTTGAPITYSTYGKFLADGGCSGGAYDIIICDECH
SX₁DATTILGIGTVLDQAETAGARLVVLATATPPGSVTVPHPNIEEVAL
GX₂EGEIPFYGKAIPLX₃X₄IKGGRHLIFCHSKKKCDELAAKLRGMGLNA
VAYYRGLDVSVIPTX₅GDVVVVATDALMTGYTGDFDSVIDCNVAVTQT,

where X₁ is T, V, or Q, X₂ is T, N, Q, H, or S, X₃ is E, S, or A, X₄ is V, T, Y, F, or L, and X₅ is S, Q, or T, wherein the TP240 polypeptide has a length of 240 amino acids; and
iv) a TP48 T-cell epitope polypeptide comprising the amino acid sequence: X₁X₂X₃RHX₄GX₅X₆EGX₇X₈QWMNRLIAFASRGNHVX₉PTHYX₁₀X₁ X₁₂X₃DAX₁₄X₅X₁₆VX₁₇X₁₈X₁₉ L (SEQ ID NO:134), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein the TP48 T-cell epitope polypeptide has a length of 48 amino acids; and
b2) a second-generation lipid adjuvant (SLA) that is a toll-like receptor 4 (TLR4) agonist formulated in: i) a stable emulsion; or ii) a liposomal composition comprising a saponin;
or
a3) one or more T-cell epitope polypeptides, or a fusion polypeptide comprising two or more T-cell epitope polypeptides, wherein the T-cell epitope polypeptides are selected from:
i) a TP35-NS3 T-cell epitope polypeptide comprising the amino acid sequence: KSTKVPX₁AYX₂X₃QGYX₄VLVLNPSVAATLGFGX₅X₆X₇SX₈ (SEQ ID NO:73), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of 35 amino acids;
   ii) a TP50C T-cell epitope polypeptide comprising the amino acid sequence: GVYX₁LPRRGPRLGVRX₂TRKX₃SERSQPRGRRQX₄IPKX₅XX₇XX₉GX₁₀X₁WX₁₂X₁₃PGYP (SEQ ID NO:80), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of 50 amino acids;
   iii) a TP27 T-cell epitope polypeptide comprising the amino acid sequence: X₁X₂X₃X₄KGGRHLIFCHSKKKCDEX₅AX₆X₇LX₈(SEQ ID NO:94), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of 27 amino acids; and
   iv) a TP48 T-cell epitope polypeptide comprising the amino acid sequence: X₁X₂X₃RHX₄GX₅X₆EGX₇X₈QWMNRLIAFASRGNHVX₉PTHYX₁₀X₁ X₁₂X₃DAX₁₄X₅X₁₆VX₁₇X₁₈X₁₉ L (SEQ ID NO:134), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein the TP48 T-cell epitope polypeptide has a length of 48 amino acids;
   v) a TP240 T-cell epitope polypeptide comprising an amino acid sequence having at least 50%, at least 60%, at least 70%, at least 80%, at least 90%, or at least 95%, amino acid sequence identity to the sequence:

(SEQ ID NO: 157)
SYQVGYLHAPTGSGKSTKVPAAYAAQGYKVLVLNPSVAATLGFGAYMSK
AHGIDPNIRTGVRTVTTGAPITYSTYGKFLADGGCSGGAYDIIICDECH
STDATTILGIGTVLDQAETAGARLVVLATATPPGSVTVPHPNIEEVALG
TEGEIPFYGKAIPLEVIKGGRHLIFCHSKKKCDELAAKLRGMGLNAVAY
YRGLDVSVIPTSGDVVVVATDALMTGYTGDFDSVIDCNVAVTQT,

wherein the TP240 T-cell epitope polypeptide has a length of 240 amino acids;
vi) a TP65 T-cell epitope polypeptide comprising an amino acid sequence having at least 50%, at least 60%, at least 70%, at least 80%, at least 90%, or at least 95%, amino acid sequence identity to the sequence: TPIDTTIMAKNEVFCVDPEKGGRKPARLIVYPDLGVRVCEKMALYDVVQKLPQAVMGSSYGFQ YS (SEQ ID NO:153), wherein the TP65 T-cell epitope polypeptide has a length of 65 amino acids;
vii) a TP156 T-cell epitope polypeptide comprising an amino acid sequence having at least 50%, at least 60%, at least 70%, at least 80%, at least 90%, or at least 95%, amino acid sequence identity to the sequence: KFPGGGQIVGGVYLLPRRGPRLGVRATRKTSERSQPRGRRQPIPKARRPEGRTWAQPGYPWPLY GNEGCGWAGWLLSPRGSRPSWGPTDPRRRSRNLGKVIDTLTCGFADLMGYIPLVGAPLGGAAR ALAHGVRVLEDGVNYATGNLPGCSFSIFL (SEQ ID NO:160), wherein the TP156 T-cell epitope polypeptide has a length of 156 amino acids;
viii) a TP465 T-cell epitope polypeptide comprising an amino acid sequence having at least 50%, at least 60%, at least 70%, at least 80%, at least 90%, or at least 95%, amino acid sequence identity to the amino acid sequence depicted in any one of FIG. 11A-11C, wherein the TP465 T-cell epitope polypeptide has a length of 465 amino acids;
ix) a TP23 T-cell epitope polypeptide comprising the amino acid sequence: DVVVX₁X₂TDALMTGX₃TGDFDSVID (SEQ ID NO:89), wherein X₁ is V or C; X₂ is A or S; and X₃ is F or Y; wherein the TP23 polypeptide has a length of 23 amino acids;
x) a TP35-NS4 T-cell epitope polypeptide comprising the amino acid sequence: X₁X₂X₃RHX₄GX₅X₆EGX₇X₈QWMNRLIAFASRGNHVX₉PTHYX₁₀ (SEQ ID NO:105), where X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; and X₁₀ is V or I; wherein the TP35-NS4 polypeptide has a length of 35 amino acids;
xi) a TP42 T-cell epitope polypeptide comprising the amino acid sequence:

(SEQ ID NO: 112)
X₁X₂X₃GEIPFYGX₄AIPX₅X₆X₇X₈KGGRHLIFCHSKKKCDEX₉AX₁₀X₁₁
LX₁₂X₁₃,

where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S; wherein the TP42 polypeptide has a length of 42 amino acids;
xii) a TP45 T-cell epitope polypeptide comprising the amino acid sequence: X₁X₂AVAX₃YRGX₄DVX₅X₆IPX₇X₈GDVVVX₉X₁₀TDALMTGX₁₁TGDFDSVIDX₁₂X₁₃ X₁₄ (SEQ ID NO:129), where X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K; and wherein the TP45 T-cell epitope polypeptide has a length of 45 amino acids;
xiii) a TP33 T-cell epitope polypeptide comprising the amino acid sequence: HSKKKCDELAX₁X₂LX₃X₄X₅GX₆NAVAYYRGLDVSX₇IP (SEQ ID NO:143), where X₁ is A or S; X₂ is K or A; X₃ is V, S, R, or T; X₄ is A or G; X₅ is L or M; X₆ is I, L, or V; and X₇ is V or I; wherein the TP33 polypeptide has a length of 33 amino acids;
xiv) a TP42-2 polypeptide comprising the amino acid sequence: KGGRHLIFCHSKKKCDELAX₁X₂LX₃X₄X₅GX₆NAVAYYRGLDVSX₇IP (SEQ ID NO:148), where X₁ is A or S; X₂ is K or A; X₃ is V, S, R, or T; X₄ is A or G; X₅ is L or M; X₆ is I, L, or V; and X₇ is V or I; wherein the T42-2 polypeptide has a length of 42 amino acids;
xv) a TP29 polypeptide comprising an amino acid sequence having at least 50% amino acid sequence identity to the TP29 amino acid sequence depicted in FIG. 12A-12E, wherein the TP29 polypeptide has a length of 29 amino acids;
xvi) a TP50-NS2 polypeptide comprising an amino acid sequence having at least 50% amino acid sequence identity to the TP50-NS2 amino acid sequence depicted in FIG. 12A-12E, wherein the TP50-NS2 polypeptide has a length of 50 amino acids;
xvii) a TP52 polypeptide comprising an amino acid sequence having at least 50% amino acid sequence identity to the TP25 amino acid sequence depicted in FIG. 12A-12E, wherein the TP52 polypeptide has a length of 52 amino acids;
xviii) a TP70 polypeptide comprising an amino acid sequence having at least 50% amino acid sequence identity to the TP70 amino acid sequence depicted in FIG. 12A-12E, wherein the TP70 polypeptide has a length of 70 amino acids;
xix) a TP100 polypeptide comprising an amino acid sequence having at least 50% amino acid sequence identity to the TP100 amino acid sequence depicted in FIG. 12A-12E, wherein the TP100 polypeptide has a length of 100 amino acids;
xx) a TP171 polypeptide comprising an amino acid sequence having at least 50% amino acid sequence identity to the TP171 amino acid sequence depicted in FIG. 12A-12E, wherein the TP171 polypeptide has a length of 171 amino acids;
xxi) a TP228 polypeptide comprising an amino acid sequence having at least 50% amino acid sequence identity to the TP228 amino acid sequence depicted in FIG. 12A-12E, wherein the TP228 polypeptide has a length of 228 amino acids;
xxii) a TP553 polypeptide comprising an amino acid sequence having at least 50% amino acid sequence identity to the TP553 amino acid sequence depicted in FIG. 12A-12E, wherein the TP553 polypeptide has a length of 553 amino acids
xxiii) a TP778 polypeptide comprising an amino acid sequence having at least 50% amino acid sequence identity to the TP778 amino acid sequence depicted in FIG. 12A-12E, wherein the TP778 polypeptide has a length of 778 amino acids; and
xxiv) a TP1987 polypeptide comprising an amino acid sequence having at least 50% amino acid sequence identity to the TP1987 amino acid sequence depicted in FIG. 12A-12E, wherein the TP1987 polypeptide has a length of 1987 amino acids; and
b3) a second-generation lipid adjuvant (SLA) that is a toll-like receptor 4 (TLR4) agonist formulated in: i) a stable emulsion; or ii) a liposomal composition comprising a saponin.

Aspect 2. The immunogenic composition of aspect 1, wherein the SLA has the following structure:

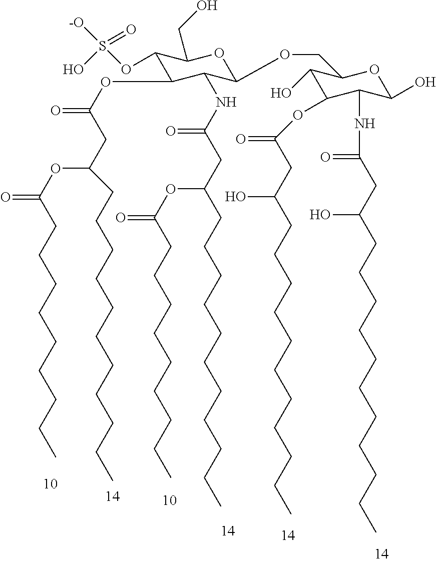

Aspect 3. The immunogenic composition of aspect 1 or aspect 2, wherein the liposomal composition comprising a saponin comprises a sterol.

Aspect 4. The immunogenic composition of aspect 3, wherein the saponin is complexed to the sterol.

Aspect 5. The immunogenic composition of aspect 3 or aspect 4, wherein the sterol is cholesterol.

Aspect 6. The immunogenic composition of any one of aspects 1-5, wherein the liposomal composition comprises a phospholipid.

Aspect 7. The immunogenic composition of aspect 6, wherein the phospholipid is selected from the group consisting of DLPC, DMPC, DPPC, DSPC, DOPC, POPC, DLPG, DMPG, DPPG, DSPG, DOPG, DSTAP, DPTAP, DSPE, DPPE, DMPE, and DLPE.

Aspect 8. The immunogenic composition of any one of aspects 1-7, wherein the saponin is QS21, QS17, QS7, synthetic QS21 (SQS21), Quil-A, QS21-Api, and QS21-Xyl.

Aspect 9. The immunogenic composition of aspect 1 or aspect 2, wherein the stable oil-in-water emulsion comprises an aqueous phase and an oil phase, wherein the aqueous phase comprises the SLA, and wherein the oil phase comprises squalene, wherein the squalene is present in the emulsion at a concentration of from about 0.01% to about 1% v/v, and wherein the hydrophobic:lipophilic balance of the emulsion is greater than about 9.

Aspect 10. The immunogenic composition of aspect 9, wherein the aqueous phase comprises poloxamer 188 and glycerol.

Aspect 11. The immunogenic composition of aspect 9 or aspect 10, wherein the oil phase comprises egg phosphatidyl choline or dimyristoylphosphatidylcholine (DMPC) or DOPC.

Aspect 12. The immunogenic composition of any one of aspects 9-11, comprising a surfactant.

Aspect 13. The immunogenic composition of aspect 12, wherein the surfactant is pluronic F68.

Aspect 14. The immunogenic composition of any one of aspects 1-13, wherein:
a) the TP35-NS3 T-cell epitope polypeptide comprises an amino acid sequence having at least 80% amino acid sequence identity to the amino acid sequence: KSTKVPAAYAAQGYKVLVLNPSVAATLGFGAYMSK (SEQ ID NO:79);

(SEQ ID NO: 79)
KSTKVPAAYAAQGYKVLVLNPSVAATLGFGAYMSK;

b) the TP50C T-cell epitope polypeptide comprises an amino acid sequence having at least 80% amino acid sequence identity to the amino acid sequence:

(SEQ ID NO: 87)
GVYLLPRRGPRLGVRATRKTSERSQPRGRRQPIPKARRSEGRSWAQPGYP;

c) the TP27 T-cell epitope polypeptide comprises an amino acid sequence having at least 80% amino acid sequence identity to the amino acid sequence: LEQIKGGRHLIFCHSKKKCDELAAKLR (SEQ ID NO:103); and
d) the TP48 T-cell epitope polypeptide comprises an amino acid sequence having at least 80% amino acid sequence identity to the amino acid sequence:

(SEQ ID NO: 136)
ILRRHVGPGEGAVQWMNRLIAFASRGNHVSPTHYVPESDAAARVTQIL.

Aspect 15. The immunogenic composition of any one of aspects 1-13, wherein the fusion polypeptide comprises, in order from N-terminus to C-terminus:
a) the TP35-NS3 T-cell epitope polypeptide;
b) the TP27 T-cell epitope polypeptide;
c) the TP48 T-cell epitope polypeptide; and
d) the TP50C T-cell epitope polypeptide.

Aspect 16. The immunogenic composition of any one of aspects 1-15, comprising a hepatitis C virus (HCV) E1 polypeptide, an HCV E2 polypeptide, or an HCV E1/E2 heterodimeric polypeptide.

Aspect 17. The immunogenic composition of aspect 16, wherein the HCV E1 and/or E2 polypeptide is from HCV genotype 1.

Aspect 18. The immunogenic composition of aspect 16, wherein the HCV E1 and/or E2 polypeptide is from HCV genotype 2.

Aspect 19. The immunogenic composition of aspect 16, wherein the HCV E1 and/or E2 polypeptide is from HCV genotype 3.

Aspect 20. The immunogenic composition of aspect 16, wherein the HCV E1 and/or E2 polypeptide is/are from: a) HCV genotype 1, 2, and 3; b) HCV genotype 1a, 2, and 3; c) HCV genotype 1b, 2, and 3; d) HCV genotype 1a, 1b, 2, and 3; or e) HCV genotypes 1 and 3.

Aspect 21. The immunogenic composition of any one of aspects 16-20, comprising an HCV E1/E2 heterodimeric polypeptide and one or more T-cell epitope polypeptides.

Aspect 22. The immunogenic composition of aspect 21, wherein the molar ratio of the one or more T-cell epitope polypeptides to the HCV E1/E2 heterodimeric polypeptide is from 0.1:1 to 25:1.

Aspect 23. The immunogenic composition of aspect 21, wherein the composition comprises 2 or more T-cell epitope polypeptides and wherein the molar ratio of each of the 2 or more T-cell epitope polypeptides to the HCV E1/E2 heterodimeric polypeptide is independently from 0.1:1 to 25:1.

Aspect 24. The immunogenic composition of aspect 23, wherein the molar ratio of each of the 2 or more T-cell epitope polypeptides to the HCV E1/E2 heterodimeric polypeptide is independently from 2:1 to 15:1.

Aspect 25. The immunogenic composition of aspect 23, wherein the composition comprises 4 T-cell epitope polypeptides and wherein the molar ratio of each of the 4 T-cell epitope polypeptides to the HCV E1/E2 heterodimeric polypeptide is independently from 0.1:1 to 25:1.

Aspect 26. The immunogenic composition of aspect 25, wherein the molar ratio of each of the 4 T-cell epitope polypeptides to the HCV E1/E2 heterodimeric polypeptide is independently from 0.1:1 to 0.5:1, from 0.5:1 to 1:1, from 1:1 to 5:1, from 5:1 to 10:1, from 10:1 to 25:1.

Aspect 27. The immunogenic composition of aspect 25, wherein the molar ratio of each of the 4 T-cell epitope polypeptides to the HCV E1/E2 heterodimeric polypeptide is independently 0:1:1, 0.3:1, 0.5:1, or 1:1.

Aspect 28. The immunogenic composition of aspect 26, wherein the molar ratio of one or more of the 4 T-cell epitope polypeptides to the HCV E1/E2 heterodimeric polypeptide is 3:1.

Aspect 29. The immunogenic composition of aspect 26, wherein the molar ratio of one or more of the 4 T-cell epitope polypeptides to the HCV E1/E2 heterodimeric polypeptide is 9:1.

Aspect 30. The immunogenic composition of any one of aspects 25-29, wherein the 4 T-cell epitope polypeptides are a TP50C polypeptide, a TP-35NS3 polypeptide, a TP27 polypeptide, and a TP48 polypeptide.

Aspect 31. The immunogenic composition of aspect 30, wherein:
i) the TP50C polypeptide comprises the following amino acid sequence: GVYLLPRRGPRLGVRATRKTSERSQPRGRRQPIPKARRSEGRSWAQPGYP (SEQ ID NO:87) and has a length of 50 amino acids;
ii) the TP35-NS3 polypeptide comprises the following amino acid sequence: KSTKVPAAYAAQGYKVLVLNPSVAATLGFGAYMSK (SEQ ID NO:79) and has a length of 35 amino acids;
iii) the TP27 polypeptide comprises the following amino acid sequence: LEQIKGGRHLIFCHSKKKCDELAAKLR (SEQ ID NO:103) and has a length of 27 amino acids; and
iv) the TP48 polypeptide comprises the following amino acid sequence: ILRRHVGPGEGAVQWMNRLIAFASRGNHVSPTHYVPESDAAARVTQIL (SEQ ID NO:136) and has a length of 48 amino acids.

Aspect 32. The immunogenic composition of aspect 23, wherein the composition comprises 2 T-cell epitope polypeptides and wherein the molar ratio of each of the 2 T-cell epitope polypeptides to the HCV E1/E2 heterodimeric polypeptide is independently from 0.1:1 to 25:1.

Aspect 33. The immunogenic composition of aspect 32, wherein the molar ratio of each of the 2 T-cell epitope polypeptides to the HCV E1/E2 heterodimeric polypeptide is independently from 0.1:1 to 0.5:1, from 0.5:1 to 1:1, from 1:1 to 5:1, from 5:1 to 10:1, from 10:1 to 25:1.

Aspect 34. The immunogenic composition of aspect 32, wherein the molar ratio of each of the 2 T-cell epitope polypeptides to the HCV E1/E2 heterodimeric polypeptide is independently 0:1:1, 0.3:1, 0.5:1, or 1:1.

Aspect 35. The immunogenic composition of aspect 32, wherein the molar ratio of one or more of the 2 T-cell epitope polypeptides to the HCV E1/E2 heterodimeric polypeptide is 3:1.

Aspect 36. The immunogenic composition of aspect 32, wherein the molar ratio of one or more of the 2 T-cell epitope polypeptides to the HCV E1/E2 heterodimeric polypeptide is 9:1.

Aspect 37. The immunogenic composition of any one of aspects 32-36, wherein the 2 T-cell epitope polypeptides are:
a) a TP465 polypeptide and a TP48 polypeptide;
b) a TP465 polypeptide and a TP65 polypeptide; or
c) a TP465 polypeptide and a TP156 polypeptide.

Aspect 38. The immunogenic composition of any one of aspects 1-37, further comprising an adjuvant selected from MF59, alum, a CpG oligonucleotide, a cyclic dinucleotide, 3′-O-desacyl-4′-monophosphoryl lipid A (MPL), aluminum hydroxide, aluminum phosphate, AS01, AS02, AS03, AS04, 3M-052, and combinations thereof.

Aspect 39. A method of inducing an immune response in an individual to hepatitis C virus (HCV) in an individual, the method comprising administering to the individual an effective amount of the composition of any one of aspects 1-38.

Aspect 40. The method of aspect 39, comprising:
a) administering a first dose of the composition at a first time; and
b) administering a second dose of the composition at a second time.

Aspect 41. The method of aspect 40, wherein the second time is from a period of time of from about 1 week to about 6 months from the first time.

Aspect 42. The method of any one of aspects 39-41, wherein said administering is intramuscular administration.

Aspect 43. The method of any one of aspects 39-41, wherein said administering is subcutaneous administration.

Aspect 44. A container comprising the immunogenic composition of any one of aspects 1-38.

Aspect 45. The container of aspect 44, wherein the container and the composition are sterile.

Aspect 46. The container of aspect 44 or aspect 45, wherein the container is a syringe.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

Here, a strong CD8 T cell and extremely robust CD4 T cell responses to 4TPs, in addition to strong CD4 T cell and humoral responses to E1E2, is reported, when a mixture of E1E2 and 4TPs was adjuvanted with SLA-LSQ or with SLA-SE.

### Experimental Design and Methods

A mixture of E1E2 and 4TPs (including TP50-C, TP35-NS3, TP27, and TP48) that was adjuvanted with: (i) Alum-OH/MPLA (InvivoGen; San Diego, CA 92121 USA); (ii) AddaAS03 (InvivoGen); (iii) SLA-LSQ (IDRI); or (iv) SLA-SE (IDRI) was used to immunize CB6-F1 mice intramuscularly. Three injections were applied on day 0, 14, and 42 and mice were euthanized on day 56. Blood was drawn on day 0 (before immunization) and on day 56 (two weeks after the third immunization). Sera were extracted, inactivated, stored, and used for E1E2-specific enzyme-linked immunosorbent assay (ELISA) and neutralizing assay at an appropriate time. Spleens were collected on day 56, splenocytes were isolated, and used in an intracellular cytokine assay as previously reported. Landi et al. (2017) Superior immunogenicity of HCV envelope glycoproteins when adjuvanted with cyclic-di-AMP, a STING activator or archaeosomes. Vaccine 35(50): p. 6949-6956. Short peptides spanning E1E2 and 4TPs were chemically synthesized, assembled in two separate pools, and used to restimulate splenocytes in vitro.

The amino acid sequences of the 4 TPs were as follows:

TP50-C:
(SEQ ID NO: 87)
GVYLLPRRGPRLGVRATRKTSERSQPRGRRQPIPKARRSEGRSWAQPGYP;
TP35-NS3:
(SEQ IDN O: 79)
KSTKVPAAYAAQGYKVLVLNPSVAATLGFGAYMSK;
TP27:
(SEQ ID NO: 103)
LEQIKGGRHLIFCHSKKKCDELAAKLR;
TP48:
(SEQ ID NO: 136)
ILRRHVGPGEGAVQWMNRLIAFASRGNHVSPTHYVPESDAAARVTQIL.

### Results

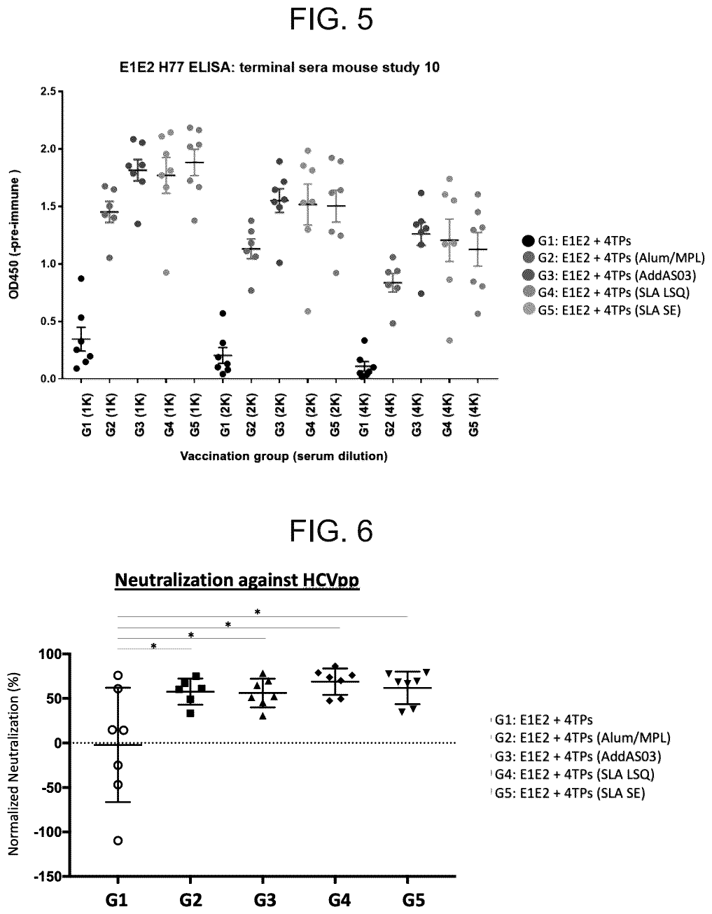

To evaluate the humoral immunogenicity of adjuvanted HCV rE1E2-4TPs vaccine, the level of E1E2-specific IgG in mice sera (FIG. 5), and neutralizing activity of post-vaccinated diluted sera against HCV pseudoparticles (FIG. 6) was compared. The results were indicative of equivalent or superior effectiveness of IDRI adjuvants (SLA-LSQ and SLA-SE) when compared to Alum-OH/MPLA and AddaAS03.

FIG. 5. E1E2-specific IgG levels in post-vaccinated mice sera collected on day 56 after subtracting the levels in pre-vaccinated mice sera collected on day 0.

FIG. 6. Diluted post-vaccination mice sera collected on day 56 was used to neutralize HCV pseudoparticles expressing E1E2 (Data are normalized to the pre-vaccinated mice sera collected on day 0).

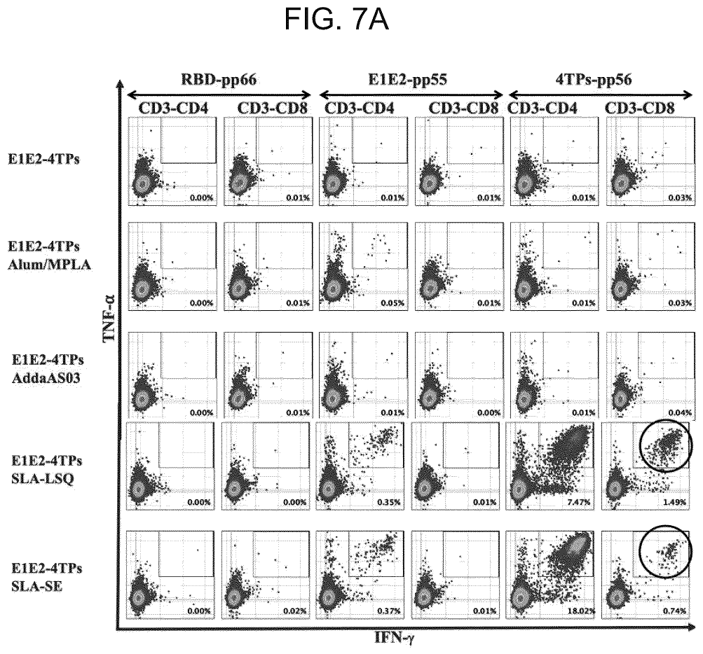

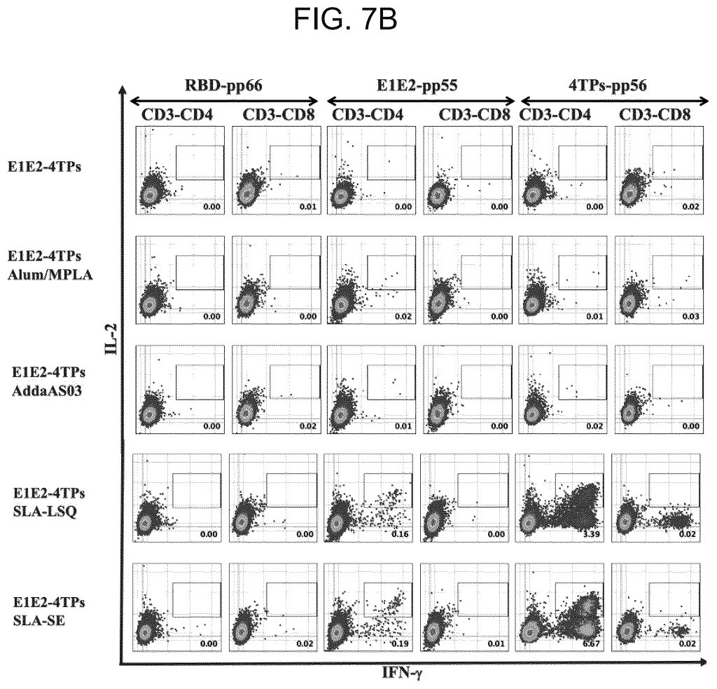

To evaluate T cell responses, the presence of polyfunctional T cells, secreting IFN-7, TNF-α, and/or IL-2, after in vitro restimulation of splenocytes was investigated. The flow cytometry data indicated that IDRI-adjuvanted vaccines induce robust and extremely robust CD4 T cells responses against E1E2 and 4TPs, respectively. Notably, IDRI adjuvants induced a strong 4TPs-specific CD8 T cells response in the preclinical animal study (FIG. 7A-7B). The CD8⁺ T cells secreted both IFN-γ and TNF-α (FIG. 7A). The CD4⁺ T cells produced all three cytokines (IFN-γ, TNF-α, and IL-2) in paired combinations of IFN-γ and TNF-α (FIG. 7A), and IFN-γ and IL-2 (FIG. 7B). The data show that the HCV vaccine induces both humoral and cellular immune responses and can induce a strong CD8 T cell response when adjuvanted with SLA-LSQ or SLA-SE.

FIG. 7A-7B. E1E2-4TPs mixture that is adjuvanted with SLA-LSQ and SLA-SE can induce the generation of CD8⁺ T cells producing IFN-γ and TNF-α (FIG. 7A) and CD4⁺ T cells producing IFN-γ, TNF-α, and IL-2 (FIGS. 7A and 7B).

### Example 2

### Methods & Materials

A mixture of adjuvanted E1E2 and TPs (the same 4 TPs as in Example 1) was used to immunize CB6-F1 mice intramuscularly. Three injections were administered on day 0, 14, and 42. Mice were euthanized on day 56 and spleens were collected. In this experiment, splenocytes from vaccinated mice with antigen (E1E2-4TPs) or adjuvanted-antigen with SLA-LSQ or SLA-SE were stimulated in vitro with full-length individual TPs, instead of peptide pools containing short peptides spanning all 4TPs. The presence of polyfunctional T cells, expressing IFN-γ, TNF-α, and/or IL-2 was investigated in an intracellular cytokine assay using flow cytometry.

### Results

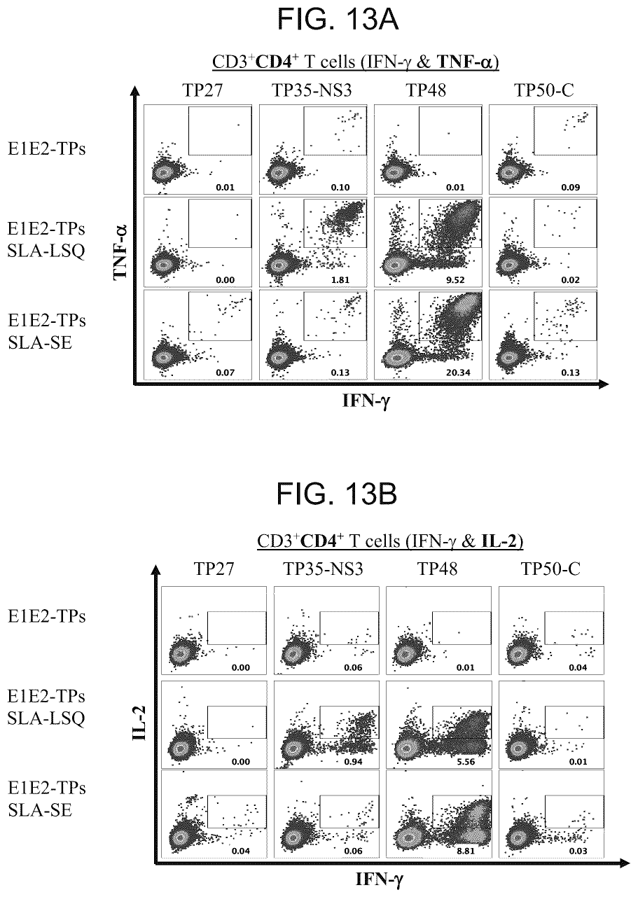

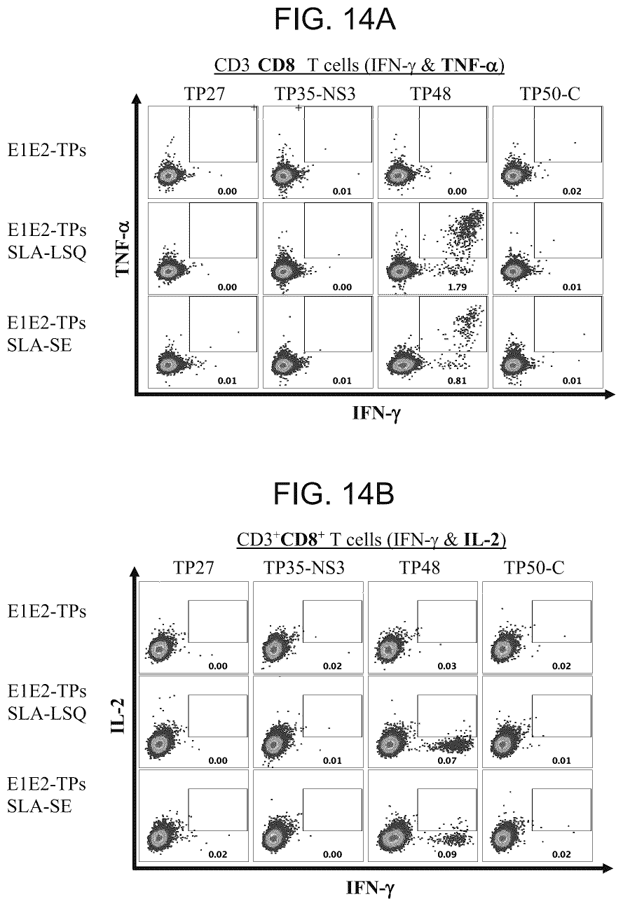

The data are depicted in FIG. 13A-13B. The flow cytometry data indicates that restimulation of solenocytes with TP48 induces an extremely strong CD4⁺ T cell response in mice receiving adjuvanted vaccine. This response is the highest among TPs, followed by TP35-NS3, TP50-C, and finally TP27. Among the TPs, TP27 induced CD4 T cells responses in the SLA-SE group. Polyfunctional CD4 T cells were able to produce all three cytokines in paired combination of IFN-γ and TNF-α (FIG. 13A), and IFN-γ and IL-2 (FIG. 13B). With respect to CD8 T cells, TP48 induced multifunctional CD8⁺ T cells (FIG. 14A), which expressed both IFN-γ and TNF-α, but not IL-2 (FIG. 14B). This HCV vaccine contains both humoral and cellular components and can induce a strong CD8 T cell response when adjuvanted with SLA-LSQ or SLA-SE.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An immunogenic composition comprising
a) one or more T-cell epitope polypeptides, wherein the T-cell epitope polypeptides are selected from:
i) a TP35-NS3 T-cell epitope polypeptide comprising the amino acid sequence:
KSTKVPX₁AYX₂X₃QGYX₄VLVLNPSVAATLGFGX₅X₆X₇SX₈ (SEQ ID NO:73), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of 35 amino acids;
ii) a TP50C T-cell epitope polypeptide comprising the amino acid sequence:
GVYX₁LPRRGPRLGVRX₂TRKX₃SERSQPRGRRQX₄IPKX₅XX₇XX₉GX₁₀X₁WX₁₂X₁₃PGYP (SEQ ID NO:80), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of 50 amino acids;
iii) a TP27 T-cell epitope polypeptide comprising the amino acid sequence:
X₁X₂X₃X₄KGGRHLIFCHSKKKCDEX₅AX₆X₇LX₈(SEQ ID NO:94), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of 27 amino acids; and
iv) a TP48 T-cell epitope polypeptide comprising the amino acid sequence:
X₁X₂X₃RHX₄GX₅X₆EGX₇X₈QWMNRLIAFASRGNHVXPTHYX₁₀X₁₁X₁₂X₃DAX₁₄X₁₅X₁₆VX₁₇X₁₈X₁₉L (SEQ ID NO:134), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein the TP48 T-cell epitope polypeptide has a length of 48 amino acids;
v) a TP240 T-cell epitope polypeptide comprising an amino acid sequence having at least 50%, at least 60%, at least 70%, at least 80%, at least 90%, or at least 95%, amino acid sequence identity to the sequence: SYQVGYLHAPTGSGKSTKVPAAYAAQGYKVLVLNPSVAATLGFGAYMSKAHGIDPNIRTGVRTVTTGA PITYSTYGKFLADGGCSGGAYDIIICDECHSTDATTILGIGTVLDQAETAGARLVVLATATPPGSVTVPHP NIEEVALGTEGEIPFYGKAIPLEVIKGGRHLIFCHSKKKCDELAAKLRGMGLNAVAYYRGLDVSVIPTSG DVVVVATDALMTGYTGDFDSVIDCNVAVTQT (SEQ ID NO:157), wherein the TP240 T-cell epitope polypeptide has a length of 240 amino acids;
vi) a TP65 T-cell epitope polypeptide comprising an amino acid sequence having at least 50%, at least 60%, at least 70%, at least 80%, at least 90%, or at least 95%, amino acid sequence identity to the sequence: TPIDTTIMAKNEVFCVDPEKGGRKPARLIVYPDLGVRVCEKMALYDVVQKLPQAVMGSSYGFQYS (SEQ ID NO:153), wherein the TP65 T-cell epitope polypeptide has a length of 65 amino acids;
vii) a TP156 T-cell epitope polypeptide comprising an amino acid sequence having at least 50%, at least 60%, at least 70%, at least 80%, at least 90%, or at least 95%, amino acid sequence identity to the sequence:
KFPGGGQIVGGVYLLPRRGPRLGVRATRKTSERSQPRGRRQPIPKARRPEGRTWAQPGYPWPLYGNEGC GWAGWLLSPRGSRPSWGPTDPRRRSRNLGKVIDTLTCGFADLMGYIPLVGAPLGGAARALAHGVRVLE DGVNYATGNLPGCSFSIFL (SEQ ID NO:160), wherein the TP156 T-cell epitope polypeptide has a length of 156 amino acids; and
viii) a TP465 T-cell epitope polypeptide comprising an amino acid sequence having at least 50%, at least 60%, at least 70%, at least 80%, at least 90%, or at least 95%, amino acid sequence identity to the amino acid sequence depicted in any one of SEQ ID NOs:58-60, wherein the TP465 T-cell epitope polypeptide has a length of 465 amino acids; and
b) a second-generation lipid adjuvant (SLA) that is a toll-like receptor 4 (TLR4) agonist formulated in: i) a stable emulsion (SE); or ii) a liposomal composition comprising a saponin (LSQ).

2. The immunogenic composition of claim 1, wherein the SLA has the following structure:

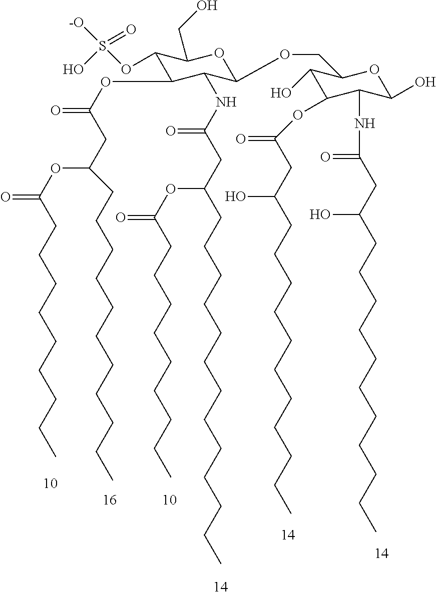

3. The immunogenic composition of claim 1, wherein the liposomal composition comprising a saponin comprises a sterol.

4. The immunogenic composition of claim 3, wherein the saponin is complexed to the sterol.

5. The immunogenic composition of claim 3, wherein the sterol is cholesterol.

6. The immunogenic composition of claim 1, wherein the liposomal composition comprises a phospholipid.

7. The immunogenic composition of claim 6, wherein the phospholipid is selected from the group consisting of DLPC, DMPC, DPPC, DSPC, DOPC, POPC, DLPG, DMPG, DPPG, DSPG, DOPG, DSTAP, DPTAP, DSPE, DPPE, DMPE, and DLPE.

8. The immunogenic composition of claim 1, wherein the saponin is QS21, QS17, QS7, synthetic QS21 (SQS21), Quil-A, QS21-Api, and QS21-Xyl.

9. The immunogenic composition of claim 1, wherein the stable emulsion comprises an aqueous phase and an oil phase, wherein the aqueous phase comprises the SLA, and wherein the oil phase comprises squalene, wherein the squalene is present in the emulsion at a concentration of from about 0.01% to about 1% v/v, and wherein the hydrophobic:lipophilic balance of the emulsion is greater than about 9.

10. The immunogenic composition of claim 9, wherein the aqueous phase comprises poloxamer 188 and glycerol.

11. The immunogenic composition of claim 9, wherein the oil phase comprises egg phosphatidyl choline or dimyristoylphosphatidylcholine (DMPC).

12. The immunogenic composition of claim 9, comprising a surfactant.

13. The immunogenic composition of claim 12, wherein the surfactant is pluronic F68.

14. The immunogenic composition of claim 1, wherein:
A) the composition comprises a TP35-NS3 T-cell epitope polypeptide, a TP50C T-cell epitope polypeptide, a TP27 T-cell epitope polypeptide, and a TP48 polypeptide, and wherein:
a) the TP35-NS3 T-cell epitope polypeptide comprises an amino acid sequence having at least 80% amino acid sequence identity to the amino acid sequence: KSTKVPAAYAAQGYKVLVLNPSVAATLGFGAYMSK (SEQ ID NO:79);
b) the TP50C T-cell epitope polypeptide comprises an amino acid sequence having at least 80% amino acid sequence identity to the amino acid sequence:
(SEQ ID NO: 87)
GVYLLPRRGPRLGVRATRKTSERSQPRGRRQPIPKARRSEGRSWAQPGYP;
c) the TP27 T-cell epitope polypeptide comprises an amino acid sequence having at least 80% amino acid sequence identity to the amino acid sequence: LEQIKGGRHLIFCHSKKKCDELAAKLR (SEQ ID NO:103); and
d) the TP48 T-cell epitope polypeptide comprises an amino acid sequence having at least 80% amino acid sequence identity to the amino acid sequence:
ILRRHVGPGEGAVQWMNRLIAFASRGNHVSPTHYVPESDAAARVTQIL (SEQ ID NO:136); or
B) the composition comprises a TP156 T-cell epitope polypeptide, a TP465 T-cell epitope polypeptide, a TP48 T-cell epitope polypeptide, and a TP65 polypeptide, and wherein:
a) the TP156 polypeptide comprises an amino acid sequence having at least 50%, at least 60%, at least 70%, at least 80%, at least 90%, or at least 95%, amino acid sequence identity to the sequence:
KFPGGGQIVGGVYLLPRRGPRLGVRATRKTSERSQPRGRRQPIPKARRPEGRTWAQPGYPWPLYGNEGC GWAGWLLSPRGSRPSWGPTDPRRRSRNLGKVIDTLTCGFADLMGYIPLVGAPLGGAARALAHGVRVLE DGVNYATGNLPGCSFSIFL (SEQ ID NO:160), wherein the TP156 T-cell epitope polypeptide has a length of 156 amino acids;
b) the TP465 polypeptide comprises an amino acid sequence having at least 50%, at least 60%, at least 70%, at least 80%, at least 90%, or at least 95%, amino acid sequence identity to the amino acid sequence depicted in any one of SEQ ID NOs:58-60, wherein the TP465 T-cell epitope polypeptide has a length of 465 amino acids;
c) the TP48 polypeptide comprises an amino acid sequence having at least 50%, at least 60%, at least 70%, at least 80%, at least 90%, or at least 95%, amino acid sequence identity to the amino acid sequence:
ILRRHVGPGEGAVQWMNRLIAFASRGNHVSPTHYVPESDAAARVTQIL (SEQ ID NO:136), wherein the TP48 polypeptide has a length of 48 amino acids; and
d) the TP65 polypeptide comprises an amino acid sequence having at least 50%, at least 60%, at least 70%, at least 80%, at least 90%, or at least 95%, amino acid sequence identity to the sequence:
TPIDTTIMAKNEVFCVDPEKGGRKPARLIVYPDLGVRVCEKMALYDVVQKLPQAVMGSSYGFQYS (SEQ ID NO:153), wherein the TP65 T-cell epitope polypeptide has a length of 65 amino acids.

15. (canceled)

16. The immunogenic composition of claim 1, comprising a hepatitis C virus (HCV) E1 polypeptide, an HCV E2 polypeptide, or an HCV E1/E2 heterodimeric polypeptide.

17. The immunogenic composition of claim 16, wherein the HCV E1 and/or E2 polypeptide is from HCV genotype 1, HCV genotype 2, or HCV genotype 3.

18.-30. (canceled)

31. The immunogenic composition of claim 14, wherein the composition comprises a TP35-NS3 T-cell epitope polypeptide, a TP50C T-cell epitope polypeptide, a TP27 T-cell epitope polypeptide, and a TP48 polypeptide, and wherein:
i) the TP50C polypeptide comprises the following amino acid sequence:
GVYLLPRRGPRLGVRATRKTSERSQPRGRRQPIPKARRSEGRSWAQPGYP (SEQ ID NO:87) and has a length of 50 amino acids;
ii) the TP35-NS3 polypeptide comprises the following amino acid sequence:
KSTKVPAAYAAQGYKVLVLNPSVAATLGFGAYMSK (SEQ ID NO:79) and has a length of 35 amino acids;
iii) the TP27 polypeptide comprises the following amino acid sequence:
LEQIKGGRHLIFCHSKKKCDELAAKLR (SEQ ID NO:103) and has a length of 27 amino acids; and
iv) the TP48 polypeptide comprises the following amino acid sequence:
ILRRHVGPGEGAVQWMNRLIAFASRGNHVSPTHYVPESDAAARVTQIL (SEQ ID NO:136) and has a length of 48 amino acids.

32.-37. (canceled)

38. The immunogenic composition of claim 1, further comprising an adjuvant selected from MF59, alum, a CpG oligonucleotide, a cyclic dinucleotide, 3′-O-desacyl-4′-monophosphoryl lipid A (MPL), aluminum hydroxide, aluminum phosphate, AS01, AS02, AS03, AS04, 3M-052, and combinations thereof.

39. A method of inducing an immune response in an individual to hepatitis C virus (HCV) in an individual, the method comprising administering to the individual an effective amount of the composition of claim 1.

40.-43. (canceled)

44. A container comprising the immunogenic composition of claim 1.

45.-46. (canceled)


SEQUENCE LISTING (172 sequences)

SEQ ID NO: 1 - protein, 746 aa, synthetic construct
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASRCWVALTPTVAXRDGSLPTTQLRRHXDLLVGSAXLCSAXYVGDLCGSVFLVGQLFTFSPRRHWTTQDCNCSIYPGHITGHRMAWDMMMNWSPTTALVVAQLLRIPQAILDMIAGAHWGVLAGIAYFSMVGNWAKVLVVLLLFAGVDAETHVTGGSAARATSGXASLFSPGAXQNIQLINTNGSWHINRTALNCNDSLDTGWVAGLFYYHKFNSSGCPERMASCRPLADFDQGWGPISYANGSGPDQRPYCWHYPPKPCGIVPAQQVCGPVYCFTPSPVVVGTTDRLGXPTYNWGENETDVLVLNNTRPPLGNWFGCTWMNSTGFTKVCGAPPCNIGGVGNNTLXCPTDCFRKHPEATYSRCGSGPWLTPRCLVDYPYRLWHYPCTVNYTIFKVRMYVGGVEHRLXAACNWTRGERCDLDDRDRSELSPLLLSTTQWQVLPCSFTTLPALSTGLIHLHQNIVDVQYLYGVGSSXVSWAIKWEYVILLFLLLADARICXCLWMMLLISQAEA

SEQ ID NO: 2 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSTGLYHVTNDCPNSSIVYEAADAILHTPGCVPCVREGNTSRCWVAMTPTVATRDGKLPTTQLRRHIDLLVGSATLCSALYVGDLCGSIFLVGQMFTFSPRRHWTTQDCNCSLYPGHITGHRMAWDMMMNWSPTAALITAQLLRIPQAILDMIAGAHWGVLAGIAYFSMVGNWAKVLVVLLLFAGVDAQTHVTGGRAAHITAGLTSLFSPGPSQKLQLVNTNGSWHINSTALNCNDSLKTGWIAGLLYSYKFNSSGCPERLASCRRLTDFAQGWGPISHANGSGPDERPYCWHYPPRPCGIVPAKSVCGPVYCFTPSPVVVGTTDKSGAPTYNWGENDTDVFVLNNTRPPLGNWFGCTWMNSTGFTKVCGAPPCVIGGAGNNTLRCPTDCFRKHPDATYSRCGSGPWITPRCLVDYPYRLWHYPCTVNYSIFKIRMYLGGVEHRLEAACNWTRGERCDLEDRDRSELSPLLLSTTQWQVLPCSFTTLPALSTGLIHLHQNIVDVQYLYGVGSSVASWAIKWDYVVLLFLLLADARICSCLWMMLLISQAEA

SEQ ID NO: 3 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 4 - protein, 746 aa, Hepatitis C virus
MSTNPKPQRKTKRNTNRRPQDVKFPGGGQIVGGVYLLPRRGPRLGVRAVRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGIYHVTNDCPNSSIVYETADTILHSPGCVPCVREGNASKCWVALAPTVATRDGKLPTTQLRRHIDLLVGSATLCSALYVGDLCGSVFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFAGVDAETYTTGGSVAQAAFGLTSLFRPGPKQDIQLINTNGSWHINRTALNCNASLDTGWVAGLFYYHKFNSSGCPERMASCRSLADFDQGWGPISYANGSGPEHRPYCWHYPPKPCGIVPAQNVCGPVYCFTPSPVVVGTTNKLGVPTYSWGSNETDVLVLNNTRPPLGNWFGCTWMNSSGFTKVCGAPPCVIGGAGNRTLHCPTDCFRKHPEATYSRCGSGPWITPRCLVHYPYRLWHYPCTVNYTMFKVRMYVGGVEHRLEVACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 5 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRSWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAFQVRNSSGLYHVTNDCPNSSIVYETADTILHSPGCVPCVREDNTSRCWVAVAPTVATRDGRLPTTQLRRHIDLLVGSATLCSALYVGDLCGSVFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFASVDAETYTSGGSVARATAGFAGIFNPGAKQDIQLINTNGSWHINRTALNCNASLDTGWVAGLFYYHKFNSSGCPERMASCKPLAHFAQGWGPISYANGSGPDHRPYCWHYPPKPCGIVPAQNVCGPVYCFTPSPVVVGTTNKLGAPTYNWGSNDTDVFILNNTRPPGGNWFGCTWMNSSGFTKVCGAPPCTIGGVGNNTLLCPTDCFRKHPEATYSRCGSGPWVTPRFLVHYPYRLWHYPCTVNYTLFKVRMYVGGVEHRLEVACNWTRGERCDLDDRDRSELSPLLLSTTQWQVLPCSFTTLPALTTGLIHLHQNIVDVQYLYGVGSSLVSWAIKWEYVILLFLLLADARICSCLWMMLLISQAEA

SEQ ID NO: 6 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNTSKCWVAVAPTVATRDGKLPTTQLRRHIDLLVGSATLCSALYVGDLCGSVFLVGQLFTFSPRRHWTTQDCNCSIYPGHITGHRMAWDMMMNWSPTAALVTAQLLRVPQAILDMIAGAHWGVLAGIAYFSMAGNWAKVLLVLLLFAGVDAETYTTGGSVARTTRGLASLLQVGPKQDIRLIHTNGSWHINRTALNCNASLDTGWLAGLLYYHKFNSSGCPERMASCRPLADFDQGWGPISYANGSGPEHRPYCWHYPPKPCGIVPAQTVCGPVYCFTPSPVVVGTTNKLGVPTYTWGSNDTDVFVLNNTRPPLGNWFGCTWMNSTGFTKVCGAPPCVIGGVGNNTLHCPTDCFRKHPEATYSRCGSGPWITPRCLVDYPYRLWHYPCTVNYTLFKVRMYVGGVEHRLQAACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 7 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREDNASRCWVPVAPTVATRDGKLPTTQLRRHIDLLVGSATLCSALYVGDLCGSVFLVSQLFTFSPRRHWTTQDCNCSIYPGHITGHRMAWDMMMNWSPTVALVMAQLLRVPQAILDMIAGAHWGVLAGIAYFSMVGNWAKVLVVLLLFAGVDAQTYVTGGSAARGASGLANLFTPGAKQDIQLINTNGSWHINRTALNCNASLDTGWVAGLFYYHKFNSSGCPERMASCKPLADFDQGWGPIRHANGSGPEHRPYCWHYPPKPCGIVSAQTVCGPVYCFTPSPVVVGTTNRLGVPTYSWGTNDTDVFVLNNTRPPLGNWFGCTWMNSSGFTKVCGAPPCVIGGVGNNTLHCPTDCFRKHPEATYSRCGSGPWITPRCLVHYPYRLWHYPCTVNYTLFKVRMYVGGVEHRLEVACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 8 - protein, 746 aa, Hepatitis C virus
MSTNPKPQRKTKRNTNRRPQDVKFPGGGQIVGGVYLLPRRGPRLGVRTTRKTSERSEPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETKDTILHSPGCVPCVREGNVSKCWVPVALTVATRDGNLPTTQLRRHIDLLVGSATLCSALYVGDLCGSVFLVGQLFTFSPRRHWTTQDCNCSMYPGHITGHRMAWDMMMNWSPTSALVVAQLLRVPQAILDMIAGAHWGVLAGLAYFSMVGNWAKVLVVLLLFASVDAGTHVTGGSAAHDVSALAGFFRRGAKQNIQLINTNGSWHVNRTALNCNASLDTGWVAGLLYYHRFNSSGCPERMASCRPLADFDQGWGPITNVDGGGSEYRPYCWHYPPKPCGIEPAQNVCGPVYCFTPSPVVVGTTDKVGVPTYNWGENDTDVFVLNNTRPPLGNWFGCTWMNSSGFVKVCGAPPCIIGGAGNKTLHCPTDCFRKHPDATYSRCGSGPWLTPRCLVDYPYRLWHYPCTVNYTLFKIRMYVGGVEHRLVAACNWTYGERCNLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 9 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVAPTVATRDGTLPTTQLRRHIDLLVGGATLCSALYVGDLCGSVFLVGQLFTFSPRRHWTTQDCNCSIYPGHITGHRMAWDMMMNWSPTTALIVAQLLRVPQAILDMIAGAHWGVLAGIAYFSMVGNWAKVLVVLLLFAGVDAETHVTGGSAAQVTSRVAGFFNPGPKQNVQLINTNGSWHINRTALNCNASLDTGWVAGLFYHYNFNSSGCPERMASCRPLADFDQGWGPISYANGSGPEHRPYCWHYPPKPCGIVPAQNVCGPVYCFTPSPVVVGTTNRLGVPTYNWGSNDTDVFVLNNTRPPLGNWFGCTWMNSSGFTKVCGAPPCVIGGVGNNTLHCPTDCFRKHPEATYSRCGSGPWITPRCLVHYPYRLWHYPCTVNYTLFKVRMYVGGVEHRLEAACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 10 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRSWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGIYHVTNDCPNSSIVYETADTILHSPGCVPCVREGNASKCWVALAPTVATRDGRLPTTQLRRHIDLLVGSATLCSALYVGDLCGSVFLVGQLFTFSPRRHWTTQDCNCSIYPGHITGHRMAWDMMMNWSPTTALVVAQLLRIPQAILDMIAGAHWGVLAGIAYFSMVGNWAKVLVVLLLFAGVDAHTRVTGGSAARATARLTTLFSPGAKQDIQLINTNGSWHINRTALNCNASLDTGWVAGLFYYHKFNSSGCPERMASCRPLADFDQGWGPISYANGSGPEHRPYCWHYPPKPCGIIPAKTVCGPVYCFTPSPVVVGTTDRSGAPTFNWGDNDTDVLVLNNTRPPLGNWFGCTWMNSSGYTKVCGAPPCIIGGVGNNTLHCPTDCFRKHPEATYSRCGSGPWITPRCLVHYPYRLWHYPCTVNYTLFKVRMYVGGVEHRLEAACNWTRGERCNLDDRDRSELSPLLLSTTQWQVLPCSFTTLPALTTGLIHLHQNIVDVQYLYGVGSSIVSWAIKWEYVILLFLLLADARVCSCLWMMLLISQVEA

SEQ ID NO: 11 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVTPTVATRDGRLPATQLRRHIDLLVGSATLCSALYVGDLCGSIFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFAGVDAGTHVTGGSAAKDTSGFTSLFRIGARQNIQLINSNGSWHINRTALNCNESLDTGWVAGLLYYHKFNSSGCPERMASCRSLADFDQGWGPISYANGSGPEHRPYCWHYPPKPCGIVPAQSVCGPVYCFTPSPVVVGTTDKSGAPTYNWGCNETDVFVLNNTRPPLGNWFGCTWMNSSGFTKVCGAPPCVIGGVGNNTLHCPTDCFRKHPEATYSRCGSGPWITPRCLVHYAYRLWHYPCTVNYTLFKVRMYVGGVEHRLDVACNWTRGERCNLDDRDRSELSPLLLSTTQWQVLPCSFTTLPALSTGLIHLHQNIVDVQYLYGVGSSIVSWAIKWEYVILLFLLLADARICSCLWMMLLISQAEA

SEQ ID NO: 12-13 - skipped (2 entries)

SEQ ID NO: 14 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADSILHSPGCVPCVREGNASKCWVAVAPTVATRDGKLPATQLRRHIDLLVGSATLCSALYVGDLCGSVFLVSQLFTFSPRRHWTTQDCNCSIYPGHITGHRMAWDMMMNWSPTTALVVSQLLRVPQAILDMIAGAHWGVLAGIAYFSMVGNWAKVLVVLLLFAGVDAETHTTGGSAAYATSGFVGLFRQGAKQNIQLINTNGSWHVNRTALNCNASLDTGWVAGLFYYHKFNSSGCPERLASCKPLANFDQGWGSISYTNGSGPEHRPYCWHYPPKPCGIVPAQNVCGPVYCFTPSPVVVGTTDRLGAPTFNWGENESDVFVLNNTRPPSGNWFGCTWMNSSGFTKVCGAPPCNIGGVGNNTLHCPTDCFRKHPEATYSRCGSGPWVTPRCLVHYPYRLWHYPCTLNYSLFKVRMYVGGIEHRLEVACNWTRGERCNLDDRDRSELSPLLLTTTQWQVLPCSFTTLPALTTGLIHLHQNVVDVQYLYGVGSSIVSWAIKWEYVILLFLLLADARICSCLWMMLLISQAEA

SEQ ID NO: 15 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVAPTVATRDGRLPTTQLRRHIDLLVGSATLCSALYVGDLCGSVFLVGQLFTFSPRHHWTTQDCNCSIYPGHITGHRMAWDMMMNWSPTTALVVAQLLRVPQAILDMIAGAHWGVLAGIAYFSMVGNWAKVLVVLLLFAGVDATTHTTGGAVAHNTRMFTSIFSLGPRQEIQLVNTNGSWHINRTALNCNASLETGWIAGLLYANRFNSSGCPERMASCKPLADFDQGWGPISYANGSGPEHRPYCWHYPPKPCGIVPAQNVCGPVYCFTPSPVVVGTTDRLGTPTYDWGSNDTDVFVLNNTRPPAGNWFGCTWMNSSGYTKVCGAPPCVIGGVSNNTLHCPTDCFRKHPEATYSRCGSGPWITPRCLVHYAYRLWHYPCTVNYTLFKVRMYVGGVEHRLEVACNWTRGERCNLDDRDRSELSPLLLSTTQWQVLPCSFTTLPALTTGLIHLHQNIVDVQYLYGVGSSIVSWAVKWEYVILLFLLLADARICSCLWMMLLISQAEA

SEQ ID NO: 16 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNASIVYEAADMIMHTPGCVPCVRENNSSRCWVALTPTLAARNASVPTTTIRRHVDLLVGAAALCSAMYVGDLCGSVFLVAQLFTFSPRRHETVQDCNCSIYPGHVTGHRMAWDMMMNWSPTAALVVSQLLRIPQAVVDMVAGAHWGVLAGLAYYSMVGNWAKVLIVMLLFAGVDGGTYVTGGTMAKNTLGITSLFSPGSSQKIQLVNTNGSWHINRTALNCNDSLNTGFLAALFYVHKFNSSGCPERMASCSPIDAFAQGWGPITYNESHSSDQRPYCWHYAPRPCGIVPAAQVCGPVYCFTPSPVVVGTTDRFGVPTYSWGENETDVLLLNNTRPPQGNWFGCTWMNSTGFTKTCGGPPCNIGGIGNKTLTCPTDCFRKHPEATYTKCGSGPWLTPRCLVHYPYRLWHYPCTVNFTIFKVRMYVGGVEHRLEAACNWTRGERCNLEDRDRSELSPLLLSTTEWQVLPCSFTTLPALSTGLIHLHQNVVDVQYLYGIGSAVVSFAIKWEYVLLLFLLLADARVCACLWMMLLIAQAEA

SEQ ID NO: 17 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGVARALAHGVRVVEDGVNYATGNLPGCSFSIFLLALLSCLTIPASAYEVRNVSGIYHVTNDCSNSSIVYEAADMIMHTPGCVPCVREGNSSRCWVALTPTLAARNASVPTTAIRRHVDLLVGAAAFCSAMYVGDLCGSVFLVSQLFTFSPRRHETIQDCNCSIYPGHVSGHRMAWDMMMNWSPTTALVVSQLLRIPQAIVDMVAGAHWGVLAGLAYYSMVGNWAKVLIVMLLFAGVDGETRVTGGQIARNAYSLTTLFSSGSAQNIQLINTNGSWHINRTALNCNDSLNTGFLAALFYTHKFNASGCPERLASCRPIDKFDQGWGPITYAEQGGQDQRPYCWHYAPKPCGIVSASKVCGPVYCFTPSPVVVGTTDRFGVPTYSWGENETDVLLLNNTRPPQGNWFGCTWMNGTGFTKTCGGPPCNIGGGGNNTLTCPTDCFRKHPAATYTKCGSGPWLTPRCLVDYPYRLWHYPCTANFTIFKVRMYVGGVEHRLDAACNWTRGERCNLEDRDRLELSPLLLSTTEWQVLPCSFTTLPALSTGLIHLHQNIVDVQYLYGIGSAVVSFAIKWDYIVILFLLLADARVCACLWMMLLIAQAEA

SEQ ID NO: 18 - protein, 747 aa, Hepatitis C virus
MSTNPKPQRKTKRNTNRRPQDVKFPGGGQIVGGVYLLPRRGPRLGVRAIRKTSERSQPRGRRQPIPKARRPEGRAWAQPGYPWPLYGNEGMGWAGWLLSPRGSRPSWGPTDPRRRSRNLGKVIDTLTCGLADLMGYIPLVGGPLGGAARALAHGVRVLEDGVNYATGNLPGCSFSIFLLALLSCLTVPASAHEVRNASGVYHVTNDCSNSSIVFEAADLIMHTPGCVPCVREGNSSRCWVALTPTLAARNATIPTTTIRHHVDLLVGAAALCSAMYVGDLCGSVFLVSQLFTFSPRRHATLQDCNCSIYPGHASGHRMAWDMMMNWSPTTALVVSQLLRIPQAVIDMVAGAHWGVLAGLAYYSMAGNWAKVLIVMLLFAGVDGHTLTTGGHAARLTSGFAGLFTPGPSQRIQLINTNGSWHINRTALNCNDSLQTGFLAALFYAHRFNSSGCPERMASCRSIDKFDQGWGPITYAEPTKDPDQRPYCWHYPPQQCGIVPASQVCGPVYCFTPSPVVVGTTDRLGNPTYSWGENDTDVLLLNNTRPPQGNWFGCTWMNSTGFTKTCGAPPCNIGGVGNNTLTCPTDCFRKHPEATYSKCGSGPWLTPRCMVDYPYRLWHYPCTVNFSIFKVRMYVGGVEHRLNAACNWTRGERCNLDDRDRSELSPLLLSTTEWQVLPCSFTTLPALSTGLIHLHQNIVDVQYLYGIGSAVVSFAIKWEYVVLLFLLLADARVCACLWMMLLIAQAEA

SEQ ID NO: 19 - protein, 746 aa, Hepatitis C virus
MSTNPKPQRKTKRNTNRRPQDVKFPGGGQIVGGVYLLPRRGPRLGVRATRKTSERSQPRGRRQPIPKARHPEGRTWAQPGYPWPLYGNEGLGWAGWLLSPRGSRPSWGPTDPRRRSRNLGKVIDTLTCGFADLMGYIPLVGAPLGGVARALAHGVRVLEDGVNYATGNLPGCSFSIFLLALLSCLTIPASAYEVRNVSGVYHVTNDCSNSSIVYEAADVIMHTPGCVPCVQDGNTSRCWVALTPTLAARNASVPVTAIRRHVDLLVGTAAFCSAMYVGDLCGSVFPVSQLFTFSPRRHQTVQDCNCSIYPGHISGHRMAWDMMMNWSPTAALVVSQLLRIPQAIVDMVAGAHWGVLAGLAYYSMVGNWAKVMIVLLLFAGVDGTTHTTGGAAARATQGFTSFFSLGPSQKIQLINTNGSWHINRTALNCNDSLQTGFLAALFYTYRFNASGCPERMASCRPIDKFDQGWGPITYAEPDSSDQRPYCWHYAPRPCGIVPASQVCGPVYCFTPSPVVVGTTDRFGVPTYTWGENETDVLLLNNTRPPLGNWFGCTWMNSTGFTKTCGGPPCNIGGAGNTTLTCPTDCFRKHPEATYTKCGSGPWLTPRCLVDYPYRLWHYPCAVNFTIFKVRMYVGGVEHRLNAACNWTRGERCDLEDRDRSELSPLLLSTTEWQVLPCSFTTLPALSTGLIHLHQNIVDVQYLYGIGSAVIPFAIKWEYVLLLFLLLADARVCACLWMMLLIAQAEA

SEQ ID NO: 20 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGMGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTTPASAYEVRNVSGIYHVTNDCSNSSIVYEAADIIMHTPGCVPCVREKNISRCWVALTPTLAARNISVPTATIRRHVDLLVGTAAFCSAMYVGDLCGSVFLVSQLFTFSPRWHETVQDCNCSLYPGHVSGHRMAWDMMMNWSPTAALVVSQLLRIPQAVVDMVAGAHWGVLAGLAYYSMIGNWAKVLIVMLLFAGADGTTHVTGGVQAHGAYGLASLFNVGPHQKIQLVNTNGSWHINRTALNCNDTLQTGFLAALFYKHRFNASGCPERMASCRPIDKFAQGWGPITYAEPDRLDQRPYCWHYPPRPCGIVPALEVCGPVYCFTPSPVVVGTTDRFGVPTYSWGENETDVLLLNNTRPPQGNWFGCTWMNSTGYTKTCGGPPCNIGGVGNNTLTCPTDCFRKHPEATYTKCGSGPWLTPRCLVHYPYRLWHYPCTVNFTIFKVRMYVGGIEHRLDAACNWTRGERCDLEDRDRSELSPLLLSTTEWQILPCSFTTLPALSTGLIHLHRNIVDVQYLYGIGSAVVSFAIKWEYVLLLFLLLADARVCACLWMMLLIAQAEA

SEQ ID NO: 21 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGLGWAGWLLSPRGSRPSWGPNDPRRRSRNLGKVIDTLTCGFADLMGYIPLVGAPLGGAARALAHGVRVLEDGVNYATGNLPGCSFSIFLLALLSCLTIPASAYEVRNVSGIYHVTNDCSNSSIVYEAADVIMHAPGCVPCVRENNSSRCWVALTPTLAARNASVPTTTLRRHVDLLVGTAAFCSAMYVGDLCGSVFLISQLFTFSPRRHETVQDCNCSIYPGHVSGHRMAWDMMMNWSPTAALVVSQLLRIPQAVMDMVAGAHWGVLAGLAYYSMVGNWAKVLIVMLLFAGVDGHTRVTGGVQGHVTSTLTSLFRPGASQKIQLVNTNGSWHINRTALNCNDSLKTGFLAALFYTHKFNASGCPERMASCRSIDKFDQGWGPITYAQPDNSDQRPYCWHYAPRQCGIVPASQVCGPVYCFTPSPVVVGTTDRFGAPTYNWGDNETDVLLLNNTRPPHGNWFGCTWMNSTGFTKTCGGPPCNIRGVGNNTLTCPTDCFRKHPDATYTKCGSGPWLTPRCLVDYPYRLWHYPCTVNFTIFKVRMYVGGVEHRLDAACNWTRGERCDLEDRDRAELSPLLLSTTEWQILPCSYTTLPALSTGLIHLHQNIVDIQYLYGIGSAVVSIAIKWEYVVLLFLLLADARVCACLWMMLLIAQAEA

SEQ ID NO: 22 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGDEGMGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTTPASAYEVRNVSGIYHVTNDCSNSSIVYEAADIIMHTPGCVPCVREKNTSRCWVALTPTLAARNISVPTTTIRRHVDLLVGTAAFCSAMYVGDLCGSVFLVSQLFTFSPRRHETVQDCNCSLYPGHVSGHRMAWDMMMNWSPTAALVVSQLLRIPQAIVDMVAGAHWGVLAGLAYYSMVGNWAKVLIVMLLFAGADGTTHVTGGVQAHGAYGLASLFNVGPHQKIQLVNTNGSWHINRTALNCNDTLQTGFLAALFYKHRFNASGCPERMASCRPIDKFAQGWGPITYAEPDRLDQRPYCWHYPPRPCGIVPALEVCGPVYCFTPSPVVVGTTDRFGVPTYSWGENETDVLLLNNTRPPQGNWFGCTWMNNTGFTKTCGGPPCNIGGVGNNTLTCPTDCFRKHPEATYTKCGSGPWLTPRCLVDYPYRLWHYPCTVNFTVFKVRMYVGGIEHRLDAACNWTRGERCDLEDRDRSELSPLLLSTTEWQILPCSFTTLPALSTGLIHLHRNIVDVQYLYGIGSAVVSFAIKWEYILLLFLLLADARVCACLWMMLLIAQAEA

SEQ ID NO: 23 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNSSIVYEAADMIMHTPGCVPCVREDNSSRCWVALTPTLAARNSSVPTTTIRRHVDLLVGAAAFCSAMYVGDLCGSVFLISQLFTFSPRRYETVQDCNCSLYPGHVSGHRMAWDMMMNWSPTTALVVSQLLRIPQAVVDMVAGAHWGVLAGLAYYSMVGNWAKVLIVMLLFAGVDGNTRVSGGEAAKNTMGFASLFVSGPSQKIQLINTNGSWHINRTALNCDDSLHTGFLAALFYAHKFNSSGCSGRMASCRPIDEFAQGWGPITHGVPDNLDQRPYCWHYAPRPCGIVPASQVCGPVYCFTPSPVVVGTTDRFGAPTYSWGENETDVLLLNNTRPPQGNWFGCTWMNSTGFTKTCGGPPCNIGGVGNNTLTCPTDCFRKHPEATYTKCGSGPWLTPRCMVDYPYRLWHYPCTVNFTIFKVRMYVGGVEHRLDAACNWTRGERCNVEDRDRSELSPLLLSTTEWQILPCSFTTLPALSTGLIHLHQNIVDVQYLYGVGSVVVSVVIRWEYVVLLFLLLADARVCACLWMMLLIAQAEA

SEQ ID NO: 24 - protein, 746 aa, Hepatitis C virus
MSTNPKPQRKTKRNTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGMYHVTNDCSNSSIVYEAADMILHAPGCVPCVRENNSSRCWVALTPTLAARNASVPTTAIRRHVDLLVGAAAFCSAMYVGDLCGSVLLVSQIFTFSPRRHETMQDCNCSIYPGHITGHRMAWDMMMNWSPTTALVVSQLLRIPQAIVDMVAGAHWGVLAGLAYYSMVGNWAKVLIVMLLFAGVDGGTYVTGGEAGRRTSGFASIFTPGASQNIQLINTNGSWHINRTALNCNDSLHTGFIAALFYHHKFNASGCPERMASCRPIGEFAQGWGPISYTEPPSSDQRPYCWHYPPRPCGIVPASQVCGPVYCFTPSPVVVGTTDRLGAPTYNWGDNDTDVLLLNNTRPPQGNWFGCTWMNGTGFTKTCGGPPCNIGGVGNNTLTCPTDCFRKHPEATYTKCGSGPWLTPRCIVDYPYRLWHYPCTVNFTITKIRMYVGGVEHRLTAACNWTRGERCDLEDRDRSELSPLLLSTTEWQIMPCSFTTLPALSTGLIHLHQNIVDIQYLYGIGSAAVSFAIRWEYVLLLFLLLADARVCACLWMMLLIAQAEA

SEQ ID NO: 25 - skipped

SEQ ID NO: 26 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRAWAQPGYPWPLYGNEGLGWAGWLLSPRGSRPSWGPTDPRRRSRNLGKVIDTLTCGFADLMGYIPLVGAPLGGTARALAHGVRVLEDGVNYATGNLPGCSFSIFLLALLSCLTIPASAYEVRNVSGVYHVTNDCSNSSIVYETADMIMHTPGCVPCVREDNFTRCWVALTPTLAARNGSVPTTAIRRHVDLLVGAAAFCSAMYVGDLCGSVFLVSQLFTFSPRWHETVQECNCSIYPGHVTGHRMAWDMMMNWSPTTALVVSQLLRIPQAVVDMVAGAHWGVLAGLAYYSMVGNWAKVLIVTLLFAGVDGNTHTIGGKQAQATGGFVAWLARGPSQEIQLINTNGSWHINRTALNCNDSLKTGFIAALFYAHRFNSSGCPERMASCRPIDKFAQGWGPITYAKPDSLDQRPYCWHYAPQPCGIVPASEVCGPVYCFTPSPVVVGTTDRSGVPTYRWGENETDVLLLNNTRPPQGNWFGCTWMNATGFTKTCGGPPCKIGGLGNNTLTCPTDCFRKHPEATYTKCGSGPWLTPRCIVDYPYRLWHYPCTVNFTIFKVRMYVGGIEHRLSAACNWTRGERCDLEDRDRSELSPLLLSTTEWQILPCSFTTLPALSTGLIHLHQNTVDVQYLYGVGSVLVSFAIKWEYILLFFLLLADARVCACLWMMLLIAQAEA

SEQ ID NO: 27 - protein, 746 aa, Hepatitis C virus
MSTNPKPQRKTKRNTNRRPQNVKFPGGGQIVGGVCLLPRRGPRVGVRATRKTSERSQPRGRRQPIPKARRPEGRSWAQPGYPWPLYGNEGCGWAGWLLSPRGSRPSRGPSDPRRRSRNLGKVIDTLTCGFADLMGYIPLVGAPLGGAARALAHGVRVLEDGVNYATGNLPGCSFSIFLLALLSCLTVPASAVEVRNSSGIYHVTNDCPNASVVYETDSLIIHLPGCVPCVREGNASRCWVSLSPTVAAKDPGVPVNEIRRHVDLIVGAAAFCSAMYVGDLCGSIFLVGQLFTLSPRRHWTTQDCNCSIYPGHVTGHRMAWDMMMNWSPTGALVVAQLLRIPQAVLDMIAGAHWGVLAGPAYYSMVGNWAKVLVVLLLFAGVDATTQVTGGTAGRNAYRLASLFSTGPSQNIQLINSNGSWHINRTALNCNDSLHTGWVAALFYSHKFNSSGRPERMASCRPLTAFDQGWGPITYGGKASNDQRPYCWHYAPRPCGIVPAKEVCGPVYCFTPSPVVVGTTDKYGVPTYTWGENETDVLLLNNSRPPIGNWFGCTWMNSTGFTKTCGAPACNVGGSETNTLSCPTDCFRRHPDATYAKCGSGPWLNPRCMVDYPYRLWHYPCTVNYTIFKIRMFVGGIEHRLTAACNWTRGERCDLDDRDRAELSPLLLSTTQWQVLPCSFTTLPALSTGLIHLHQNIVDVQYLYGLSSVVTSWAIRWEYVVLLFLLLADARICACLWMMLLISQVEA

SEQ ID NO: 28 - protein, 746 aa, Hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTYGFADLMGYIPLVGAPLGGAARALAHGVRVLEDGVNYATGNLPGCSFSIFLLALLSCLTVPASAVGVRNSSGVYHVTNDCPNASVVYETDSLIIHLPGCVPCVREGNGSRCWVSLSPTVAAKDPGVPVNEIRRHVDLIAGAAAFCSAMYVGHLCGSIFLVGQLFTLSPRRHWTTQDCNCSIYPGHVTGHRMAWDMMMNWSPTTALVVAQLLRIPQAILDMIAGAHWGVLAGIAYFSMVGNWTKVLVVLLLFAGVDATTIVSGGSAGRSTAGLVGLFSPGARQNIQLINTNGSWHINRTALNCNDTLQTGWVAGLFYTNKFNSSGCPERLASCRPLADFDQGWGPISYTNGSGPDQRPYCWHYPPKPCGIVPAESVCGPVYCFTPSPVVVGTTDRSGAPTYNWGENETDVFVLNNTRPRLGNWFGGTWMNSTGFTKVCGAPPCAIGGVGNNTLYCPTDCFRKHPEATYSRCGSGPWITPRCLIHYPYRLWHYPCTINYTIFKIRMFVGGVEHRLDAACNWTRGERCDLDDRDRAELSPLLLSTTQWQVLPCSFTTLPALSTGLIHLHQNIVDVQYLYGLSSAVTSWVIKWEYVVLLFLLLADARICACLWMMLLISQVEA

SEQ ID NO: 29 - protein, 746 aa, Hepatitis C virus
MSTNPKPQRKTKRNTNRRPQDVKFPGGGQIVGGVYLLPRRGPRVGVRATRKTSERSQPRGRRQPIPKARRPEGRSWAQPGYPWPLYGNEGCGWAGWLLSPRGSRPSWGPSDPRRRSRNLGKVIDTLTCGFADLMGYIPLVGAPLGGAARALAHGVRVLEDGVNYATGNLPGCSFSIFLLALLSCLTVPASAVGVRNSSGVYHVTNDCPNASVVYETENLIMHLPGCVPYVREGNASRCWVSLSPTVAARDSRVPVSEVRRRVDSIVGAAAFCSAMYVGDLCGSIFLVGQIFTFSPRHHWTTQDCNCSIYPGHVTGHRMAWDMMMNWSPTGALVVAQLLRIPQAIVDMIAGAHWGVLAGLAYYSMVGNWAKVVVVLLLFAGVDAETRVTGGAAGHTAFGFASFLAPGAKQKIQLINTNGSWHINRTALNCNESLDTGWLAGLLYYHKFNSSGCPERMASCQPLTAFDQGWGPITHEGNASDDQRPYCWHYALRPCGIVPAKKVCGPVYCFTPSPVVVGTTDRAGVPTYRWGANETDVLLLNNSRPPMGNWFGCTWMNSSGFTKTCGAPACNIGGSGNNTLLCPTDCFRKHPDATYSRCGSGPWLTPRCLVDYPYRLWHYPCTVNYTIFKIRMFVGGVEHRLDAACNWTRGERCDLDDRDRAELSPLLLSTTQWQVLPCSFTTLPALSTGLIHLHQNIVDVQYLYGLSSAVTSWVIKWEYVVLLFLLLADARICACLWMMLLISQVEA

SEQ ID NO: 30 - protein, 750 aa, synthetic construct
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITVPVSAVEVKNISTSYMVTNDCSNDSITWQLQAAVLHVPGCVPCENDXNTSRCWIPVSPNVAVXXPGALTQGLRTHIDMVVMSATLCSALYVGDLCGGVMLAAQXFIVSPQRHWFVQECNCSIYPGTITGHRMAWDMMMNWSPTATMILAYAMRVPEVIIDIIFGAHWGVMFGLAYFSMQGAWAKVIVILLLXAGVDARTHTVGGSXGRTTSGXAGLFSSGPKQNIQLINTNGSWHINRTALNCNDSLQTGFIASLFYTXNFNSSGCPERLSACRGIEAFRIGWGTLQYEDNVTNPEDMRPYCWHYPPKXCGIVPARSVCGPVYCFTPSPVVVGTTDRLGVPTYTWGENETDVFLLNSTRPPRGSWFGCTWMNSTGFTKTCGAPPCRIRADFNASTDLLCPTDCFRKHPDATYJKCGSGPWLTPRCLVDYPYRLWHYPCTVNXTIFKIRMYVGGVEHRLTAACNFTRGDRCNLEDRDRXQLSPLLHSTTEWAILPCSXSDLPALSTGLLHLHQNIVDVQYMYGLSPALTKYIVRWEWVVLLFLLLADARVCACLWMLILLGQAEA

SEQ ID NO: 31 - protein, 750 aa, Hepatitis C virus
MSTNPKPQRKTKRNTNRRPEDVKFPGGGQIVGGVYLLPRRGPRLGVRTTRKTSERSQPRGRRQPIPKDRRSTGKAWGKPGRPWPLYGNEGLGWAGWLLSPRGSRPSWGPTDPRHRSRNVGKVIDTLTCGFADLMGYIPVVGAPLSGAARAVAHGVRVLEDGVNYATGNLPGFPFSIFLLALLSCITVPVSAAQVKNTSSSYMVTNDCSNDSITWQLEAAVLHVPGCVPCERVGNTSRCWVPVSPNMAVRQPGALTQGLRTHIDMVVMSATFCSALYVGDLCGGVMLAAQVFIVSPQYHWFVQECNCSIYPGTITGHRMAWDMMMNWSPTATMILAYVMRVPEVIIDIVSGAHWGVMFGLAYFSMQGAWAKVIVILLLAAGVDAGTTTVGGAVARSTNVIAGVFSHGPQQNIQLINTNGSWHINRTALNCNDSLNTGFLAALFYTNRFNSSGCPGRLSACRNIEAFRIGWGTLQYEDNVTNPEDMRPYCWHYPPKPCGVVPARSVCGPVYCFTPSPVVVGTTDRRGVPTYTWGENETDVFLLNSTRPPQGSWFGCTWMNSTGFTKTCGAPPCRTRADFNASTDLLCPTDCFRKHPDATYIKCGSGPWLTPKCLVHYPYRLWHYPCTVNFTIFKIRMYVGGVEHRLTAACNFTRGDRCDLEDRDRSQLSPLLHSTTEWAILPCTYSDLPALSTGLLHLHQNIVDVQYMYGLSPAITKYVVRWEWVVLLFLLLADARVCACLWMLILLGQAEA

SEQ ID NO: 32 - protein, 749 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITTPVSAAQVKNTSDIYMVTNDCSNDSITWQLQAAALHVPGCVPCERVGNTSRCWIPVSPNVAVRQPGALTQGLRTHIDMVVMSATLCSALYVGDLCGGVMLAAQMFIISPQHHWFVQECNCSIYPGTITGHRMAWDMMMNWSPTTTMILAYVMRVPEVIIDIIGGAHWGVMFGLAYFSMQGAWAKVVVILLLAAGVDAHTRTGSSVGYATSGIVGLFTSGPKQNIQLINTNGSWHINRTALNCNDSLNTGFIVSLFYARNFNSTGCPERLSACRGIEGFRIGWGTLQYEDNVTNPEDMRPYCWHYPPKQCGIVPAGSVCGPVYCFTPSPVVVGTTDRLGVPTYTWGENETDVFLLNSTRPPVGSWFGCTWMNSTGFTKTCGAPPCRIRADFNASTDLLCPTDCFRKHPEATYIKCGSGPWLTPRCLVDYPYRLWHYPCTVNYSIFKIRMYVGGVEHRLTAACNFTRGDRCNLEDRDRSQLTPLLHSTTEWAILPCTYSDLPALSTGLLHLHQNIVDVQYMYGLSPALTKYVVRWEWVVLLFLLLADARVCACVWMLILLGQAEA

SEQ ID NO: 33 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCVTVPVSAVQVKNTSETYMVTNDCSNDSITWQLQAAVLHVPGCVPCERTGNTSRCWIPVSPNVAVQQPGALTQGLRTHIDMVVMSATLCSALYVGDLCGGVVLAAQLFIVSPRRHWFVQECNCSIYPGAITGHRMAWDMMMNWSPTTTMVLAYAMRVPEVIIDIISGAHWGVMFGLAYFSMQGAWAKVAVILLLTAGVEARTHTTGSVAGRTTSGFAGIFTSGPKQNIQLINTNGSWHINRTALNCNDSLNTGFMAALFYTKNFNSSGCPERLSXCRNIEAFRIGWGTLQYEDDVTNPEDMRPYCWHYPPKQCGIFPAGSVCGPVYCFTPSPVVVGTTNKLGVPTYTWGENETDVFILNSTRPPRGSWFGCTWMNSTGFTKTCGAPPCRIRADFNASTDLLCPTDCFRKHPEATYIKCGSGPWLTPRCLVDYPYRLWHYPCTVNYSIFKIRMYVGGVEHRLTAACNFSRGDRCNLEDRDRSQLTPLLHSTTEWAILPCSYSDLPALSTGLLHLHQNIVDVQYMYGLTPALTKYVVRWEWVVLLFLLLADARVCACLWMLILLGQAEA

SEQ ID NO: 34 - protein, 750 aa, Hepatitis C virus
MSTNPKPQRKTKRNTSRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITVPVSAVQVKNISDSYMVTNDCSNDSITWQLQAAVLHVPGCVPCEKMGNISRCWIPVSPNVAVQRPGALTQGLRAHIDMVVMSATLCSALYVGDLCGGVMLAAQMFIVSPQHHHFVQECNCSIYPGAITGHRMAWDMMMNWSPTATMILAYAMRVPEVIIDIISGAHWGVMFGLAYFSMQGAWAKVVVILLLTAGVDAHTRSIAGSVAHATSGLAGLFTSGAKQNIQLINTNGSWHINRTALNCNDSLNTGFIASLFYTYRFNSSGCPERLSACRGIQAFRIGWGTLRYEDNVTNPEDMRPYCWHYPPKQCGIVSARSVCGPVYCFTPSPVVVGTTDRLGVPTYTWGENETDVFILNSTRPPGGSWFGCTWMNSTGFTKTCGAPPCRIRADFNASMDLLCPTDCFRKHPDATYIKCGSGPWLTPRCLVDYPYRLWHYPCTINYTIFKIRMYVGGVEHRLTAACNFTRGDPCNLEDRDRSQLSPLLHSTTEWAILPCSYSDLPALSTGLLHLHQNIVDVQYMYGLSPALTKYIVRWEWVVLLFLLLADARVCACLWMLILLGQAEA

SEQ ID NO: 35 - protein, 751 aa, Hepatitis C virus
MSTNPKPQRKTQRNTNRRPQDVKFPGGGQIVGGVYLLPRRGPRLGVRTARKTSERSQPRGRRQPIPKDRRSTGKSWGRPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITIPASAVEVKNTSTGYMVTNDCANSSITWQLHAAVLHVPGCVPCERVDNNTSRCWIPVSPNIAVQRPGALTQGLRSHIDIVVMSATLCSALYVGDLCGGVMLAAQMFVVSPEHHWFVQECNCSIYPGTITGHRMAWDMMMNWSPTATMILAYAMRVPEVIIDIIGGAHWGVMFGLAYFSMQGAWAKVVVILLLAAGVDAYTHTVGGAAASTANSIAGLLSRGPRQNLQLINSNGSWHINRTALNCHDSLQTGFITALFYARHFNSSGCPERLAACRNIEAFRVGWGALQYEDNVTNPEDMRPYCWHYPPKQCGIVPARSVCGPVYCFTPSPVVVGTTDKLGVPTYTWGENETDVFLLNSTRPPQGPWFGCTWMNSTGFTKTCGAPPCRTRADFNASTDLLCPTDCFRKHPDATYNKCGSGPWLTPRCLIDYPYRLWHYPCTVNYTTFKIRMYVGGVEHRLMAACNFTRGDSCDLSQRDRGQLSPLLHSTTEWAILPCSFSDLPALSTGLLHLHQNIVDVQYMYGLSPALTKYIVRWEWVVLLFLLLADARVCACIWMLILLGQAEA

SEQ ID NO: 36 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNFATGNLPGCSFSIFLLALLSCITTPVSAAEVKNISTGYMVTNDCTNDSITWQLQAAVLHVPGCVPCEKVGNTSRCWIPVSPNVAVQQPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIIGGAHWGVMFGLAYFSMQGAWAKVVVILLLAAGVDAQTHTVGGSTAHNARTLTGMFSLGARQKIQLINTNGSWHINRTALNCNDSLHTGFLASLFYTHSFNSSGCPERMSACRSIEAFRVGWGALQYEDNVTNPEDMRPYCWHYPPRQCGVVSASSVCGPVYCFTPSPVVVGTTDRLGAPTYTWGENETDVFLLNSTRPPQGSWFGCTWMNSTGYTKTCGAPPCRIRADFNASMDLLCPTDCFRKHPDTTYIKCGSGPWLTPRCLIDYPYRLWHYPCTVNYTIFKIRMYVGGVEHRLTAACNFTRGDRCNLEDRDRSQLSPLLHSTTEWAILPCTYSDLPALSTGLLHLHQNIVDVQFMYGLSPALTKYIVRWEWVVLLFLLLADARVCACLWMLILLGQAEA

SEQ ID NO: 37 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNFATGNLPGCSFSIFLLALLSCITTPVSAAEVKNISTGYMVTNDCTNDSITWQLQAAVLHVPGCVPCEKVGNASQCWIPVSPNVAVQRPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIISGAHWGVMFGLAYFSMQGAWAKVVVILLLAAGVDARTHTVGGSAAQTTGRLTSLFDMGPRQKIQLVNTNGSWHINRTALNCNDSLHTGFIASLFYTHSFNSSGCPERMSACRSIEAFRVGWGALQYEDNVTNPEDMRPYCWHYPPRQCGVVSAKTVCGPVYCFTPSPVVVGTTDRLGAPTYTWGENETDVFLLNSTRPPLGSWFGCTWMNSSGYTKTCGAPPCRTRADFNASTDLLCPTDCFRKHPDTTYLKCGSGPWLTPRCLIDYPYRLWHYPCTVNYTIFKIRMYVGGVEHRLTAACNFTRGDRCNLEDRDRSQLSPLLHSTTEWAILPCSYSDLPALSTGLLHLHQNIVDVQFMYGLSPALTKYIVRWEWVILLFLLLADARVCACLWMLILLGQAEA

SEQ ID NO: 38 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPTDPRHRSRNLGRVIDTITCGFADLMGYIPVVGAPVGGVARALAHGVRVLEDGINYATGNLPGCSFSIFLLALLSCVTVPVSAVEVRNISSSYYATNDCSNNSITWQLTDAVLHLPGCVPCENDNGTLHCWIQVTPNVAVKHRGALTRSLRTHVDMIVMAATACSALYVGDVCGAVMILSQAFMVSPQRHNFTQECNCSIYQGHITGHRMAWDMMLSWSPTLTMILAYAARVPELVLEIIFGGHWGVVFGLAYFSMQGAWAKVIAILLLVAGVDATTYSSGQEAGRTVAGFAGLFTTGAKQNLYLINTNGSWHINRTALNCNDSLQTGFLASLFYTHKFNSSGCPERLSSCRGLDDFRIGWGTLEYETNVTNDGDMRPYCWHYPPRPCGIVPARTVCGPVYCFTPSPVVVGTTDKQGVPTYTWGENETDVFLLNSTRPPRGAWFGCTWMNGTGFTKTCGAPPCRIRKDYNSTIDLLCPTDCFRKHPDATYLKCGAGPWLTPRCLVDYPYRLWHYPCTVNFTIFKARMYVGGVEHRFSAACNFTRGDRCRLEDRDRGQQSPLLHSTTEWAVLPCSFSDLPALSTGLLHLHQNIVDVQYLYGLSPALTRYIVKWEWVILLFLLLADARICACLWMLIILGQAEA

SEQ ID NO: 39 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPHWGPTDPRHRSRNLGKVIDTITCGFADLMGYIPVIGAPVGGVARALAHGVRVLEDGVNYATGNLPGCSFSIFLLALLSCVTVPVSAVEVRNISSSYYATNDCSNNSITWQLENAVLHLPGCVPCENDNGTLRCWTQVTPNVAVKHRGALTQNLRTHVDVIVVAATVCSALYVGDVCGAVMIASQALIVSPARHNFTQECNCSIYQGRITGHHMAWDMMLNWSPTITMILAYAARIPELVLEVIFGGHWGVMFGLAYFSMQGAWAKVIVILLLVAGVDARHHTTGLQAGKTLARVTSLFSIGAKQNIQLINTNGSWHINRTALNCNDSLQTGFIASLFYVNNINSSGCPERMSSCRELDDFRIGWGTLEYETNVTNDEDMRPYCWHYPPKPCGIVPARTVCGPVYCFTPSPIVVGTTDKQGVPTYSWGENETDVFLLNSTRPPRGSWFGCTWMNGTGFTKTCGAPPCRIRRDYNSTLDLLCPTDCFRKHPDTTYLKCGSGPWLTPKCLVEYPYRLWHYPCTVNFTIFKVRMYVGAVEHRFSAACNFTRGDRCRLEDRDRGQQSPLLHSTTEWAVLPCSFSDLPALSTGLLHLHQNIVDVQYLYGLSPAITRYIVKWEWVVLLFLLLADARVCACLWMLIILGQAEA

SEQ ID NO: 40 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPTDPRHRSRNLGRVIDTITCGFADLMGYIPVVGAPVGGVARALAHGVRVLEDGINYATGNLPGCSFSIFLLALLSCCTVPVSAVEVRNISTSYYATNDCSNTSITWQLTNAVLHLPGCVPCENDNGTLRCWIQVTPNVAVKHRGALTHNLRTHVDVIVMAATVCSALYVGDICGAVMIVSQAFIISPERHNFTQECNCSMYQGHITGHRMAWDMMLNWSPTLTMILAYAARVPELVLEVIFGGHWGVVFGLAYFSMQGAWAKVIAILLLVAGVDANTYSSGVTVGHTTSTFANIFSVGPSQKINLINTNGSWHINRTALNCNDSLQTGFLASLFYVRNFNSSGCRERLSSCRRLDDFRIGWGTLEYETNVTNDEDMRPYCWHYPPKPCGIVSARTVCGPVYCFTPSPVVVGTTDRQGVPTYSWGENETDVFLLNSTRPPRGAWFGCTWMNGTGFTKTCGAPPCRIRRDYNSTLDLLCPTDCFRKHPDTTYLKCGAGPWLTPKCLVDYPYRLWHYPCTVNFTIFKVRMYVGGVEHRLSAACNFTRGDRCGLEDRDRGQQSPLLHSTTEWAVLPCSFSDLPALSTGLLHLHQNIVDVQYLYGLSPAITRYIVKWEWVVLLFLLLADARVCACLWMLIILGQAEA

SEQ ID NO: 41 - protein, 750 aa, Hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPSDPRHRSRNLGRVIDTITCGFADLMGYIPVVGAPVGGVARALAHGVRVLEDGINYATRNLPGCSFSIFLLALLSCVTVPVSSVEIRNISTSYYATNDCSNNSITWQLTNAVLHLPGCVPCENDNGTLRCWIQVTPNVAVKHRGALTHNLRAHVDVIVMAATVCSALYVGDVCGAVMIVSQALIVSPERHNFTQECNCSIYQGHITGQRMAWDMMLNWSPTLTMILAYAARVPELVLEIVFGGHWGVVFGLAYFSMQGAWAKVIAILLLVAGVDATTYSTGATVGRTVGSFAGLFKLGAQQNVQLINTNGSWHINRTALNCNDSLHTGFMAALFYANKFNSSGCPERLSSCRGLDDFRIGWGTLEYETNVTNVEDMRPYCWHYPPKPCGIVPAQSVCGPVYCFTPSPVVVGTTDRQGVPTYNWGDNETDVFLLNSTRPPRGAWFGCTWMNGTGFTKTCGAPPCRIRKDFNSTLDLLCPTDCFRKHPDATYVKCGAGPWLTPRCLIDYPYRLWHYPCTVNFTIFKVRMYVGGVEHRFSAACNFTRGDRCRLEDRDRGQQSPLLHSTTEWAVLPCSFSDLPALSTGLLHLHQNIVDVQYLYGLSPAVTKYIVKWEWVVLLFLLLADARICACLWMLIILGQAEA

SEQ ID NO: 42 - protein, 750 aa, Hepatitis C virus
MSTDPKPQRKTKRNTNRRPQDVKFPGGGQIVGGVYLLPRRGPRLGVRAARKTSERSQPRGRRQPIPKDRRSPGKSWGKPGYPWPLYGNEGCGWAGWLLSPRGSRPTWGPTDPRHRSRNLGKVIDTITCGFADLMGYIPVVGAPVGGVARALAHGVRVLEDGINYATGNLPGCSFSIFLLALLSCLTVPASAVEVRNISSGYYXTNDCSNSSITWQLTNAVLHLPGCVPCENDNGTLRCWIQVTPNVAVKYRGALTHNLRTHVDMIVMAATVCSALYVGDVCGAVMIVSQAFIMSXERHNFTQECNCSIYQGHITGHRMAWDMMLGWSPTLTMILAYAARVPEXVLEVVFGGHWGVVFGLAYFSMQGAWAKVIAILLLVAGVDAGTYSSGATIGQGTRGLVXLFSAGPSQKISLINTNGSWHINRTXLNCNDSLQTGFIASLFYAKSFNSSGCPERLSSCRGLDDFRIGWGTLEYENNVTNDEDMRPYCWHYPPKPCGIVPARTVCGPVYCFTPSPVVVGTTDKQGVPTYSWGENETDVFLLNSTRPPQGAWFGCTWMNGTGFTKTCGAPPCRIRRDHTSTLDLLCPTDCFRKHPDTTYLKCGAGPWLTPKCLVDYPYRLWHYPCTXNFTIFKVRMYVGGVEHRFSAACNFTRGDRCRLEDRDRGQQSPLLHSTTEWAVLPCSFSDLPALSTGLLHLHQNIVDVQYLYGLSPAITKYIVKWEWVILLFLLLADARVCACLWMLIILGQAEA

SEQ ID NO: 43 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIIAGAHWGILAGLAYYSMQGNWAKVAIIMVMFSGVDATTYTTGGSAARGARGLTSLFSVGAKQKLQLVNTNGSWHINSTALNCNXSINTGFIAGLFYYHKFNSTGCPQRLSSCKPITFFXQGWGPLTDANITGPSDDKPYCWHYAPRPCDVVPASSVCGPVYCFTPSPVVVGTTDAKGVPTYTWGENETDVFLLESLRPPSGRWFGCTWMNSTGFVKTCGAPPCNIYGGGGNPHNESDLFCPTDCFRKHPEATYSRCGAGPWLTPRCMVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQAEA

SEQ ID NO: 44 - protein, 752 aa, Hepatitis C virus
MSTLPKPQRKTKRNTIRRPQDVKFPGGGQIVGGVYVLPRRGPRLGVRATRKTSERSQPRERRQPIPKARRSDGRSWAQPGYPWPLYGNEGCGWAGWLLSPRGSRPSWGPNDPRRRSRNLGKVIDTLTCGFADLMGYIPIVGAPVGGVARALAHGVRALEDGINFATGNLPGCSFSIFLLALLSCLIHPAASLEWRNTSGLYILTNDCPNSSIVYEADDVILHTPGCIPCVQDGNTSTCWTSVSPTVAVRYVGATTASIRSHVDLLVGAATLCSALYVGDMCGAVFLVGQAFTFRPRRHQTVQTCNCSLYPGHLTGHRMAWDMMMNWSPAVGMVVAHVLRMPQTVFDIIAGAHWGILAGLAYYSMQGNWAKVAIIMVMFSGVDAETHTTGGTAARNAFTLTGLFTQGARQKLELINTNGSWHINRTALNCNESLNTGFIAGLFYLHKFNSTGCPERLSSCKPITFFRQGWGSLTDANITGPSDDKPYCWHYAPRPCEVVPALNVCGPVYCFTPSPVVVGTTDRQGVPTYTWGENETDVFLLRSLRPPSGQWFGCTWMNSTGFVKTCGAPPCDIYGGGGNRCNESDLFCPTDCFRKHPEATYSRCGAGPWLTPRCLVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSGVVGWALRWEFVVLVFLLLADARVCVALWLMLMISQAEA

SEQ ID NO: 45 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLVHPAASLEWRNTSGLYVLTNDCSNSSIVYEADDVILHTPGCVPCVQDDNTSTCWTPVTPTVAVRYVGATTASIRSHVDLLVGAATLCSALYVGDMCGAVFLVGQAFTFRPRRHQTVQTCNCSLYPGHVSGHRMAWDMMMNWSPAVGMVVAHILRLPQTLFDILAGAHWGILAGLAYYSMQGNWAKVAIVMIMFSGVDAETYVTGGSVAHSARGLTSLFSMGAKQKLQLVNTNGSWHINSTALNCNESINTGFIAGLFYYHKFNSTGCPQRLSSCKPIISFRQGWGPLTDANITGPSDDRPYCWHYAPRPCSVVPASSVCGPVYCFTPSPVVVGTTDIKGKPTYNWGENETDVFLLESLRPPSGRWFGCAWMNSTGFLKTCGAPPCNIYGGEGDPENETDLFCPTDCFRKHPEATYSRCGAGPWLTPRCMVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSDMVGWALKWEFVILVFLLLADARVCVALWLMLMVSQAEA

SEQ ID NO: 46 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIMAGAHWGILAGLAYYSMQGNWAKVAIIMVMFSGVDAHTYTTGGTASRHTQAFAGLFDIGPQQKLQLVNTNGSWHINSTALNCNESINTGFIAGLFYYHKFNSTGCPQRLSSCKPITFFRQGWGPLTDANITGPSDDRPYCWHYAPRPCDIVPASSVCGPVYCFTPSPVVVGTTDARGVPTYTWGENEKDVFLLKSQRPPSGRWFGCSWMNSTGFLKTCGAPPCNIYGGEGNPHNESDLFCPTDCFRKHPETTYSRCGAGPWLTPRCMVDYPYRLWHYPCTVDFRLFKVRMFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQTEA

SEQ ID NO: 47 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGQNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVSHVLRLPQTLFDIIAGAHWGILAGLAYYSMQGNWAKVAVIMVMFSGVDAETYITGGSAAHGVSTLTSLFSSGPQQKLQLVKTNGSWHINSTALNCNESINTGFIAGLFYYHKFNSTGCPQRLSSCKPITFFRQGWGSLTDANVTGASADKPYCWHYAPRPCDVVPALNVCGPVYCFTPSPVVVGTTDRKGVPTYNWGENESDVFLLESLRPPSGRWFGCAWMNSTGFLKTCGAPPCNIYGGGGNPNNESHLFCPTDCFRKHPDATYSRCGAGPWLTPRCMVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSGMVGWALKWEFVILIFLLLADARVCVALWLMLMISQAEA

SEQ ID NO: 48 - protein, 752 aa, Hepatitis C virus
MSTLPKPQRKTKRNTIRRPQDVKFPGGGQIVGGVYVLPRRGPRLGVCATRKTSERSQPRRRRQPIPKARQSGGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQNDNISTCWTPVTPTVAVRYVGATTASIRSHVDLLVGAATMCSALYVGDMCGAVFLVGQAFTFRPRRHQTVQTCNCSLYPGHLSGHRMAWDMMMNWFPALGMAVAHVLRVPQTLFDIIAGAHWGILAGLAYYSMQGNWAKVAIIMVMFSGVDAVTYTTGGSAAHATRGLTSLFSVGAQQKLQLVNTNGSWHINSTALNCNESINTGFIAGLFYYHRFNSTGCPQRLSSCKPITFFKQGWGPLTDANISGPSDDKPYCWHYAPRPCKVVPASGVCGPVYCFTPSPVVVGTTDAKGVPTYTWGANDTDVFLLESLRPPGGRWFGCTWMNSTGFVKTCGASPCDIYGGGGNSGNESDLFCPTDCFRKHPEATYSRCGAGPWLTPRCMVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGVGSGMVGWALKWEFVILIFLLLADRRVCVALWLMLMITQAEA

SEQ ID NO: 49 - protein, 752 aa, Hepatitis C virus
MSTLPKPQRKTKRNTVCRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNVSGLYILTNDCPNSSIVYEADDVILHTPGCIPCVQDGNTSTCWTALTPTVAVRYVGATTASIRSHVDLLVGAATMCSALYVGDMCGAVFLVGQAFTFRPRRHQTVQTCNCSLYPGHLTGHRMAWDMMMNWSPAVGMVVAHVLRLPQTLFDIIAGAHWGILAGLAYYSMQGNWAKVIIIMVMFSGVDATTHVTGGTAGLTAFRLTGLFTVGPQQKLQLVNTNGSWHINRTALNCNDSLNTGFIAGLFRFHKFNSTGCPEMLSSCKPITSFKQGWGPLTDANITIPSDDRPYCWHYPPRSCEVVPALSVCGPVYCFTPSPVVVGTTDAKGVPTYTWGENETDVFLLKSLRPPGGRWFGCTWMNSTGFVQTCGAPPCNIYGGGGDLKNESDLFCPTDCFRKHPEATYSRCGAGPWLTPRCMVDYPYRLWHYPCTVNFTLFKVRMFVGGFEHRFTAACNWTRGERCDIEDRDRSELHPLLHSTTELAILPCSFTPMPALSTGLIHLHQNIVDVQYLYGVGSGMVGWAVKWEFVILVFLLLADARVCVALWLMLMISQAEA

SEQ ID NO: 50 - protein, 752 aa, Hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLQWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAAGMVVAHILRLPQTLFDIIAGAHWGILAGLAYYSMQGNWAKVAIIMVMFSGVDATTYTSGGSVAQQARGLADLFSVGAKQNLQLVNTNGSWHINSTALNCDDSINTGFIAGLFYYHKFNSTGCPQRLSDCKPITFFKQGWGPLTDANITGPSDDKPYCWHYAPRRCGVVPASSVCGPVYCFTPSPVVVGTTDAKGVPTYTWGANETDVFLLESLRPPSGRWFGCAWMNSTGFLKTCGAPPCNIYGGGGNPHNESDLFCPTDCFRKHPEATYSRCGAGPWLTPRCMVDYPYRLWHYPCTVNFTLFKMRTFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQAEA

SEQ ID NO: 51 - skipped

SEQ ID NO: 52 - protein, 752 aa, Hepatitis C virus
MSTLPKPQRKTKRNTIRRPQDVKFPGGGQIVGGVYVLPRRGPRLGVCATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLVHPAASLEWRNTSGLYVLTNDCPNSSIVYEADDVILHTPGCIPCVQDGNKSTCWTSVTPTVAVKYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIIAGAHWGILAGLAYYSMQGNWAKVAIIMVMFSGVDATTYTTGGNAARGASGIVSLFTPGAKQNLQLVNTNGSWHINRTALNCNDSINTGFIAGLIYYHKFNSTGCPQRLSSCKPITFFRQGWGSLTDANITGPSDDKPYCWHYPPRPCDTIRASSVCGPVYCFTPSPVVVGTTDAKGAPTYNWGANETDMFLLQSLRPPSGRWFGCTWMNSTGFTKTCGAPPCNIYGGGGNLNNESDLFCPTDCFRKHPEATYSRCGAGPWLTPRCLVDYPYRLWHYPCTVNFTLFRMRTFVGGFEHRFTAACNWTRGERCNIEDRDRSEQHPLLHSTTELAILPCSFTPMPALSTGLIHLHQNIVDVQYLYGIGSGVVGWALKWEFVILVFLLLADARVCVALWLMLMISQAEA

SEQ ID NO: 53 - protein, 752 aa, Hepatitis C virus
MSTLPKPQRKTKRNTIRRPQDVKFPGGGVIYVGVYVLPRRGPRLGVRATRKTSERSQPRGRRKPIPKARRSEGRSWAQPGYPWPLYGNEGCGWAGWLLSPRGSRPNWAPNDPRRRSRNLGKVIDTLTCGFADLMGYIPLVGAPLGGAARALAHGVRALEDGINFATGNLPGCSFSIFLLALFSCLIHPAASLEWRNTSGLYVLTNDCSNSSIVYEADDVILHTPGCIPCVQDGNTSTCWTPVTPTVAVRYVGATTASIRSHVDLLVGAGTMCSALYVGDMCGPVFLVGQAFTFRPRRHRTVQTCNCSLYPGHLSGQRMAWDMMMNWSPAVGMVVAHILRLPQTLFDVVAGAHWGIIAGLAYYSMQGNWAKVAIIMVMFSGVDASTHVTAGQAARNAYGITSLFSVGAKQNLQLINTNGSWHINRTALNCNESINTGFIAGLFYYHKFNSTGCPQRLSSCKPITFFKQGWGPLTDANITGPSDDKPYCWHYAPRPCGIVPALNVCGPVYCFTPSPVVVGTTDAKGAPTYTWGANKTDVFLLESLRPPSGRWFGCTWMNSTGFVKTCGAPPCNIYGDGRDAQNESDLFCPTDCFRKHPEATYSRCGAGPWLTPRCLVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGIGSGMVGWALKWEFVILIFLLLADARVCVALWLILTISQAEA

SEQ ID NO: 54 - protein, 754 aa, Hepatitis C virus
MSTLPKPKRQTKRNTLRRPKNVKFPAGGQIVGEVYVLPRRGPQLGVREVRKTSERSQPRGRRQPTPKARPREGRSWAQPGYPWPLYGNEGCGWAGWLLPPRGSRPSWGQNDPRRRSRNLGKVIDTLTCGFADLMGYIPLIGAPVGGVARALAHGVRALEDGVNYATGNLPGCSFSIFLLALFSCLTCPASSLEYRNASGLYLLTNDCSNRSIVYEADDVILHLPGCVPCVETDNNNTSCWTPISPTVAVKHPGVTTASIRNHVNMLVAPPTLCSALYVEDAFGAVSLVGQAFTFRPRQHKTVQTCNCSIYPGHVSGHRMAWDMMMNWSPAIGLVISHLMRLPQTFFDLVVGAHWGVMAGLAYFSMQGNWAKVVIVLIMFSGVDATTHTTGGSAAQATAGFTSFFTRGPSQNLQLVNSNGSWHINSTALNCNDSLNTGFIAGLFYYHKFNSSGCPERMSSCKPITYFNQGWGPLTDANINGPSEDRPYCWHYPPRPCNITKPLNVCGPVYCFTPSPVVVGTTDIKGLPTYRFGVNESDVFLLTSLRPPQGRWFGCVWMNSTGFVKTCGAPPCNIYGGMKDIEANQTHLKCPTDCFRKHHDATFTRCGSGPWLTPRCLVDYPYRLWHYPCTVNFSIFKVRMFVGGHEHRFSAACNWTRGERCDLEDRDRSEQQPLLHSTTDSLILPCSFTPMRRLSTGLIHLHQNIVDVQYLYGVGSAVVGWALKWEFVVLVFLLLADARVCVALWMMLLISQAEA

SEQ ID NO: 55 - protein, 751 aa, Hepatitis C virus
MSTLPKPQRITKRNINRRPQDVKFPGGGQIVGGVYVLPRRGPKLGVRAVRKTSERSQPRSRRQPIPRARRTEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALLSCLLTPTAGLEYRNASGLYTVTNDCSNGSIVYEAGDVILHLPGCIPCVRLNNASKCWTPVSPTVAVSRPGAATASLRTHVDMMVGAATLCSALYVGDLCGALFLVGQGFSWRHRQHWTVQDCNCSIYPGHLTGHRMAWDMMMNWSPAMTLIVSQVLRLPQTMFDLVIGAHWGVMAGVAYYSMQGNWAKVFLVLCLFSGVDASTTITGGVAASGAFTITSLFSTGAKQPLHLVNTNGSWHINRTALNCNDSLNTGFIAGLLYYHKFNSSGCVERMSACSPLDRFAQGWGPLGPANISGPSSEKPYCWHYAPRPCDTVPAQSVCGPVYCFTPSPVVVGATDKRGAPTYTWGENESDVFLLESARPPTEPWFGCTWMNGSGYVKTCGAPPCHIYGGREGKSNNSLVCPTDCFRKHPDATYNRCGAGPWLTPRCLVDYPYRLWHYPCTVNYTIFKVRMFVGGLEHRFNAACNWTRGERCNLEDRDRSEMYPLLHSTTEQAILPCSFVPIPALSTGLIHLHQNIVDVQYLYGISSGLVGWAIKWEFVILIFLLLADARVCVVLWMMMLISQAEA

SEQ ID NO: 56 - protein, 750 aa, Hepatitis C virus
MSTNPKPQRLTKRNTVRRPQNVKFPGGGQIVGGVYLLPRRGPRLGVRGTRKSSERSQPRGRRQRIPKAASSQGKAWGKPGYPWPLYGNEGCGWAGWLLSPRGSRPTWGPTDPRHRSRNLGKVIDTMTCGFADLMGYIPVLGAPLGGVARALAHGVRVLEDGVNYATGNLPGCSFSIFLLALLSCLTVPASAYEVRNSSGVYHLTNDCPNASIVYETDNAILHEPGCVPCVREGNTSRCWEPVAPTLAVRYRGALTDDLRTHIDLVVASATLCSALYVGDICGAIFIASQAVLWKPGGGRIVQDCNCSIYPGHVTGHRMAWDMMQNWAPALSMVAAYAVRVPGVIITTVAGGHWGVLFGLAYFGMAGNWAKVILIMLLMSGVDAETMAVGARAAHTTGALVSLLNPGPSQRLQLINTNGSWHINRTALNCNDSLQTGFIAALFYTHRFNSSGCPERMASCKPLSDFDQGWGPLWYNSTERPSDQRPYCWHYAPSPCGIVPAKDVCGPVYCFTPSPVVVGTTDRRGVPTYTWGENESDVFLLNSTRPPQGSWFGCSWMNTTGFTKTCGGPPCKIRPQGAQSNTSLTCPTDCFRKHPRATYSACGSGPWLTPRCMVHYPYRLWHYPCTVNFTIHKVRLYIGGVEHRLDAACNWTRGERCDLEDRDRVDMSPLLHSTTELAILPCSFVPLPALSTGLIHLHQNIVDAQYLYGLSPAIISWAIRWEWVVLVFLLLADARICACLWMMMLMAQAEA

SEQ ID NO: 57 - protein, 403 aa, synthetic construct
  VARIANT 100: note = Wherein X is T, V, or Q
  VARIANT 148: note = Wherein X is T, N, Q, H, or S
  VARIANT 162: note = Wherein X is E, S, or A
  VARIANT 163: note = Wherein X is V, T, Y, F, or L
  VARIANT 208: note = Wherein X is S, Q, or T
  VARIANT 307: note = Wherein X is E, T, or V
  VARIANT 346: note = Wherein X is S, A, or P
SYQVGYLHAPTGSGKSTKVPAAYAAQGYKVLVLNPSVAATLGFGAYMSKAHGIDPNIRTGVRTVTTGAPITYSTYGKFLADGGCSGGAYDIIICDECHSXDATTILGIGTVLDQAETAGARLVVLATATPPGSVTVPHPNIEEVALGXEGEIPFYGKAIPLXXIKGGRHLIFCHSKKKCDELAAKLRGMGLNAVAYYRGLDVSVIPTXGDVVVVATDALMTGYTGDFDSVIDCNVAVTQTILRRHVGPGEGAVQWMNRLIAFASRGNHVSPTHYVPESDAAARVTQILTPIDTTIMAKNEVFCVDPXKGGRKPARLIVYPDLGVRVCEKMALYDVVQKLPQAVMGXSYGFQYSGVYLLPRRGPRLGVRATRKTSERSQPRGRRQPIPKARRSEGRSWAQPGYP

SEQ ID NO: 58 - protein, 466 aa, synthetic construct
MSFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRI

SEQ ID NO: 59 - protein, 466 aa, synthetic construct
MSYQVGYLHAPTGSGKSTKVPAAYVAQGYNVLVLNPSVAATLGFGSFMSRAYGIDPNIRTGNRTVTTGAKLTYSTYGKFLADGGCSGGAYDVIICDECHAQDATSILGIGTVLDQAETAGVRLTVLATATPPGSITVPHSNIEEVALGSEGEIPFYGKAIPIALLKGGRHLIFCHSKKKCDELASKLRGMGLNAVAYYRGLDVSVIPTAGDVVVCATDALMTGFTGDFDSVIDCNVAVEQYVDFSLDPTFSIETRTAPQDAVSRSQRRGRTGRGRLGTYRYVTPGERPSGMFDSVVLCECYDAGCSWYDLQPAETTVRLRAYLSTPGLPVCQDHLDFWESVFTGLTHIDAHFLSQTKQQGLNFSYLTAYQATVCARAQAPPPSWDEMWKCLVRLKPTLHGPTPLLYRLGPVQNEICLTHPITKYIMACMSADLEVTTSTWVLLGGVLAALAAYCLSVGCVVIVGHI

SEQ ID NO: 60 - protein, 466 aa, synthetic construct
MTYQVGYLHAPTGSGKSTKVPVAYAAQGYKVLVLNPSVAATLGFGAYMSKAHGINPNIRTGVRTVTTGEAITYSTYGKFLADGGCAGGAYDIIICDECHAVDATTILGIGTVLDQAETAGVRLTVLATATPPGSVTTPHPNIEEVALGQEGEIPFYGRAIPLSYIKGGRHLIFCHSKKKCDELAAALRGMGLNAVAYYRGLDVSIIPTQGDVVVVATDALMTGYTGDFDSVIDCNVAVTQVVDFSLDPTFTITTQTVPQDAVSRSQRRGRTGRGRLGIYRYVSTGERASGMFDSVVLCECYDAGAAWYELTPAETTVRLRAYFNTPGLPVCQDHLEFWEAVFTGLTHIDAHFLSQTKQAGENFAYLVAYQATVCARAKAPPPSWDVMWKCLTRLKPTLVGPTPLLYRLGSVTNEVTLTHPVTKYIATCMQADLEVMTSTWVLAGGVLAAVAAYCLATGCVSIIGRL

SEQ ID NO: 61 - protein, 29 aa, synthetic construct
  VARIANT 5: note = Wherein X is E or A
  VARIANT 6: note = Wherein X is V, T, Y, F or L
AIPLXXIKGGRHLIFCHSKKKCDELAAKL

SEQ ID NO: 62 - protein, 50 aa, synthetic construct
  VARIANT 9: note = Wherein X is V or I
  VARIANT 17: note = Wherein X is R or S
  VARIANT 41: note = Wherein X is T or K
LGRWTGTYXYDHLTPLXDWAAAGLRDLAVAVEPVVFSPMEXKVITWGADT

SEQ ID NO: 63 - protein, 51 aa, synthetic construct
  VARIANT 5: note = Wherein X is E or A
  VARIANT 6: note = Wherein X is V, T, Y, F or L
  VARIANT 51: note = Wherein X is S or Q
AIPLXXIKGGRHLIFCHSKKKCDELAAKLRGMGLNAVAYYRGLDVSVIPTX

SEQ ID NO: 64 - protein, 70 aa, synthetic construct
  VARIANT 44: note = Wherein X is S or Q
KGGRHLIFCHSKKKCDELAAKLRGMGLNAVAYYRGLDVSVIPTXGDVVVVATDALMTGYTGDFDSVIDCN

SEQ ID NO: 65 - protein, 100 aa, synthetic construct
  VARIANT 5: note = Wherein X is T, N, Q, H or S
  VARIANT 19: note = Wherein X is E or A
  VARIANT 20: note = Wherein X is V, T, Y, F or L
  VARIANT 65: note = Wherein X is S or Q
VALGXEGEIPFYGKAIPLXXIKGGRHLIFCHSKKKCDELAAKLRGMGLNAVAYYRGLDVSVIPTXGDVVVVATDALMTGYTGDFDSVIDCNVAVTQTVDF

SEQ ID NO: 66 - protein, 171 aa, synthetic construct
MSTNPKPQRKTKRNTNRRPQDVKFPGGGQIVGGVYLLPRRGPRLGVRATRKTSERSQPRGRRQPIPKARRSEGRSWAQPGYPWPLYGNEGCGWAGWLLSPRGSRPSWGPTDPRRRSRNLGKVIDTLTCGFADLMGYIPLVGAPLGGVARALAHGVRVLEDGVNYATGNLPG

SEQ ID NO: 67 - protein, 228 aa, synthetic construct
  VARIANT 94: note = Wherein X is T or V
  VARIANT 142: note = Wherein X is T, N, Q, H or S
  VARIANT 156: note = Wherein X is E or A
  VARIANT 157: note = Wherein X is V, T, Y, F or L
  VARIANT 202: note = Wherein X is S or Q
LHAPTGSGKSTKVPAAYAAQGYKVLVLNPSVAATLGFGAYMSKAHGIDPNIRTGVRTVTTGAPITYSTYGKFLADGGCSGGAYDIIICDECHSXDATTILGIGTVLDQAETAGARLVVLATATPPGSVTVPHPNIEEVALGXEGEIPFYGKAIPLXXIKGGRHLIFCHSKKKCDELAAKLRGMGLNAVAYYRGLDVSVIPTXGDVVVVATDALMTGYTGDFDSVIDCN

SEQ ID NO: 68 - protein, 553 aa, synthetic construct
QAXLTRVPYFVRAHXLLRXCXLVRXXAGGXYVQMAJJKLGRWTGTYXYDHLTPLXDWAAAGLRDLAVAVEPVVFSPMEXKVITWGADTAACGDILHGLPVSARLGREILLGPADXYTSKGWRLLAPITAYAQQTRGLLGXIVTSLTGRDKNZVXGEVQVLSTATQSFLGTCIXGVXWTVYHGAGXKTLAGPKGPVTQMYTNVDQDLVGWPAPPGAKSLXPCTCGSXDLYLVTRXADVIPVRRRGDSRGSLLSPRPLSXLKGSSGGPVLCPXGHAVGIFRAAVCTRGVAKAXDFIPVEXLXTTXRSPXFSDNSTPPAVPQSYQVGYLHAPTGSGKSTKVPAAYAAQGYKVLVLNPSVAATLGFGAYMSKAHGIDPNIRTGVRTVTTGAPITYSTYGKFLADGGCSGGAYDIIICDECHSXDATTILGIGTVLDQAETAGARLVVLATATPPGSVTVPHPNIEEVALGXEGEIPFYGKAIPLXXIKGGRHLIFCHSKKKCDELAAKLRGMGLNAVAYYRGLDVSVIPTXGDVVVVATDALMTGYTGDFDSVIDCN

SEQ ID NO: 69 - protein, 778 aa, synthetic construct
YLHAPTGSGKSTKVPAAYAAQGYKVLVLNPSVAATLGFGAYMSKAHGIDPNIRTGVRTVTTGAPITYSTYGKFLADGGCSGGAYDIIICDECHSXDATTILGIGTVLDQAETAGARLVVLATATPPGSVTVPHPNIEEVALGXEGEIPFYGKAIPLXXIKGGRHLIFCHSKKKCDELAAKLRGMGLNAVAYYRGLDVSVIPTXGDVVVVATDALMTGYTGDFDSVIDCNVAVTQTVDFSLDPTFTIETXTVPQDAVSRSQRRGRTGRGRLGIYRYVXPGERPSGMFDSVVLCECYDAGCAWYELTPAETTVRLRAYXNTPGLPVCQDHLEFWEXVFTGLTHIDAHFLSQTKQAGXNFXYLVAYQATVCARAQAPPPSWDXMWKCLXRLKPTLHGPTPLLYRLGAVQNEVTLTHPXTKYIMTCMSADLEVXTSTWVLXGGVLAALAAYCLXTGCVVIVGRIHLXGKPAXXPDKEVLYZXFDEMEECASHAPYIEQGQRJAEQFKXKXLGLLQXATXQAZXIXPAVQXXWXKLEAFWAKHMWNFISGIQYLAGLSTLPGNPAVASLMAFTAAXTSPLTTSXTJLFNILGGWVAXQLAPPAXATAFVVSGLAGAAVGSIGLGKVLVDILAGYGAGVSGALVAFKIMSGEKPSTEDXVNLLPAILSPGALVVGVICAAILRRHVGPGEGAVQWMNRLIAFASRGNHVSPTHYVPESDAAARVTQJLSSLTITSLLRRLHQWIXEDCPTPCSGSWLRDIWDWVCTVLTDFKTWLSSKLXPKLP

SEQ ID NO: 70 - protein, 1987 aa, synthetic construct
APITAYAQQTRGLLGXIVTSLTGRDKNZVXGEVQVLSTATQSFLGTCIXGVXWTVYHGAGXKTLAGPKGPVTQMYTNVDQDLVGWPAPPGAKSLXPCTCGSXDLYLVTRXADVIPVRRRGDSRGSLLSPRPLSXLKGSSGGPVLCPXGHAVGIFRAAVCTRGVAKAXDFIPVEXLXTTXRSPXFSDNSTPPAVPQSYQVGYLHAPTGSGKSTKVPAAYAAQGYKVLVLNPSVAATLGFGAYMSKAHGIDPNIRTGVRTVTTGAPITYSTYGKFLADGGCSGGAYDIIICDECHSXDATTILGIGTVLDQAETAGARLVVLATATPPGSVTVPHPNIEEVALGXEGEIPFYGKAIPLXXIKGGRHLIFCHSKKKCDELAAKLRGMGLNAVAYYRGLDVSVIPTXGDVVVVATDALMTGYTGDFDSVIDCNVAVTQTVDFSLDPTFTIETXTVPQDAVSRSQRRGRTGRGRLGIYRYVXPGERPSGMFDSVVLCECYDAGCAWYELTPAETTVRLRAYXNTPGLPVCQDHLEFWEXVFTGLTHIDAHFLSQTKQAGXNFXYLVAYQATVCARAQAPPPSWDXMWKCLXRLKPTLHGPTPLLYRLGAVQNEVTLTHPXTKYIMTCMSADLEVXTSTWVLXGGVLAALAAYCLXTGCVVIVGRIHLXGKPAXXPDKEVLYZXFDEMEECASHAPYIEQGQRJAEQFKXKXLGLLQXATXQAZXIXPAVQXXWXKLEAFWAKHMWNFISGIQYLAGLSTLPGNPAVASLMAFTAAXTSPLTTSXTJLFNILGGWVAXQLAPPAXATAFVVSGLAGAAVGSIGLGKVLVDILAGYGAGVSGALVAFKIMSGEKPSTEDXVNLLPAILSPGALVVGVICAAILRRHVGPGEGAVQWMNRLIAFASRGNHVSPTHYVPESDAAARVTQJLSSLTITSLLRRLHQWIXEDCPTPCSGSWLRDIWDWVCTVLTDFKTWLSSKLXPKLPGLPFISCQKGYKGVWRGDGIMTTRCPCGANITGHVKNGSMRIXGPKTCXNXWXGTFPINXYTTGPCTPKPAPNYXXALWRVAAXEYVEVTQVGDFHYVTGXTTDNLKCPCQVPAPEFFTEVDGVRLHRFAPXCKPLLRDEVTFXVGLNSYVVGSQLPCEPEPDVAVLTSMLTDPSHITAEAAARRLARGSPPSEASSSASQLSAPSLKATCTTHHDXPDADLVDANLLWRQEMGGNITRVESEXKVVILDSFDPLRAEEDDREPSVPAEXLRKSRKFPPALPIWARPDYNPPLLEXWKRPDYXPPTVHGCALPPXKAPPVPPPRRKRTVVLTESTVSXALAELATKXFGSSXXXSXXSTGADTAXSSXXQPSDEPAXSDAESYSSMPPLEGEPGDPDLSXXGSWSTVSXXEEXSVVCCSMSYSWTGALITPCXAEEEKLPINPLSNSLLRHHNXVYSTTSRSASQRQKKVTFDRLQVLDXHYDXVLKEVKAAASKVKARLLSVEEACALTPPHSAKSKFGYGAKDVRSLSXKAVNHIRSVWKDLLEDXXTPIDTTIMAKNEVFCVDPXKGGRKPARLIVYPDLGVRVCEKMALYDVVQKLPQAVMGXSYGFQYSPXQRVEFLLKAWKSKKXPMGFSYDTRCFDSTVTEXDIRTEESIYQCCXLPPEARTAIHSLTERLYVGGPMTNSKGQNCGYRRCRASGVLTTSXGNTJTCYIKALAACKAAGLXDPTMLVCGDDLVVIXESXGXZEDXAXLRAFTEAMTRYSAPPGDPPQPEYDLELITSCSSNVSVAXDXRGKRXYYLTRDPTTPLARAAWETARHTPVNSWLGNIIMYAPTIWVRMVLMTHFFSILLAQDXLXQXLDFEMYGAXYSVEPLDLPAIIERLHGLSAFSLHSYSPXELNRVAXXLRKLGXPPLRAWRHRARAVRAXLISQGGRAAICGXYLFNWAVRTKLKLTPLPAAXQLDLSGWFTVGXGGGDIYHSVSRARPRXLLLCLLLLSVGVGIFLLPAR

SEQ ID NO: 71 - DNA, 24 bp, synthetic construct
  source 1..24: mol_type = other DNA
tcgtcgttttgtcgttttgtcgtt

SEQ ID NO: 72 - DNA, 22 bp, synthetic construct
  source 1..22: mol_type = other DNA
tgactgtgaacgttcgagatga

SEQ ID NO: 73 - protein, 35 aa, synthetic construct
  VARIANT 7: note = Wherein X is A or V
  VARIANT 10: note = Wherein X is A or V
  VARIANT 11: note = Wherein X is A or S
  VARIANT 15: note = Wherein X is K or N
  VARIANT 31: note = Wherein X is A or S
  VARIANT 32: note = Wherein X is Y or F
  VARIANT 33: note = Wherein X is M or L
  VARIANT 35: note = Wherein X is K or R
KSTKVPXAYXXQGYXVLVLNPSVAATLGFGXXXSX

SEQ ID NO: 74 - protein, 35 aa, synthetic construct
KSTKVPVAYAAQGYKVLVLNPSVAATLGFGAYLSK

SEQ ID NO: 75 - protein, 35 aa, synthetic construct
KSTKVPAAYASQGYKVLVLNPSVAATLGFGAYMSK

SEQ ID NO: 76 - protein, 35 aa, synthetic construct
KSTKVPAAYVAQGYNVLVLNPSVAATLGFGSFMSR

SEQ ID NO: 77 - protein, 35 aa, synthetic construct
KSTKVPAAYAAQGYKVLVLNPSVAATLGFGAYMSR

SEQ ID NO: 78 - protein, 35 aa, synthetic construct
KSTKVPAAYASQGYKVLVLNPSVAATLGFGSYMSK

SEQ ID NO: 79 - protein, 35 aa, synthetic construct
KSTKVPAAYAAQGYKVLVLNPSVAATLGFGAYMSK

SEQ ID NO: 80 - protein, 50 aa, synthetic construct
  VARIANT 4: note = Wherein X is L or V
  VARIANT 16: note = Wherein X is A or G
  VARIANT 20: note = Wherein X is T or S
  VARIANT 32: note = Wherein X is P or R
  VARIANT 36: note = Wherein X is A or D
  VARIANT 37: note = Wherein X is R or A
  VARIANT 38: note = Wherein X is R, Q, or S
  VARIANT 39: note = Wherein X is S or P
  VARIANT 40: note = Wherein X is E, T, or Q
  VARIANT 42: note = Wherein X is R or K
  VARIANT 43: note = Wherein X is S, T, H, or A
  VARIANT 45: note = Wherein X is A or G
  VARIANT 46: note = Wherein X is Q or K
GVYXLPRRGPRLGVRXTRKXSERSQPRGRRQXIPKXXXXXGXXWXXPGYP

SEQ ID NO: 81 - protein, 50 aa, synthetic construct
GVYLLPRRGPRLGVRATRKTSERSQPRGRRQPIPKARRPEGRTWAQPGYP

SEQ ID NO: 82 - protein, 50 aa, synthetic construct
GVYLLPRRGPRLGVRATRKTSERSQPRGRRQPIPKDRRSTGKSWGKPGYP

SEQ ID NO: 83 - protein, 50 aa, synthetic construct
GVYVLPRRGPRLGVRATRKTSERSQPRGRRQPIPKARRSEGRSWAQPGYP

SEQ ID NO: 84 - protein, 50 aa, synthetic construct
GVYLLPRRGPRLGVRATRKTSERSQPRGRRQPIPKARRPTGRSWGQPGYP

SEQ ID NO: 85 - protein, 50 aa, synthetic construct
GVYLLPRRGPRLGVRATRKTSERSQPRGRRQPIPKARQPTGRHWAQPGYP

SEQ ID NO: 86 - protein, 50 aa, synthetic construct
GVYLLPRRGPRLGVRTTRKSSERSQPRGRRQRIPKAASSQGKAWGKPGYP

SEQ ID NO: 87 - protein, 50 aa, synthetic construct
GVYLLPRRGPRLGVRATRKTSERSQPRGRRQPIPKARRSEGRSWAQPGYP

SEQ ID NO: 88 - protein, 23 aa, Hepatitis C virus
DVVVVATDALMTGFTGDFDSVID

SEQ ID NO: 89 - protein, 23 aa, synthetic construct
  VARIANT 5: note = Wherein X is V or C
  VARIANT 6: note = Wherein X is A or S
  VARIANT 14: note = Wherein X is F or Y
DVVVXXTDALMTGXTGDFDSVID

SEQ ID NO: 90 - protein, 23 aa, synthetic construct
DVVVVATDALMTGYTGDFDSVID

SEQ ID NO: 91 - protein, 23 aa, synthetic construct
DVVVCATDALMTGFTGDFDSVID

SEQ ID NO: 92 - protein, 23 aa, synthetic construct
DVVVCSTDALMTGFTGDFDSVID

SEQ ID NO: 93 - protein, 23 aa, synthetic construct
DVVVCATDALMTGYTGDFDSVID

SEQ ID NO: 94 - protein, 27 aa, synthetic construct
  VARIANT 1: note = Wherein X is L or I
  VARIANT 2: note = Wherein X is E, A, S, V, or Q
  VARIANT 3: note = Wherein X is Q, T, Y, F, or L
  VARIANT 4: note = Wherein X is I or L
  VARIANT 22: note = Wherein X is L or I
  VARIANT 24: note = Wherein X is A, K, or S
  VARIANT 25: note = Wherein X is K, Q, or A
  VARIANT 27: note = Wherein X is T, R, or S
XXXXKGGRHLIFCHSKKKCDEXAXXLX

SEQ ID NO: 95 - protein, 27 aa, synthetic construct
LEVIKGGRHLIFCHSKKKCDELAAKLV

SEQ ID NO: 96 - protein, 27 aa, synthetic construct
IETIKGGRHLIFCHSKKKCDELAAKLS

SEQ ID NO: 97 - protein, 27 aa, synthetic construct
LSYIKGGRHLIFCHSKKKCDELAAALR

SEQ ID NO: 98 - protein, 26 aa, synthetic construct
LAFIKGGRHLIFCHSKKKCDELAALR

SEQ ID NO: 99 - protein, 27 aa, synthetic construct
IAQLKGGRHLIFCHSKKKCDEIASKLR

SEQ ID NO: 100 - protein, 26 aa, synthetic construct
LELIKGGRHLIFCHSKKKCDELAQLT

SEQ ID NO: 101 - protein, 27 aa, synthetic construct
LEYIKGGRHLIFCHSKKKCDELAKQLT

SEQ ID NO: 102 - protein, 27 aa, synthetic construct
LQHIKGGRHLIFCHSKKKCDELAGKLT

SEQ ID NO: 103 - protein, 27 aa, synthetic construct
LEQIKGGRHLIFCHSKKKCDELAAKLR

SEQ ID NO: 104 - protein, 35 aa, synthetic construct
ILRRHVGPGEGAVQWMNRLIAFASRGNHVSPTHYV

SEQ ID NO: 105 - protein, 35 aa, synthetic construct
  VARIANT 1: note = Wherein X is I or V
  VARIANT 2: note = Wherein X is L or I
  VARIANT 3: note = Wherein X is R or K
  VARIANT 6: note = Wherein X is V, I, or T
  VARIANT 8: note = Wherein X is P, Q, or T
  VARIANT 9: note = Wherein X is G, A, or S
  VARIANT 12: note = Wherein X is A or V
  VARIANT 13: note = Wherein X is V or T
  VARIANT 30: note = Wherein X is S or A
  VARIANT 35: note = Wherein X is V or I
XXXRHXGXXEGXXQWMNRLIAFASRGNHVXPTHYX

SEQ ID NO: 106 - protein, 35 aa, synthetic construct
ILRRHVGPGEGAVQWMNRLIAFASRGNHVAPTHYV

SEQ ID NO: 107 - protein, 35 aa, synthetic construct
ILRRHVGQGEGAVQWMNRLIAFASRGNHVAPTHYV

SEQ ID NO: 108 - protein, 35 aa, synthetic construct
VLRRHIGPGEGAVQWMNRLIAFASRGNHVSPTHYV

SEQ ID NO: 109 - protein, 35 aa, synthetic construct
ILRRHVGPAEGATQWMNRLIAFASRGNHVSPTHYV

SEQ ID NO: 110 - protein, 35 aa, synthetic construct
IIKRHTGTSEGVTQWMNRLIAFASRGNHVSPTHYI

SEQ ID NO: 111 - protein, 42 aa, synthetic construct
GTEGEIPFYGKAIPLEQIKGGRHLIFCHSKKKCDELAAKLTG

SEQ ID NO: 112 - protein, 52 aa, synthetic construct
  VARIANT 1: note = Wherein X is G, P, or S
  VARIANT 2: note = Wherein X is T, N, Q, H, or S
  VARIANT 3: note = Wherein X is E, T, or D
  VARIANT 11: note = Wherein X is K or R
  VARIANT 15: note = Wherein X is L or I
  VARIANT 16: note = Wherein X is E, A, S, or Q
  VARIANT 17: note = Wherein X is Q, T, Y, F, or L
  VARIANT 18: note = Wherein X is I or L
  VARIANT 36: note = Wherein X is L or I
  VARIANT 38: note = Wherein X is A, K, or S
  VARIANT 39: note = Wherein X is K, Q, or A
  VARIANT 41: note = Wherein X is T, R, or S
  VARIANT 42: note = Wherein X is G or S
  VARIANT 43..52: note = Wherein up to 10 copies of K may be deleted
XXXGEIPFYGXAIPXXXXKGGRHLIFCHSKKKCDEXAXXLXXKKKKKKKKKK

SEQ ID NO: 113 - skipped

SEQ ID NO: 114 - protein, 45 aa, synthetic construct
GTEGEIPFYGKAIPLEQIKGGRHLIFCHSKKKCDELAAKLTGKKK

SEQ ID NO: 115 - protein, 42 aa, synthetic construct
STTGEIPFYGKAIPLEVIKGGRHLIFCHSKKKCDELAAKLVA

SEQ ID NO: 116 - protein, 42 aa, synthetic construct
SNTGEIPFYGKAIPIETIKGGRHLIFCHSKKKCDELAAKLSG

SEQ ID NO: 117 - protein, 42 aa, synthetic construct
GQEGEIPFYGRAIPLSYIKGGRHLIFCHSKKKCDELAAALRG

SEQ ID NO: 118 - protein, 41 aa, synthetic construct
GHEGEIPFYGKAIPLAFIKGGRHLIFCHSKKKCDELAALRG

SEQ ID NO: 119 - protein, 42 aa, synthetic construct
GSEGEIPFYGKAIPIAQLKGGRHLIFCHSKKKCDEIASKLRG

SEQ ID NO: 120 - protein, 41 aa, synthetic construct
PTTGEIPFYGKAIPLELIKGGRHLIFCHSKKKCDELAQLTS

SEQ ID NO: 121 - protein, 42 aa, synthetic construct
PSEGEIPFYGRAIPLXLIKGGRHLIFCHSKKKCDELAKQLTS

SEQ ID NO: 122 - protein, 42 aa, synthetic construct
PTTGEIPFYGKAIPLEYIKGGRHLIFCHSKKKCDELAKQLTS

SEQ ID NO: 123 - protein, 42 aa, synthetic construct
GNDGEIPFYGKAIPLQHIKGGRHLIFCHSKKKCDELAGKLTS

SEQ ID NO: 124 - protein, 37 aa, synthetic construct
IPFYGKAIPLEQIKGGRHLIFCHSKKKCDELAAKLTG

SEQ ID NO: 125 - protein, 37 aa, synthetic construct
GTEGEIPFYGKAIPLEQIKGGRHLIFCHSKKKCDELA

SEQ ID NO: 126 - skipped

SEQ ID NO: 127 - protein, 45 aa, synthetic construct
LNAVAYYRGLDVSVIPTSGDVVVVATDALMTGFTGDFDSVIDCNV

SEQ ID NO: 128 - protein, 45 aa, synthetic construct
LNAVAYYRGLDVSVIPTSGDVVVVATDALMTGFTGDFDSVIDKKK

SEQ ID NO: 129 - protein, 55 aa, synthetic construct
  VARIANT 1: note = Wherein X is L or V
  VARIANT 2: note = Wherein X is N or T
  VARIANT 6: note = Wherein X is Y or F
  VARIANT 10: note = Wherein X is L or V
  VARIANT 13: note = Wherein X is S or A
  VARIANT 14: note = Wherein X is V or I
  VARIANT 17: note = Wherein X is T or A
  VARIANT 18: note = Wherein X is S, Q, or T
  VARIANT 24: note = Wherein X is V or C
  VARIANT 25: note = Wherein X is A or S
  VARIANT 33: note = Wherein X is F or Y
  VARIANT 43: note = Wherein X is C or K
  VARIANT 44: note = Wherein X is N or K
  VARIANT 45: note = Wherein X is V or K
  VARIANT 46..55: note = Wherein up to 10 copies of K may be deleted
XXAVAXYRGXDVXXIPXXGDVVVXXTDALMTGXTGDFDSVIDXXXKKKKKKKKKK

SEQ ID NO: 130 - skipped

SEQ ID NO: 131 - protein, 48 aa, synthetic construct
LNAVAYYRGLDVSVIPTSGDVVVVATDALMTGFTGDFDSVIDCNVKKK

SEQ ID NO: 132 - skipped

SEQ ID NO: 133 - protein, 42 aa, synthetic construct
LNAVAYYRGLDVSVIPTSGDVVVVATDALMTGFTGDFDSVID

SEQ ID NO: 134 - protein, 48 aa, synthetic construct
  VARIANT 1: note = Wherein X is I or V
  VARIANT 2: note = Wherein X is L or I
  VARIANT 3: note = Wherein X is R or K
  VARIANT 6: note = Wherein X is V, I, or T
  VARIANT 8: note = Wherein X is P, Q, or T
  VARIANT 9: note = Wherein X is G, A, or S
  VARIANT 12: note = Wherein X is A or V
  VARIANT 13: note = Wherein X is V or T
  VARIANT 30: note = Wherein X is S or A
  VARIANT 35: note = Wherein X is V or I
  VARIANT 36: note = Wherein X is P, T, A, or Q
  VARIANT 37: note = Wherein X is E or D
  VARIANT 38: note = Wherein X is S, T, or D
  VARIANT 41: note = Wherein X is S or A
  VARIANT 42: note = Wherein X is A, Q, R, or K
  VARIANT 43: note = Wherein X is any amino acid
  VARIANT 45: note = Wherein X is T or M
  VARIANT 46: note = Wherein X is Q, A, T, or G
  VARIANT 47: note = Wherein X is I, L, or V
XXXRHXGXXEGXXQWMNRLIAFASRGNHVXPTHYXXXXDAXXXVXXXL

SEQ ID NO: 135 - protein, 48 aa, synthetic construct
ILRRHVGPGEGAVQWMNRLIAFASRGNHVSPTHYVPESDAAARVTAIL

SEQ ID NO: 136 - protein, 48 aa, synthetic construct
ILRRHVGPGEGAVQWMNRLIAFASRGNHVSPTHYVPESDAAARVTQIL

SEQ ID NO: 137 - protein, 48 aa, synthetic construct
ILRRHVGPGEGAVQWMNRLIAFASRGNHVAPTHYVTESDASQRVTQLL

SEQ ID NO: 138 - protein, 48 aa, synthetic construct
ILRRHVGQGEGAVQWMNRLIAFASRGNHVAPTHYVAESDASQRVTQVL

SEQ ID NO: 139 - protein, 48 aa, synthetic construct
ILRRHVGPGEGAVQWMNRLIAFASRGNHVSPTHYVPESDAAARVTALL

SEQ ID NO: 140 - protein, 48 aa, synthetic construct
VLRRHIGPGEGAVQWMNRLIAFASRGNHVSPTHYVPETDASAKVTQLL

SEQ ID NO: 141 - protein, 48 aa, synthetic construct
IIKRHTGTSEGVTQWMNRLIAFASRGNHVSPTHYIQDDDASKRVMGIL

SEQ ID NO: 142 - protein, 33 aa, synthetic construct
HSKKKCDELAAKLTGLGLNAVAYYRGLDVSVIP

SEQ ID NO: 143 - protein, 33 aa, synthetic construct
  VARIANT 11: note = Wherein X is A or S
  VARIANT 12: note = Wherein X is K or A
  VARIANT 14: note = Wherein X is V, S, R, or T
  VARIANT 15: note = Wherein X is A or G
  VARIANT 16: note = Wherein X is L or M
  VARIANT 18: note = Wherein X is I, L, or V
  VARIANT 31: note = Wherein X is V or I
HSKKKCDELAXXLXXXGXNAVAYYRGLDVSXIP

SEQ ID NO: 144 - protein, 33 aa, synthetic construct
  VARIANT 14: note = Wherein X is V, S, R, or T
  VARIANT 15: note = Wherein X is A or G
  VARIANT 16: note = Wherein X is L or M
  VARIANT 18: note = Wherein X is I, L, or V
HSKKKCDELAAKLXXXGXNAVAYYRGLDVSVIP

SEQ ID NO: 145 - protein, 33 aa, synthetic construct
  VARIANT 18: note = Wherein X is I, L, or V
  VARIANT 31: note = Wherein X is V or I
HSKKKCDELAAALRGMGXNAVAYYRGLDVSXIP

SEQ ID NO: 146 - protein, 33 aa, synthetic construct
HSKKKCDELAAKLRGMGLNAVAYYRGLDVSVIP

SEQ ID NO: 147 - protein, 42 aa, synthetic construct
KGGRHLIFCHSKKKCDELAAKLTGLGLNAVAYYRGLDVSVIP

SEQ ID NO: 148 - protein, 42 aa, synthetic construct
  VARIANT 20: note = Wherein X is A or S
  VARIANT 21: note = Wherein X is K or A
  VARIANT 23: note = Wherein X is V, S, R, or T
  VARIANT 24: note = Wherein X is A or G
  VARIANT 25: note = Wherein X is L or M
  VARIANT 27: note = Wherein X is I, L, or V
  VARIANT 40: note = Wherein X is V or I
KGGRHLIFCHSKKKCDELAXXLXXXGXNAVAYYRGLDVSXIP

SEQ ID NO: 149 - protein, 42 aa, synthetic construct
  VARIANT 23: note = Wherein X is V, S, R, or T
  VARIANT 24: note = Wherein X is A or G
  VARIANT 25: note = Wherein X is L or M
  VARIANT 27: note = Wherein X is I, L, or V
KGGRHLIFCHSKKKCDELAAKLXXXGXNAVAYYRGLDVSVIP

SEQ ID NO: 150 - protein, 42 aa, synthetic construct
  VARIANT 27: note = Wherein X is I, L, or V
  VARIANT 40: note = Wherein X is V or I
KGGRHLIFCHSKKKCDELAAALRGMGXNAVAYYRGLDVSXIP

SEQ ID NO: 151 - protein, 42 aa, synthetic construct
KGGRHLIFCHSKKKCDELAAKLRGMGLNAVAYYRGLDVSVIP

SEQ ID NO: 152 - protein, 65 aa, synthetic construct
  VARIANT 19: note = Wherein X is E, T, or V
  VARIANT 58: note = Wherein X is S, A, or P
TPIDTTIMAKNEVFCVDPXKGGRKPARLIVYPDLGVRVCEKMALYDVVQKLPQAVMGXSYGFQYS

SEQ ID NO: 153 - protein, 65 aa, synthetic construct
TPIDTTIMAKNEVFCVDPEKGGRKPARLIVYPDLGVRVCEKMALYDVVQKLPQAVMGSSYGFQYS

SEQ ID NO: 154 - protein, 65 aa, synthetic construct
TPIDTTIMAKNEVFCVDPTKGGRKPARLIVYPDLGVRVCEKMALYDVVQKLPQAVMGASYGFQYS

SEQ ID NO: 155 - protein, 65 aa, synthetic construct
TPIDTTIMAKNEVFCVDPVKGGRKPARLIVYPDLGVRVCEKMALYDVVQKLPQAVMGPSYGFQYS

SEQ ID NO: 156 - protein, 240 aa, synthetic construct
  VARIANT 100: note = Wherein X is T, V, or Q
  VARIANT 148: note = Wherein X is T, N, Q, H, or S
  VARIANT 162: note = Wherein X is E, S, or A
  VARIANT 163: note = Wherein X is V, T, Y, F, or L
  VARIANT 208: note = Wherein X is S, Q, or T
SYQVGYLHAPTGSGKSTKVPAAYAAQGYKVLVLNPSVAATLGFGAYMSKAHGIDPNIRTGVRTVTTGAPITYSTYGKFLADGGCSGGAYDIIICDECHSXDATTILGIGTVLDQAETAGARLVVLATATPPGSVTVPHPNIEEVALGXEGEIPFYGKAIPLXXIKGGRHLIFCHSKKKCDELAAKLRGMGLNAVAYYRGLDVSVIPTXGDVVVVATDALMTGYTGDFDSVIDCNVAVTQT

SEQ ID NO: 157 - protein, 240 aa, synthetic construct
SYQVGYLHAPTGSGKSTKVPAAYAAQGYKVLVLNPSVAATLGFGAYMSKAHGIDPNIRTGVRTVTTGAPITYSTYGKFLADGGCSGGAYDIIICDECHSTDATTILGIGTVLDQAETAGARLVVLATATPPGSVTVPHPNIEEVALGTEGEIPFYGKAIPLEVIKGGRHLIFCHSKKKCDELAAKLRGMGLNAVAYYRGLDVSVIPTSGDVVVVATDALMTGYTGDFDSVIDCNVAVTQT

SEQ ID NO: 158 - protein, 240 aa, synthetic construct
SYQVGYLHAPTGSGKSTKVPAAYAAQGYKVLVLNPSVAATLGFGAYMSKAHGIDPNIRTGVRTVTTGAPITYSTYGKFLADGGCSGGAYDIIICDECHSVDATTILGIGTVLDQAETAGARLVVLATATPPGSVTVPHPNIEEVALGNEGEIPFYGKAIPLSTIKGGRHLIFCHSKKKCDELAAKLRGMGLNAVAYYRGLDVSVIPTQGDVVVVATDALMTGYTGDFDSVIDCNVAVTQT

SEQ ID NO: 159 - protein, 465 aa, synthetic construct
SFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRI

SEQ ID NO: 160 - protein, 156 aa, Hepatitis C virus
KFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFL

SEQ ID NO: 161 - protein, 228 aa, synthetic construct; identical to SEQ ID NO: 67
  VARIANT 94: note = Wherein X is T or V
  VARIANT 142: note = Wherein X is T, N, Q, H or S
  VARIANT 156: note = Wherein X is E or A
  VARIANT 157: note = Wherein X is V, T, Y, F or L
  VARIANT 202: note = Wherein X is S or Q

SEQ ID NO: 162 - protein, 228 aa, synthetic construct
LHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGFTGDFDSVIDCN

SEQ ID NO: 163 - protein, 25 aa, synthetic construct
  VARIANT 1..25: note = Wherein up to 4 copies of GGGGS may be deleted
GGGGSGGGGSGGGGSGGGGSGGGGS

SEQ ID NO: 164 - protein, 5 aa, synthetic construct
AAAGG

SEQ ID NO: 165 - protein, 25 aa, synthetic construct
  VARIANT 1..25: note = Wherein up to 4 copies of EAAAK may be deleted
EAAAKEAAAKEAAAKEAAAKEAAAK

SEQ ID NO: 166 - protein, 27 aa, synthetic construct
  VARIANT 2..26: note = Wherein up to 4 copies of EAAAK may be deleted
AEAAAKEAAAKEAAAKEAAAKEAAAKA

SEQ ID NO: 167 - protein, 56 aa, synthetic construct
  VARIANT 2..26: note = Wherein up to 4 copies of EAAAK may be deleted
  VARIANT 31..55: note = Wherein up to 4 copies of EAAAK may be deleted
AEAAAKEAAAKEAAAKEAAAKEAAAKALEAEAAAKEAAAKEAAAKEAAAKEAAAKA

SEQ ID NO: 168 - protein, 10 aa, synthetic construct
  VARIANT 3..10: note = Wherein up to 4 copies of KP may be deleted
KPKPKPKPKP

SEQ ID NO: 169 - protein, 10 aa, synthetic construct
  VARIANT 3..10: note = Wherein up to 4 copies of EP may be deleted
EPEPEPEPEP

SEQ ID NO: 170 - protein, 15 aa, synthetic construct
  VARIANT 4..15: note = Wherein up to 4 copies of TPR may be deleted
TPRTPRTPRTPRTPR

SEQ ID NO: 171 - protein, 10 aa, synthetic construct
  VARIANT 3..10: note = Wherein up to 4 copies of AP may be deleted
APAPAPAPAP

SEQ ID NO: 172 - protein, 160 aa, synthetic construct
KSTKVPAAYAAQGYKVLVLNPSVAATLGFGAYMSKLEQIKGGRHLIFCHSKKKCDELAAKLRILRRHVGPGEGAVQWMNRLIAFASRGNHVSPTHYVPESDAAARVTQILGVYLLPRRGPRLGVRATRKTSERSQPRGRRQPIPKARRSEGRSWAQPGYP